(19) United States
(12) Patent Application Publication
(10) Pub. No.: US 2023/0286968 A1
(43) Pub. Date: Sep. 14, 2023

(54) ARYLOXAZOLO SPIRAL RING DERIVATIVES FOR THE TREATMENT AND PROPHYLAXIS OF HEPATITIS B VIRUS INFECTION

(71) Applicant: Hoffmann-La Roche Inc., Little Falls, NJ (US)

(72) Inventors: Xianfeng LIN, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/319,828

(22) Filed: May 18, 2023

Related U.S. Application Data
(63) Continuation of application No. PCT/EP2021/082396, filed on Nov. 22, 2021.

(30) Foreign Application Priority Data
Nov. 24, 2020 (WO) PCT/CN2020/131155

(51) Int. Cl. C07D 413/12; C07D 413/14; C07D 417/14; C07D 263/56; C07D 491/107; C07D 417/12

(52) U.S. Cl. CPC C07D 413/12; C07D 413/14; C07D 417/14; C07D 263/56; C07D 491/107; C07D 417/12

(57) ABSTRACT

The present invention provides novel compounds having the general formula:

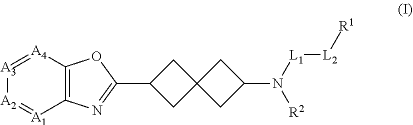

wherein A₁ to A₄, L₁, L₂, R¹ and R² are as described herein, compositions including the compounds and methods of using the compounds.

### CROSS-REFERENCE TO PRIOR APPLICATIONS

This application is a continuation of International Application No. PCT/EP2021/082396 having an International Filing Date of Nov. 22, 2021 and which chaims benefit under 35 U.S.C. § 119 to International Application No. PCT/CN2020/131155 having an International Filing Date of Nov. 24, 2020. The entire contents of both are incorporated herein by reference.

### FIELD OF THE INVENTION

The present invention relates to aryloxazolo spiral ring compounds of formula (I) and their corresponding derivatives useful for therapy and/or prophylaxis of HBV infection in a mammal, and in particular to HBsAg (HBV Surface antigen) and HBeAg (HBV e antigen) inhibitors as well as their manufacture and pharmaceutical compositions containing them.

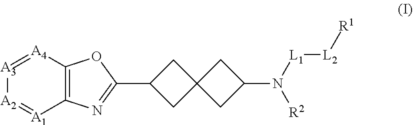

The present invention relates to compounds of formula (I), wherein A₁ to A₄, L₁, L₂, R¹ and R² are as described below, or a pharmaceutically acceptable salt thereof.

### BACKGROUND OF THE INVENTION

Hepatitis B virus (HBV) is one of the most dangerous human pathogens. A safe and effective vaccine has been available for longer than two decades; however, WHO estimated that approximately 257 million people are chronically infected with HBV. Chronic Hepatitis B (CHB) infection predisposes its host to severe liver disease, including liver cirrhosis and hepatocellular carcinoma, if left untreated. HBV infection is ranked among the top unmet medical need worldwide. The currently approved drugs have contributed to substantial progress in CHB treatment; however, the cure rate remains less than 10%.

The control of viral infection needs an effective immune surveillance. Upon recognition of viral infection, the host innate immune system could respond within minutes to impede viral replication and limits the development of a chronic and persistent infection. The secretion of antiviral cytokines from infected hepatocytes and intra-hepatic immune cells is critically important for the clearance of viral infection. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may contribute to immune tolerant state observed in CHB patients. The persistent exposure to HBsAg and other viral antigens can lead to HBV-specific T-cell functional impairment and depletion (Kondo et al. Journal of Immunology (1993), 150, 4659-4671; Kondo et al. Journal of Medical Virology (2004), 74, 425-433; Fisicaro et al. Gastroenterology, (2010), 138, 682-693). Moreover, HBsAg has been reported to suppress immune cell functions, including monocytes, dendritic cells (DCs) and natural killer (NK) cells (Op den Brouw et al. Immunology, (2009b), 126, 280-289; Woltman et al. PLoS One, (2011), 6, e15324; Shi et al. J Viral Hepat. (2012), 19, e26-33; Kondo et al. ISRN Gasteroenterology, (2013), Article ID 935295).

HBsAg is an important biomarker for prognosis and treatment response in CHB. However, the achievement of HBsAg loss and seroconversion is rarely achieved in CHB patients. HBsAg loss with or without anti-HBsAg seroconversion remains the ideal clinical treatment endpoints. Current therapies, such as nucleos(t)ide analogues, are effective in supressing HBV DNA, but are not effective in reducing HBsAg level. Nucleos(t)ide analogs, even with prolonged therapy, have demonstrated HBsAg clearance rates comparable to those observed naturally (Janssen et al. Lancet, (2005), 365, 123-129; Marcellin et al. N. Engl. J. Med., (2004), 351, 1206-1217; Buster et al. Hepatology, (2007), 46, 388-394). Therefore, there is an urgent need for the development of novel therapeutic agents that could efficiently reduce HBsAg. (Wieland, S. F. & F. V. Chisari. J Virol, (2005), 79, 9369-9380; Kumar et al. J Virol, (2011), 85, 987-995; Woltman et al. PLoS One, (2011), 6, e15324; Op den Brouw et al. Immunology, (2009b), 126, 280-289).

### SUMMARY OF THE INVENTION

Objects of the present invention are novel compounds of formula (I), their manufacture, medicaments based on a compound in accordance with the invention and their production as well as the use of compounds of formula (I) as HBV inhibitors and for the treatment or prophylaxis of HBV infection. The compounds of formula (I) show superior anti-HBV activity. In addition, the compounds of formula (I) also show good safety and good PK profiles.

The present invention relates to a compound of formula (I)

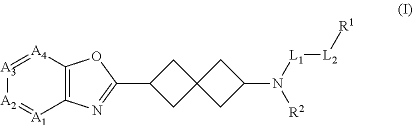

wherein
R¹ is C₁₋₆alkyl, C₃₋₇cycloalkyl, pyridyl, oxopyrrolidinyl, oxopiperidyl, aminosulfonyl, dioxothiolanyl, dioxothiazolidinyl, dioxothietanyl, azetidinyl, dioxo-2lambda6-thiaspiro[3.3]heptanyl, dioxothianyl, dioxothiazinanyl, tetrahydrofuranyl, pyrrolidinyl, 2-oxa-7-azaspiro[3.4]octanyl, piperidyl or C₃₋₇cycloalkylcarbonylaminosulfonyl; wherein C₃₋₇ cycloalkyl, pyridyl, oxopyrrolidinyl, oxopiperidyl, dioxothiazolidinyl, dioxothiolanyl, azetidinyl, dioxothianyl, pyrrolidinyl and piperidyl are unsubstituted or substituted by one or two or three substituents independently selected from OH, C₁₋₆alkyl, haloC₁₋₆alkyl, C₁₋₆alkoxy, oxetanylamino, (C₁₋₆alkyl)₂amino, piperidyl, morpholino, C₃₋₇cycloalkylamino, oxopyrrolidinyl, C₁₋₆alkylthiazolyl, thiazolyl, cyano, aminocarbonyl, aminosulfonyl, C₁₋₆alkylaminosulfonylamino, C₁₋₆alkylsulfonyl, oxetanylsulfonyl, phenyl, halogen(C₁₋₆ alkyl)phenyl, phenylC₁₋₆alkyl, C₃₋₇cycloalkylsulfonyl, C₁₋₆alkylcarbonyl, C₃₋₇ cycloalkylcarbonyl, C₁₋₆alkylphenylsulfonyl, C₃₋₇cycloalkylsulfonimidoyl, C₁₋₆ alkylsulfonimidoyl, dioxothiazinanyl, N-hydroxycarbamimidoyl, carbamimidoyl, C₁₋₆ alkylamino, azetidinylsulfonyl, C₁₋₆alkylcarbonylaminosulfonyl, C₃₋₇cycloalkylC₁₋₆alkylsulfonyl, C₁₋₆alkylaminocarbonyl and (C₁₋₆alkyl)₂aminocarbonyl;
R² is H or C₁₋₆alkyl;
L₁ is —C(O)— or —S(O)₂—;
L₂ is a bond, —(CH₂)ₘ—, —NH—, N(CH₃)— or —NH—CH₂—; wherein m is 1, 2 or 3;
A₁ is N or CR³; wherein R³ is H, halogen, C₁₋₆alkyl or haloC₁₋₆alkyl;
A₂ is N or CR⁴; wherein R⁴ is H, halogen, C₁₋₆alkyl or haloC₁₋₆alkyl;
A₃ is N or CR⁵; wherein R⁵ is H, halogen, C₁₋₆alkyl or haloC₁₋₆alkyl;
A₄ is N or CR⁶; wherein R⁶ is H, halogen, C₁₋₆alkyl or haloC₁₋₆alkyl;
wherein with the proviso that A₁, A₂, A₃ and A₄ are not CH simultaneously;
or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 2 to 6 or 1 to 4 carbon atoms, for example methyl, ethyl, propyl, isopropyl, butyl, isobutyl, tert-butyl and the like. Particular “C₁₋₆ alkyl” groups are methyl and ethyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy and propoxy.

The term “halogen” denotes fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl.

The term “C₃₋₇cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” group is cyclopropyl.

The term “carbonyl” alone or in combination refers to the group —C(O)—.

The term “sulfonyl” alone or in combination refers to the group —S(O)₂—.

The term “sulfonimidoyl” alone or in combination refers to the group —S(O)(NH)—, whose formula is

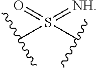 [SMILES: *S(*)(=N)=O]

The term “bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

### HBV Inhibitors

The present invention provides (i) a compound having the general formula (I):

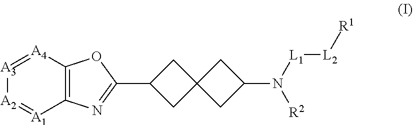

wherein
R¹ is C₁₋₆alkyl, C₃₋₇cycloalkyl, pyridyl, oxopyrrolidinyl, oxopiperidyl, aminosulfonyl, dioxothiolanyl, dioxothiazolidinyl, dioxothietanyl, azetidinyl, dioxo-2lambda6-thiaspiro[3.3]heptanyl, dioxothianyl, dioxothiazinanyl, tetrahydrofuranyl, pyrrolidinyl, 2-oxa-7-azaspiro[3.4]octanyl, piperidyl or C₃₋₇cycloalkylcarbonylaminosulfonyl; wherein C₃₋₇cycloalkyl, pyridyl, oxopyrrolidinyl, oxopiperidyl, dioxothiazolidinyl, dioxothiolanyl, azetidinyl, dioxothianyl, pyrrolidinyl and piperidyl are unsubstituted or substituted by one or two or three substituents independently selected from OH, C₁₋₆alkyl, haloC₁₋₆alkyl, C₁₋₆alkoxy, oxetanylamino, (C₁₋₆alkyl)₂amino, piperidyl, morpholino, C₃₋₇cycloalkylamino, oxopyrrolidinyl, C₁₋₆alkylthiazolyl, thiazolyl, cyano, aminocarbonyl, aminosulfonyl, C₁₋₆ alkylaminosulfonylamino, C₁₋₆alkylsulfonyl, oxetanylsulfonyl, phenyl, halogen(C₁₋₆alkyl)phenyl, phenylC₁₋₆alkyl, C₃₋₇cycloalkylsulfonyl, C₁₋₆alkylcarbonyl, C₃₋₇ cycloalkylcarbonyl, C₁₋₆alkylphenylsulfonyl, C₃₋₇cycloalkylsulfonimidoyl, C₁₋₆ alkylsulfonimidoyl, dioxothiazinanyl, N-hydroxycarbamimidoyl, carbamimidoyl, C₁₋₆ alkylamino, azetidinylsulfonyl, C₁₋₆alkylcarbonylaminosulfonyl, C₃₋₇cycloalkylC₁₋₆alkylsulfonyl, C₁₋₆alkylaminocarbonyl and (C₁₋₆alkyl)₂aminocarbonyl;
R² is H or C₁₋₆alkyl;
L₁ is —C(O)— or —S(O)₂—;
L₂ is a bond, —(CH₂)ₘ—, —NH—, N(CH₃)— or —NH—CH₂—; wherein m is 1, 2 or 3;
A₁ is N or CR³; wherein R³ is H, halogen, C₁₋₆alkyl or haloC₁₋₆alkyl;
A₂ is N or CR⁴; wherein R⁴ is H, halogen, C₁₋₆alkyl or haloC₁₋₆alkyl;
A₃ is N or CR⁵; wherein R⁵ is H, halogen, C₁₋₆alkyl or haloC₁₋₆alkyl;
A₄ is N or CR⁶; wherein R⁶ is H, halogen, C₁₋₆alkyl or haloC₁₋₆alkyl;
wherein with the proviso that A₁, A₂, A₃ and A₄ are not CH simultaneously;
or a pharmaceutically acceptable salt thereof.

A further embodiment of the present invention is (ii) a compound of formula (I) according to (i), wherein A₁ is N or CH.

A further embodiment of the present invention is (iii) a compound of formula (I) according to any one of (i)-(ii), wherein A₁ is CH.

A further embodiment of the present invention is (iv) a compound of formula (I) according to any one of (i)-(iii), or a pharmaceutically acceptable salt thereof, wherein A₂ is CR⁴; wherein R⁴ is H, halogen or haloC₁₋₆alkyl.

A further embodiment of the present invention is (v) a compound of formula (I) according to any one of (i)-(iv), or a pharmaceutically acceptable salt thereof, wherein A₂ is CR⁴; wherein R⁴ is halogen.

A further embodiment of the present invention is (vi) a compound of formula (I) according to any one of (i)-(v), or a pharmaceutically acceptable salt thereof, wherein A₂ is CCl.

A further embodiment of the present invention is (vii) a compound of formula (I) according to any one of (i)-(vi), or a pharmaceutically acceptable salt thereof, wherein A₃ is CH.

A further embodiment of the present invention is (viii) a compound of formula (I) according to any one of (i)-(vii), or a pharmaceutically acceptable salt thereof, wherein A₄ is N or CH.

A further embodiment of the present invention is (ix) a compound of formula (I) according to any one of (i)-(viii), or a pharmaceutically acceptable salt thereof, wherein R² is H or methyl.

A further embodiment of the present invention is (x) a compound of formula (I) according to any one of (i)-(ix), or a pharmaceutically acceptable salt thereof, wherein R² is H.

A further embodiment of the present invention is (xi) a compound of formula (I) according to any one of (i)-(x), or a pharmaceutically acceptable salt thereof, wherein L₁ is —C(O)—.

A further embodiment of the present invention is (xii) a compound of formula (I) according to any one of (i)-(xi), or a pharmaceutically acceptable salt thereof, wherein L₂ is a bond.

A further embodiment of the present invention is (xiii) a compound of formula (I) according to any one of (i)-(xii), or a pharmaceutically acceptable salt thereof, wherein R¹ is pyridyl; wherein pyridyl is substituted by one substituent selected from aminocarbonyl, C₁₋₆alkylsulfonyl, C₃₋₇cycloalkylsulfonyl, C₃₋₇cycloalkylsulfonimidoyl and C₃₋₇cycloalkylC₁₋₆alkylsulfonyl.

A further embodiment of the present invention is (xiv) a compound of formula (I) according to any one of (i)-(xiii), or a pharmaceutically acceptable salt thereof, wherein R¹ is pyridyl; wherein pyridyl is substituted by one substituent selected from aminocarbonyl, methylsulfonyl, ethylsulfonyl, cyclopropylsulfonyl, cyclopropylsulfonimidoyl and cyclopropylmethylsulfonyl.

A further embodiment of the present invention is (xv) a compound of formula (I) according to (i), or a pharmaceutically acceptable salt thereof, wherein
R¹ is C₁₋₆alkyl, C₃₋₇cycloalkyl, pyridyl, oxopyrrolidinyl, oxopiperidyl, aminosulfonyl, dioxothiolanyl, dioxothiazolidinyl, dioxothietanyl, azetidinyl, dioxo-2lambda6-thiaspiro[3.3]heptanyl, dioxothianyl, dioxothiazinanyl, tetrahydrofuranyl, pyrrolidinyl, 2-oxa-7-azaspiro[3.4]octanyl, piperidyl or C₃₋₇cycloalkylcarbonylaminosulfonyl; wherein C₃₋₇cycloalkyl, pyridyl, oxopyrrolidinyl, oxopiperidyl, dioxothiazolidinyl, dioxothiolanyl, azetidinyl, dioxothianyl, pyrrolidinyl and piperidyl are unsubstituted or substituted by one or two or three substituents independently selected from OH, C₁₋₆alkyl, haloC₁₋₆alkyl, C₁₋₆alkoxy, oxetanylamino, (C₁₋₆alkyl)₂amino, piperidyl, morpholino, C₃₋₇cycloalkylamino, oxopyrrolidinyl, C₁₋₆alkylthiazolyl, thiazolyl, cyano, aminocarbonyl, aminosulfonyl, C₁₋₆ alkylaminosulfonylamino, C₁₋₆alkylsulfonyl, oxetanylsulfonyl, phenyl, halogen(C₁₋₆alkyl)phenyl, phenylC₁₋₆alkyl, C₃₋₇cycloalkylsulfonyl, C₁₋₆alkylcarbonyl, C₃₋₇ cycloalkylcarbonyl, C₁₋₆alkylphenylsulfonyl, C₃₋₇cycloalkylsulfonimidoyl, C₁₋₆alkylsulfonimidoyl, dioxothiazinanyl, N-hydroxycarbamimidoyl, carbamimidoyl, C₁₋₆alkylamino, azetidinylsulfonyl, C₁₋₆alkylcarbonylaminosulfonyl, C₃₋₇cycloalkylC₁₋₆alkylsulfonyl, C₁₋₆alkylaminocarbonyl and (C₁₋₆alkyl)₂aminocarbonyl;
R² is H or C₁₋₆alkyl;
L₁ is —C(O)— or —S(O)₂—;
L₂ is a bond, —(CH₂)ₘ—, —NH—, N(CH₃)— or —NH—CH₂—; wherein m is 1, 2 or 3;
A₁ is N or CH;
A₂ is CR⁴; wherein R⁴ is H, halogen or haloC₁₋₆alkyl;
A₃ is CH;
A₄ is N or CH;
wherein with the proviso that A₁, A₂, A₃ and A₄ are not CH simultaneously.

A further embodiment of the present invention is (xvi) a compound of formula (I) according to (i), or a pharmaceutically acceptable salt thereof,
R¹ pyridyl; wherein pyridyl is substituted by one substituent selected from aminocarbonyl, C₁₋₆alkylsulfonyl, C₃₋₇cycloalkylsulfonyl, C₃₋₇cycloalkylsulfonimidoyl and C₃₋₇cycloalkylC₁₋₆alkylsulfonyl;
R² is H;
L₁ is —C(O)—;
L₂ is a bond;
A₁ is CH;
A₂ is CR⁴; wherein R⁴ is halogen;
A₃ is CH;
A₄ is N or CH;
wherein with the proviso that A₁, A₂, A₃ and A₄ are not CH simultaneously.

A further embodiment of the present invention is (xvii) a compound of formula (I) according to (i), or a pharmaceutically acceptable salt thereof, wherein
R¹ is pyridyl; wherein pyridyl is substituted by one substituent selected from aminocarbonyl, methylsulfonyl, ethylsulfonyl, cyclopropylsulfonyl, cyclopropylsulfonimidoyl and cyclopropylmethylsulfonyl;
R² is H;
L₁ is —C(O)—;
L₂ is a bond;
A₁ is CH;
A₂ is CCl;
A₃ is CH;
A₄ is N or CH;
wherein with the proviso that A₁, A₂, A₃ and A₄ are not CH simultaneously.

In another embodiment (xiii) of the present invention, particular compounds of the present invention are selected from:
N-[6-(5-chloro-1,3-benzoxazol-2-yl)spiro[3.3]heptan-2-yl]pyridine-2-carboxamide;
N-[6-(5-chloro-1,3-benzoxazol-2-yl)spiro[3.3]heptan-2-yl]pyridine-3-carboxamide;
N-[6-(5-chloro-1,3-benzoxazol-2-yl)spiro[3.3]heptan-2-yl]pyridine-4-carboxamide;
N-[6-(5-chloro-1,3-benzoxazol-2-yl)spiro[3.3]heptan-2-yl]-2-methyl-pyridine-4-carboxamide;
N-[6-(5-chloro-1,3-benzoxazol-2-yl)spiro[3.3]heptan-2-yl]-2-(oxetan-3-ylamino)pyridine-4-carboxamide;
N-[6-(5-chloro-1,3-benzoxazol-2-yl)spiro[3.3]heptan-2-yl]-2-(diethylamino)pyridine-4-carboxamide;
N-[6-(5-chloro-1,3-benzoxazol-2-yl)spiro[3.3]heptan-2-yl]-2-[ethyl(methyl)amino]pyridine-4-carboxamide;
N-[6-(5-chloro-1,3-benzoxazol-2-yl)spiro[3.3]heptan-2-yl]-2-(1-piperidyl)pyridine-4-carboxamide;
N-[6-(5-chloro-1,3-benzoxazol-2-yl)spiro[3.3]heptan-2-yl]-2-morpholino-pyridine-4-carboxamide;
N-[6-(5-chloro-1,3-benzoxazol-2-yl)spiro[3.3]heptan-2-yl]-2-(cyclopropylamino)pyridine-4-carboxamide;
N-[6-(5-chloro-1,3-benzoxazol-2-yl)spiro[3.3]heptan-2-yl]-2-(dimethylamino)pyridine-4-carboxamide;
N-[6-(5-chloro-1,3-benzoxazol-2-yl)spiro[3.3]heptan-2-yl]-2-[isopropyl(methyl)amino]pyridine-4-carboxamide;
N-[6-(5-chloro-1,3-benzoxazol-2-yl)spiro[3.3]heptan-2-yl]-2-(2-oxopyrrolidin-1-yl)pyridine-4-carboxamide;
N-[6-(5-chloro-1,3-benzoxazol-2-yl)spiro[3.3]heptan-2-yl]-2-(4-methylthiazol-2-yl)pyridine-4-carboxamide;
N-[6-(5-chloro-1,3-benzoxazol-2-yl)spiro[3.3]heptan-2-yl]-2-(5-methylthiazol-2-yl)pyridine-4-carboxamide;
N-[6-(5-chloro-1,3-benzoxazol-2-yl)spiro[3.3]heptan-2-yl]-2-thiazol-2-yl-pyridine-4-carboxamide;
N-[6-(5-chloro-1,3-benzoxazol-2-yl)spiro[3.3]heptan-2-yl]-2-methoxy-pyridine-4-carboxamide;
N-[6-(5-chloro-1,3-benzoxazol-2-yl)spiro[3.3]heptan-2-yl]-2-cyano-pyridine-4-carboxamide;
N-[6-(5-chloro-1,3-benzoxazol-2-yl)spiro[3.3]heptan-2-yl]-2-sulfamoyl-pyridine-4-carboxamide;
N-[6-(5-chloro-1,3-benzoxazol-2-yl)spiro[3.3]heptan-2-yl]-2-isopropoxy-pyridine-4-carboxamide;
N-[6-(5-chloro-1,3-benzoxazol-2-yl)spiro[3.3]heptan-2-yl]-2-(trifluoromethyl)pyridine-4-carboxamide;
N-[6-(5-chloro-1,3-benzoxazol-2-yl)spiro[3.3]heptan-2-yl]-2-(methylsulfamoylamino)pyridine-4-carboxamide;
N-[6-(5-chloro-1,3-benzoxazol-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-5-methylsulfonyl-pyridine-3-carboxamide;
N-[6-(5-chloro-1,3-benzoxazol-2-yl)spiro[3.3]heptan-2-yl]-2-(oxetan-3-ylsulfonyl)pyridine-4-carboxamide;
N-[6-(5-chloro-1,3-benzoxazol-2-yl)spiro[3.3]heptan-2-yl]-2-ethylsulfonyl-pyridine-4-carboxamide;
N-[6-(5-chloro-1,3-benzoxazol-2-yl)spiro[3.3]heptan-2-yl]-2,2-dimethyl-propanamide;
N-[6-(5-chloro-1,3-benzoxazol-2-yl)spiro[3.3]heptan-2-yl]-3-methyl-butanamide;
N-[6-(5-chloro-1,3-benzoxazol-2-yl)spiro[3.3]heptan-2-yl]-3-(trifluoromethyl)cyclobutanecarboxamide;
N-[6-(5-chloro-1,3-benzoxazol-2-yl)spiro[3.3]heptan-2-yl]-1-methyl-5-oxo-pyrrolidine-3-carboxamide;
1-tert-butyl-N-[6-(5-chloro-1,3-benzoxazol-2-yl)spiro[3.3]heptan-2-yl]-5-oxo-pyrrolidine-3-carboxamide;
N-[6-(5-chloro-1,3-benzoxazol-2-yl)spiro[3.3]heptan-2-yl]-5-oxo-1-phenyl-pyrrolidine-3-carboxamide;
N-[6-(5-chloro-1,3-benzoxazol-2-yl)spiro[3.3]heptan-2-yl]-1-(3-chloro-2-methyl-phenyl)-5-oxo-pyrrolidine-3-carboxamide;
N-[6-(5-chloro-1,3-benzoxazol-2-yl)spiro[3.3]heptan-2-yl]-1-methyl-2-oxo-piperidine-4-carboxamide;
N-[6-(5-chloro-1,3-benzoxazol-2-yl)spiro[3.3]heptan-2-yl]-2-(1-methyl-5-oxo-pyrrolidin-3-yl)acetamide;
N-[6-(5-chloro-1,3-benzoxazol-2-yl)spiro[3.3]heptan-2-yl]-2-(1-methyl-5-oxo-pyrrolidin-2-yl)acetamide;
N-[6-(5-chloro-1,3-benzoxazol-2-yl)spiro[3.3]heptan-2-yl]-2-(5-oxopyrrolidin-2-yl)acetamide;
N-[6-(5-chloro-1,3-benzoxazol-2-yl)spiro[3.3]heptan-2-yl]-3-sulfamoyl-propanamide;
N-[6-(5-chloro-1,3-benzoxazol-2-yl)spiro[3.3]heptan-2-yl]-4-sulfamoyl-butanamide;
N-[6-(5-chloro-1,3-benzoxazol-2-yl)spiro[3.3]heptan-2-yl]-1,1-dioxo-thiolane-3-carboxamide;
N-(6-oxazolo[4,5-b]pyridin-2-ylspiro[3.3]heptan-2-yl)-1,1-dioxo-thiolane-3-carboxamide;
N-[6-(5-chloro-1,3-benzoxazol-2-yl)spiro[3.3]heptan-2-yl]-1,1-dioxo-thiolane-2-carboxamide;
N-[6-(5-chloro-1,3-benzoxazol-2-yl)spiro[3.3]heptan-2-yl]-1,1-dioxo-1,2-thiazolidine-4-carboxamide;
2-benzyl-N-[6-(5-chloro-1,3-benzoxazol-2-yl)spiro[3.3]heptan-2-yl]-1,1-dioxo-1,2-thiazolidine-4-carboxamide;
N-[6-(5-chloro-1,3-benzoxazol-2-yl)spiro[3.3]heptan-2-yl]-1,1-dioxo-1,2-thiazolidine-3-carboxamide;
N-[6-(5-chloro-1,3-benzoxazol-2-yl)spiro[3.3]heptan-2-yl]-2-methyl-1,1-dioxo-1,2-thiazolidine-3-carboxamide;
N-[6-(5-chloro-1,3-benzoxazol-2-yl)spiro[3.3]heptan-2-yl]-2-(1,1-dioxothiolan-2-yl)acetamide;
N-(6-(5-chlorobenzo[d]oxazol-2-yl)spiro[3.3]heptan-2-yl)-3-methyltetrahydrothiophene-3-carboxamide 1,1-dioxide;
N-[6-(5-chloro-1,3-benzoxazol-2-yl)spiro[3.3]heptan-2-yl]-1,1-dioxo-thietane-3-carboxamide;
N-[6-(5-chloro-1,3-benzoxazol-2-yl)spiro[3.3]heptan-2-yl]-2-(1,1-dioxothietan-3-yl)acetamide;
N-[6-(5-chloro-1,3-benzoxazol-2-yl)spiro[3.3]heptan-2-yl]oxetane-3-carboxamide;
N-[6-(5-chloro-1,3-benzoxazol-2-yl)spiro[3.3]heptan-2-yl]-2,2-dioxo-2-thiaspiro[3.3]heptane-6-carboxamide;
N-[6-(5-chloro-1,3-benzoxazol-2-yl)spiro[3.3]heptan-2-yl]-1,1-dioxo-thiane-4-carboxamide;
N-(6-(5-chlorobenzo[d]oxazol-2-yl)spiro[3.3]heptan-2-yl)-4-methyltetrahydro-2H-thiopyran-4-carboxamide 1,1-dioxide;
N-[6-(5-chloro-1,3-benzoxazol-2-yl)spiro[3.3]heptan-2-yl]-2-(1,1-dioxothian-3-yl)acetamide;
N-[6-(5-chloro-1,3-benzoxazol-2-yl)spiro[3.3]heptan-2-yl]-2-(1,1-dioxothiazinan-2-yl)acetamide;
1,1-dioxo-N-[6-[5-(trifluoromethyl)-1,3-benzoxazol-2-yl]spiro[3.3]heptan-2-yl]thiane-4-carboxamide;
N-[6-(5-chloro-1,3-benzoxazol-2-yl)spiro[3.3]heptan-2-yl]propane-2-sulfonamide;
1-[6-(5-chloro-1,3-benzoxazol-2-yl)spiro[3.3]heptan-2-yl]-3-ethyl-urea;
1-[6-(5-chloro-1,3-benzoxazol-2-yl)spiro[3.3]heptan-2-yl]-3-propyl-urea;
1-[6-(5-chloro-1,3-benzoxazol-2-yl)spiro[3.3]heptan-2-yl]-3-isopropyl-urea;
3-[6-(5-chloro-1,3-benzoxazol-2-yl)spiro[3.3]heptan-2-yl]-1-ethyl-1-methyl-urea;
1-[6-(5-chloro-1,3-benzoxazol-2-yl)spiro[3.3]heptan-2-yl]-3-(tetrahydrofuran-3-ylmethyl)urea;
1-[6-(5-chloro-1,3-benzoxazol-2-yl)spiro[3.3]heptan-2-yl]-3-[(3-hydroxycyclobutyl)methyl]urea;
1-[6-(5-chloro-1,3-benzoxazol-2-yl)spiro[3.3]heptan-2-yl]-3-tetrahydrofuran-3-yl-urea;
N-[6-(5-chloro-1,3-benzoxazol-2-yl)spiro[3.3]heptan-2-yl]pyrrolidine-1-carboxamide;
N-[6-(5-chloro-1,3-benzoxazol-2-yl)spiro[3.3]heptan-2-yl]-2-oxa-7-azaspiro[3.4]octane-7-carboxamide;
N-[6-(5-chloro-1,3-benzoxazol-2-yl)spiro[3.3]heptan-2-yl]-1,1-dioxo-1,4-thiazinane-4-carboxamide;
N-[6-(5-chloro-1,3-benzoxazol-2-yl)spiro[3.3]heptan-2-yl]-1-methylsulfonyl-pyrrolidine-3-carboxamide;
N-[6-(5-chloro-1,3-benzoxazol-2-yl)spiro[3.3]heptan-2-yl]-1-ethylsulfonyl-pyrrolidine-3-carboxamide;
N-[6-(5-chloro-1,3-benzoxazol-2-yl)spiro[3.3]heptan-2-yl]-1-propylsulfonyl-pyrrolidine-3-carboxamide;
N-[6-(5-chloro-1,3-benzoxazol-2-yl)spiro[3.3]heptan-2-yl]-1-cyclopropylsulfonyl-pyrrolidine-3-carboxamide;
1-acetyl-N-[6-(5-chloro-1,3-benzoxazol-2-yl)spiro[3.3]heptan-2-yl]pyrrolidine-3-carboxamide;
N-[6-(5-chloro-1,3-benzoxazol-2-yl)spiro[3.3]heptan-2-yl]-1-(cyclopropanecarbonyl)pyrrolidine-3-carboxamide;
N-[6-(5-chloro-1,3-benzoxazol-2-yl)spiro[3.3]heptan-2-yl]-1-(cyclobutanecarbonyl)pyrrolidine-3-carboxamide;
N-[6-(5-chloro-1,3-benzoxazol-2-yl)spiro[3.3]heptan-2-yl]-2-[1-(p-tolylsulfonyl)pyrrolidin-3-yl]acetamide;
N-[6-(5-chloro-1,3-benzoxazol-2-yl)spiro[3.3]heptan-2-yl]-2-(1-methylsulfonylazetidin-3-yl)acetamide;
N-[6-(5-chloro-1,3-benzoxazol-2-yl)spiro[3.3]heptan-2-yl]-2-[1l-(p-tolylsulfonyl)azetidin-3-yl]acetamide;
N-[6-(5-chloro-1,3-benzoxazol-2-yl)spiro[3.3]heptan-2-yl]-2-(1-methylsulfonyl-4-piperidyl)acetamide;
N-[6-(5-chloro-1,3-benzoxazol-2-yl)spiro[3.3]heptan-2-yl]-2-[1-(p-tolylsulfonyl)-4-piperidyl]acetamide;
N-[4-[[6-(5-chloro-1,3-benzoxazol-2-yl)spiro[3.3]heptan-2-yl]amino]-4-oxo-butyl]sulfonylcyclobutanecarboxamide;
(Rₐ)—N-[6-(5-chloro-1,3-benzoxazol-2-yl)spiro[3.3]heptan-2-yl]-1,1-dioxo-thiane-4-carboxamide;
(Sₐ)—N-[6-(5-chloro-1,3-benzoxazol-2-yl)spiro[3.3]heptan-2-yl]-1,1-dioxo-thiane-4-carboxamide;
N-[6-(5-chloro-1,3-benzoxazol-2-yl)spiro[3.3]heptan-2-yl]-2-cyclopropylsulfonyl-pyridine-4-carboxamide;
(Rₐ)—N-[6-(5-chloro-1,3-benzoxazol-2-yl)spiro[3.3]heptan-2-yl]-2-cyclopropylsulfonyl-pyridine-4-carboxamide;
(Sₐ)—N-[6-(5-chloro-1,3-benzoxazol-2-yl)spiro[3.3]heptan-2-yl]-2-cyclopropylsulfonyl-pyridine-4-carboxamide;
N-[6-(6-chlorooxazolo[5,4-b]pyridin-2-yl)spiro[3.3]heptan-2-yl]-2-cyclopropylsulfonyl-pyridine-4-carboxamide;
N-[6-(5-chloro-1,3-benzoxazol-2-yl)spiro[3.3]heptan-2-yl]-2-(cyclopropylsulfonimidoyl)pyridine-4-carboxamide;
(Rₐ)—N-[6-(5-chloro-1,3-benzoxazol-2-yl)spiro[3.3]heptan-2-yl]-2-methylsulfonyl-pyridine-4-carboxamide;
(Sₐ)—N-[6-(5-chloro-1,3-benzoxazol-2-yl)spiro[3.3]heptan-2-yl]-2-methylsulfonyl-pyridine-4-carboxamide;
N-[6-(6-chlorooxazolo[5,4-b]pyridin-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-2-(methylsulfonimidoyl)pyridine-4-carboxamide;
5-chloro-N-[6-(5-chloro-1,3-benzoxazol-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-2-(1,1-dioxo-1,4-thiazinan-4-yl)pyridine-4-carboxamide;
N-[6-(5-chloro-1,3-benzoxazol-2-yl)spiro[3.3]heptan-2-yl]-N-methyl-2-methylsulfonyl-pyridine-4-carboxamide;
N-[6-(5-chloro-1,3-benzoxazol-2-yl)spiro[3.3]heptan-2-yl]-2-ethylsulfonyl-N-methyl-pyridine-4-carboxamide;
(Rₐ)-(3R)—N-[6-(5-chloro-1,3-benzoxazol-2-yl)spiro[3.3]heptan-2-yl]-1-methyl-5-oxo-pyrrolidine-3-carboxamide;
(Rₐ)-(3S)—N-[6-(5-chloro-1,3-benzoxazol-2-yl)spiro[3.3]heptan-2-yl]-1-methyl-5-oxo-pyrrolidine-3-carboxamide;
(Sₐ)-(3R)—N-[6-(5-chloro-1,3-benzoxazol-2-yl)spiro[3.3]heptan-2-yl]-1-methyl-5-oxo-pyrrolidine-3-carboxamide;
(Sₐ)-(3S)—N-[6-(5-chloro-1,3-benzoxazol-2-yl)spiro[3.3]heptan-2-yl]-1-methyl-5-oxo-pyrrolidine-3-carboxamide;
N-[6-(5-chloro-1,3-benzoxazol-2-yl)spiro[3.3]heptan-2-yl]-2-(N-hydroxycarbamimidoyl)pyridine-4-carboxamide;
2-carbamimidoyl-N-[6-(5-chloro-1,3-benzoxazol-2-yl)spiro[3.3]heptan-2-yl]pyridine-4-carboxamide;
N-[6-(5-chloro-1,3-benzoxazol-2-yl)spiro[3.3]heptan-2-yl]-2-(methylamino)pyridine-4-carboxamide;
2-(azetidin-3-ylsulfonyl)-N-[6-(5-chloro-1,3-benzoxazol-2-yl)spiro[3.3]heptan-2-yl]pyridine-4-carboxamide;
2-(acetylsulfamoyl)-N-[6-(5-chloro-1,3-benzoxazol-2-yl)spiro[3.3]heptan-2-yl]pyridine-4-carboxamide;
N-[6-(5-chloro-1,3-benzoxazol-2-yl)spiro[3.3]heptan-2-yl]-2-(cyclopropylmethylsulfonyl)pyridine-4-carboxamide;
N4-[6-(5-chloro-1,3-benzoxazol-2-yl)spiro[3.3]heptan-2-yl]pyridine-2,4-dicarboxamide;
N4-[6-(5-chloro-1,3-benzoxazol-2-yl)spiro[3.3]heptan-2-yl]-N2-methyl-pyridine-2,4-dicarboxamide; and
N4-[6-(5-chloro-1,3-benzoxazol-2-yl)spiro[3.3]heptan-2-yl]-N2,N2-dimethyl-pyridine-2,4-dicarboxamide;
   or a pharmaceutically acceptable salt thereof.

In another embodiment (xix) of the present invention, particular compounds of the present invention are selected from:
N-[6-(5-chloro-1,3-benzoxazol-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-2-ethylsulfonyl-pyridine-4-carboxamide;
N-[6-(5-chloro-1,3-benzoxazol-2-yl)spiro[3.3]heptan-2-yl]-2-cyclopropylsulfonyl-pyridine-4-carboxamide
(Rₐ)—N-[6-(5-chloro-1,3-benzoxazol-2-yl)spiro[3.3]heptan-2-yl]-2-cyclopropylsulfonyl-pyridine-4-carboxamide;
(Sₐ)—N-[6-(5-chloro-1,3-benzoxazol-2-yl)spiro[3.3]heptan-2-yl]-2-cyclopropylsulfonyl-pyridine-4-carboxamide;
N-[6-(6-chlorooxazolo[5,4-b]pyridin-2-yl)spiro[3.3]heptan-2-yl]-2-cyclopropylsulfonyl-pyridine-4-carboxamide;
N-[6-(5-chloro-1,3-benzoxazol-2-yl)spiro[3.3]heptan-2-yl]-2-(cyclopropylsulfonimidoyl)pyridine-4-carboxamide;
(Rₐ)—N-[6-(5-chloro-1,3-benzoxazol-2-yl)spiro[3.3]heptan-2-yl]-2-methylsulfonyl-pyridine-4-carboxamide;
(Sₐ)—N-[6-(5-chloro-1,3-benzoxazol-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-2-(cyclopropylmethylsulfonyl)pyridine-4-carboxamide; and
N4-[6-(5-chloro-1,3-benzoxazol-2-yl)spiro[3.3]heptan-2-yl]pyridine-2,4-dicarboxamide;
   or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, A₁ to A₄, L₁, L₂, R¹ and R² are as defined above unless otherwise indicated. Furthermore, and unless explicitly otherwise stated, all reactions, reaction conditions, abbreviations and symbols have the meanings well known to a person of ordinary skill in organic chemistry.

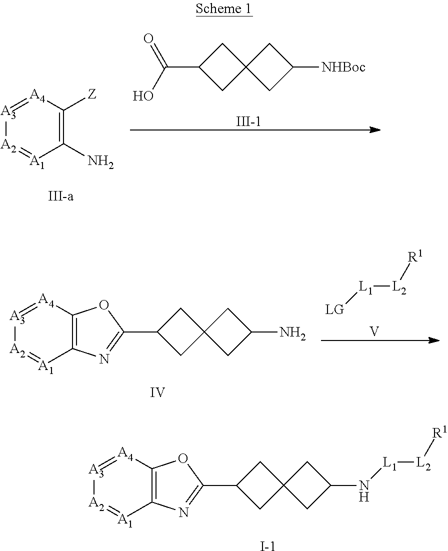

Wherein Z is halogen or OH; LG is Cl or OH.

A compound of formula III-a is heated with a carboxylic acid III-1 in the presence of an acid, such as polyphosphoric acid, to give a compound of formula IV, which then reacts with a compound of formula V in the presence of a coupling reagent, such as HATU or T₃P, and a base, such as TEA or DIPEA, in a solvent, such as DMF or DCM, to afford a compound of formula I.

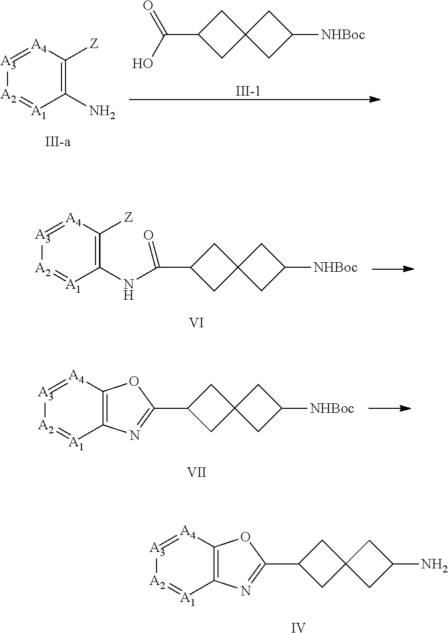

Wherein Z is halogen or OH; LG is Cl or OH.

A compound of formula III-a reacts with a carboxylic acid III-1 in the presence of a coupling reagent such as HATU or T₃P, and a base such as TEA or DIPEA, in a solvent such as DMF or DCM, to afford a compound of formula VI, which then affords a compound of formula VII in presence of DIAD and PPh₃, in a suitable solvent such as THF. The compound of formula IV is formed in presence of an acid such as TFA or HCl.

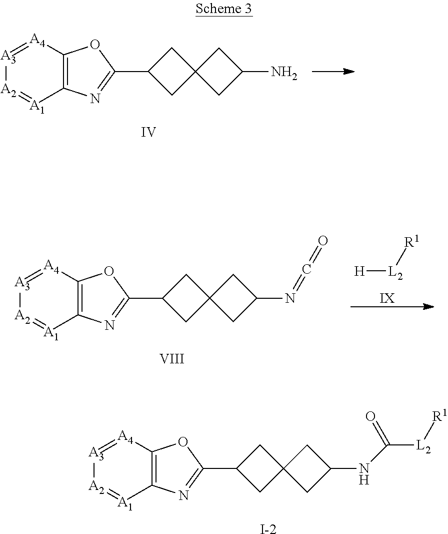

A compound of formula IV reacts with triphosgene in the presence of a base such as NaHCO₃, in a solvent such as DCM, to afford a compound of formula VIII, which then reacts with a compound of formula IX in a suitable solvent such as DCM to afford a compound of formula I-2.

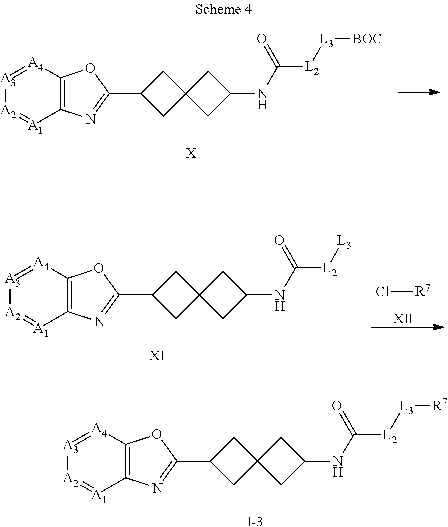

Wherein L₃ is azetidinyl, pyrrolidinyl, piperidyl or —S(O)₂NH₂; R⁷ is C₃₋₇cycloalkylsulfonyl, C₁₋₆alkylcarbonyl, C₃₋₇cycloalkylcarbonyl or C₁₋₆alkylphenylsulfonyl.

A compound of formula X is treated with an acid such as TFA, to afford a compound of formula XI, which then reacts with a compound of formula XII in the presence of a base such as TEA or DIPEA, in a solvent such as DMF or DCM, to afford a compound of formula I-3.

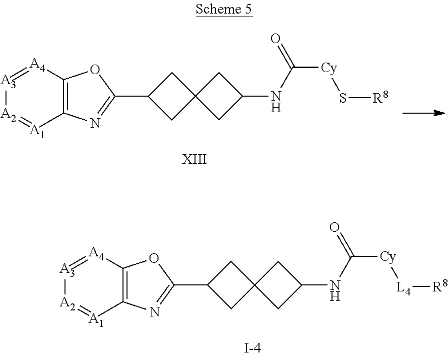

Wherein Cy is pyridyl or chloropyridyl; L₄ is S(O)₂ or S(O)(NH); R⁸ is C₁₋₆alkyl, C₃₋₇cycloalkyl.

Oxidation of a compound of formula XIII in the presence of an oxidate, such as m-CPBA or PhI(OAc)₂, and (NH₄)₂CO₃, affords a compound of formula I-4.

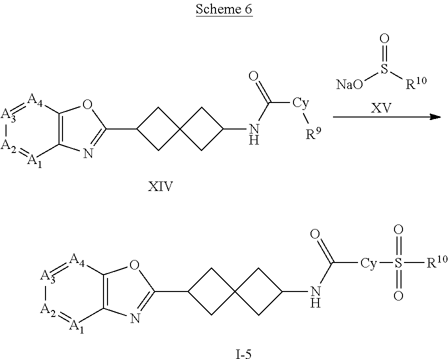

Wherein Cy is pyridyl or chloropyridyl; R⁹ is halogen; R¹⁰ is C₁₋₆alkyl, C₃₋₇cycloalkyl.

A compound of formula XIV reacts with a salt XV in the presence of a catalysts, such as cuprous iodide or L-Proline, and a base such as K₂CO₃, in a solvent such as DMSO, to afford a compound of formula I-5.

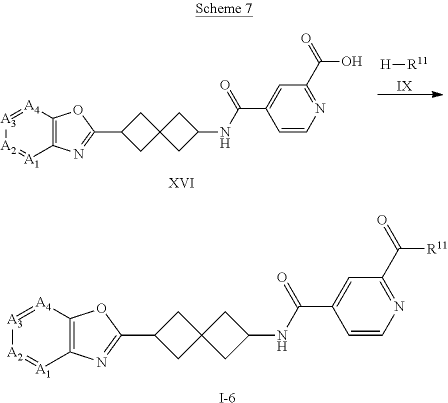

Wherein R¹¹ is —NH, —NHCH₃ or —N(CH₃)₂.

A compound of formula XVI reacts with an amine IX in the presence of a coupling reagent such as HATU or T₃P, and a base such as TEA or DIPEA, in a solvent such as DMF or DCM, to afford a compound of formula I-6.

This invention also relates to a process for the preparation of a compound of formula (I) comprising the following step:
(a) Reaction of a compound of formula (IV),

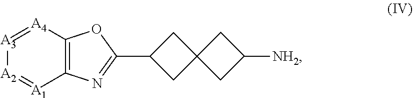

with a compound of formula (V),

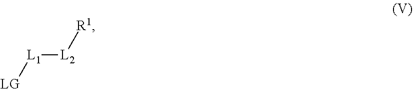

in the presence of a base;
(b) Reaction of a compound of formula (VIII),

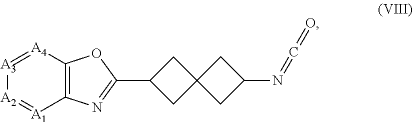

with a compound of formula (IX),

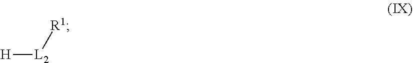

(c) Reaction of a compound of formula (XI),

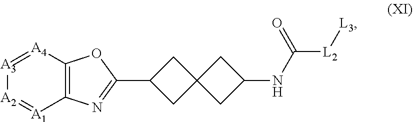

with a compound of formula (XII), Cl—R⁷ (XII); in the presence of a base;
(d) Oxidation of a compound of formula (XIII),

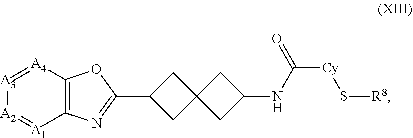

in the presence of an oxidate and (NH₄)₂CO;
(e) Reaction of a compound of formula (XIV),

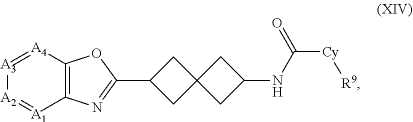

with a compound of formula (XV),

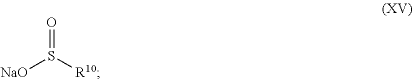

in the presence of an catalysts and a base;
(f) Reaction of a compound of formula (XVI),

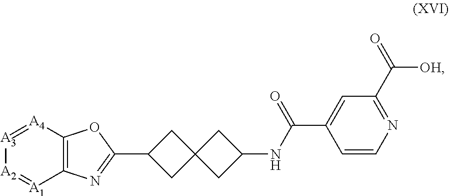

with a compound of formula (IX), H—R¹¹ (IX); in the presence of a coupling reagent and a base;
wherein L₃ is azetidinyl, pyrrolidinyl, piperidyl or —S(O)₂NH₂; L₄ is S(O)₂ or S(O)(NH); R⁷ is C₃₋₇cycloalkylsulfonyl, C₁₋₆alkylcarbonyl, C₃₋₇cycloalkylcarbonyl or C₁₋₆alkylphenylsulfonyl; L₄ is S(O)₂ or S(O)(NH); R⁸ is C₁₋₆alkyl, C₃₋₇cycloalkyl; R⁹ is halogen; R¹⁰ is C₁₋₆alkyl, C₃₋₇cycloalkyl; R¹¹ is —NH, —NHCH₃ or —N(CH₃)₂; Cy is pyridyl or chloropyridyl.
   The base in step (a), (c) or (f), can be for example, TEA or DIPEA;
   The oxidate in step (d), can be for example, m-CPBA or PhI(OAc)₂;
   The catalysts in step (e), can be for example, cuprous iodide or L-Proline;
   The base in step (e), can be for example, K₂CO₃;
   The coupling reagent in step (f), can be for example, HATU or T₃P;

A compound of formula (I) when manufactured according to the above process is also an object of the invention.

The compound of this invention also shows good safety and PK profile.

### Pharmaceutical Compositions and Administration

The invention also relates to a compound of formula (I) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduction of HBsAg and HBeAg in HBV patients. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a pharmaceutically acceptable salt thereof.

In a further embodiment includes a pharmaceutical composition comprising a compound of formula (I), or a pharmaceutically acceptable salt thereof, together with a pharmaceutically acceptable carrier or excipient.

Another embodiment includes a pharmaceutical composition comprising a compound of formula (I), or a pharmaceutically acceptable salt thereof for use in the treatment of HBV infection.

### Indications and Methods of Treatment

The compounds of the invention have anti-HBV activity. Accordingly, the compounds of the invention are useful for the treatment or prophylaxis of HBV infection.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for use in the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of formula (I), or a pharmaceutically acceptable salt thereof.

The invention relates in particular to a compound of formula (I) for use in the treatment or prophylaxis of HBV infection.

### EXAMPLES

The invention will be more fully understood by reference to the following examples. They should not, however, be construed as limiting the scope of the invention.

### Abbreviations

ACN: acetonitrile
BINAP: (±)-2,2′-bis(diphenylphosphino)-1,1′-binaphthalene
DIAD: diisopropyl azodiformate
DIPEA: N,N-diisopropylethylamine
DMAP: 4-dimethylaminopyridine
DMF: N,N-dimethylformamide
DMP: Dess-Martin periodinane
DMSO-d₆: deuterated dimethylsulfoxide
DCM: dichloromethane
EC₅₀: the molar concentration of an agonist, which produces 50% of the maximum possible response for that agonist.
EDCI: N-ethyl-N′-(3-dimethylaminopropyl)carbodiimide hydrochloride
EtOAc: ethyl acetate
FBS: fetal bovine serum
h: hour
HATU O-(7-aza-1H-benzotriazole-1-yl)-N,N,N′,N′-tetramethyluronium hexafluorophosphate
HPLC: high performance liquid chromatography
IC₅₀: the half maximal inhibitory concentration
LC/MS: liquid chromatography/mass spectrometry
LDA: lithium diisopropylamide
LiNTf₂: bis(trifluoromethylsulfonyl)amine lithium salt
m-CPBA: 3-chloroperbenzoic acid
MeOH: methanol
MHz: megahertz
min: minute
mL: milliliter
mmol: millimole
MMTrCl: 4-methoxytriphenylmethyl chloride
MsCl: methanesulfonyl chloride
MS (ESI): mass spectroscopy (electron spray ionization)
MTBE: methyl tert-butyl ether
N: mol/L
n-BuLi: n-butyllithium
NMR: nuclear magnetic resonance
obsd.: observed
Pd/C: palladium on carbon
Pd₂(dba)₃: tris(dibenzylideneacetone)dipalladium
PE: petroleum ether
psi: pounds per square inch
SFC: supercritical fluid chromatography
TBAF: tetrabutylammonium fluoride
TEA: triethylamine
TFA: trifluoroacetic acid
TMSOTf: trimethylsilyl trifluoromethanesulfonate
THF: tetrahydrofuran
TLC: thin layer chromatography
T₃P: 2,4,6-tripropyl-1,3,5,2,4,6-trioxatriphosphinane 2,4,6-trioxide
μL: microliter
v/v: volume ratio
xantaphos: 9,9-dimethyl-4,5-bis(diphenylphosphino)xanthene
δ: chemical shift

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

Chiral Separation was conducted on Thar 350 preparative SFC using ChiralPak AD-10p (200×50 mm I.D.) with mobile phase A for CO₂ and B for ethanol. LC/MS spectra were obtained using a Waters UPLC-SQD Mass. Standard LC/MS conditions were as follows (running time: 3 minutes):

Acidic condition: A: 0.1% formic acid and 1% acetonitrile in H₂O; B: 0.1% formic acid in acetonitrile;

Basic condition: A: 0.05% NH₃H₂O in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

### Intermediate 1

### 6-(5-Chloro-1,3-benzoxazol-2-yl)spiro[3.3]heptan-2-amine

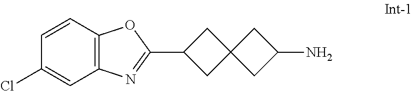 [SMILES: NC1CC2(C1)CC(c1nc3cc(Cl)ccc3o1)C2]

### Preparation of 6-(5-chloro-1,3-benzoxazol-2-yl)spiro[3.3]heptan-2-amine

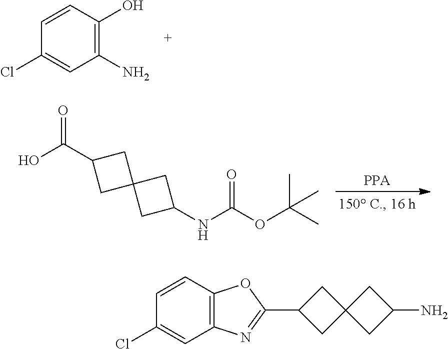

A mixture of 2-(tert-butoxycarbonylamino)spiro[3.3]heptane-6-carboxylic acid (355 mg, 1.39 mmol) and 2-amino-4-chlorophenol (200 mg, 1.39 mmol) in polyphosphoric acid (3.0 mL) was stirred at 180° C. for 2 h. LC-MS showed the reaction was complete. After cooling, the reaction mixture was poured into 4 N NH₄OH (50 mL) with vigorous stirring. The formed precipitate was collected by filtration. The filter cake was triturated in ACN (10 mL), dried in vacuum to afford 6-(5-chloro-1,3-benzoxazol-2-yl)spiro[3.3]heptan-2-amine (Int-1) (300 mg) as a yellow solid.

MS obsd. (ESI⁺) [(M+H)⁺]: 263.0. ¹H NMR (CDCl₃, 400 MHz) δ: 7.64 (d, J=2.0 Hz, 1H), 7.38 (d, J=8.6 Hz, 1H), 7.23-7.30 (m, 1H), 3.65 (quin, J=8.6 Hz, 1H), 3.39 (quin, J=7.8 Hz, 1H), 2.45-2.63 (m, 4H), 2.32-2.45 (m, 2H), 1.83 (dd, J=11.0, 8.3 Hz, 1H), 1.72-1.79 (m, 1H).

### Intermediate 2

### 6-Oxazolo[4,5-b]pyridin-2-ylspiro[3.3]heptan-2-amine

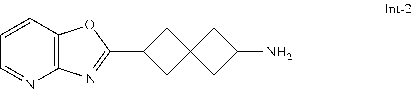 [SMILES: NC1CC2(C1)CC(c1nc3ncccc3o1)C2]

The title compound was prepared in analogy to the procedure described for the preparation of Int-1, by using 2-amino-3-hydroxypyridine instead of 2-amino-4-chlorophenol to afford Int-2 as a brown solid.

MS obsd. (ESI⁺) [(M+H)⁺]: 230.2.

### Intermediate 3

### 6-(6-Chlorooxazolo[5,4-b]pyridin-2-yl)spiro[3.3]heptan-2-amine

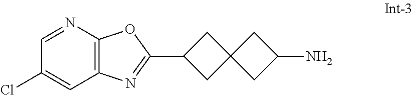 [SMILES: NC1CC2(C1)CC(c1nc3cc(Cl)cnc3o1)C2]

### Preparation of tert-butyl N-[6-[(5-chloro-2-hydroxy-3-pyridyl)carbamoyl]spiro[3.3]heptan-2-yl]carbamate

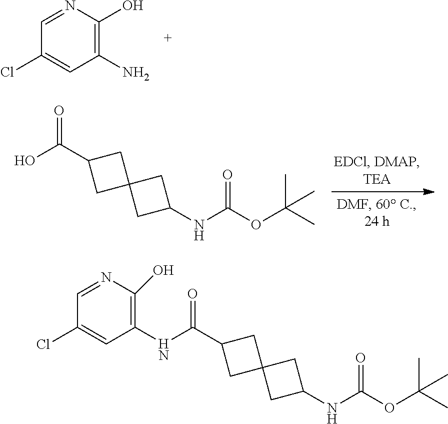

To a solution of 2-(tert-butoxycarbonylamino)spiro[3.3]heptane-6-carboxylic acid (10.0 g, 39.2 mmol) in DMF (150 mL) was added 3-amino-5-chloro-pyridin-2-ol (5.95 g, 41.1 mmol), EDCI (11.26 g, 58.8 mmol), DMAP (7.18 g, 58.8 mmol) and TEA (16.3 mL, 117.5 mmol). Then the reaction mixture was stirred at 60° C. for 24 h. The reaction was washed with water (400 mL×3), extracted with DCM (500 mL×2). The organic layers were washed with brine (600 mL), dried over Na₂SO₄ and concentrated in vacuum. The residue was purified by silica gel column eluted with DCM/MeOH=50/1 to DCM/MeOH=30/1 to give tert-butyl N-[6-[(5-chloro-2-hydroxy-3-pyridyl)carbamoyl]spiro[3.3]heptan-2-yl]carbamate (10.0 g) as an off-white solid. MS obsd. (ESI⁺) [(M+Na)⁺]: 404.1.

### Preparation of tert-butyl N-[6-(6-chlorooxazolo[5,4-b]pyridin-2-yl)spiro[3.3]heptan-2-yl]carbamate

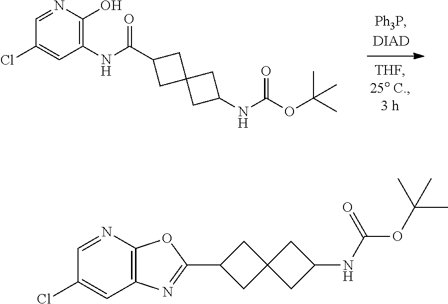 [SMILES: CC(C)(C)OC(=O)NC1CC2(C1)CC(C(=O)Nc1cc(Cl)cnc1O)C2 ; CC(C)(C)OC(=O)NC1CC2(C1)CC(c1nc3cc(Cl)cnc3o1)C2]

A mixture of tert-butyl N-[6-[(5-chloro-2-hydroxy-3-pyridyl)carbamoyl]spiro[3.3]heptan-2-yl]carbamate (5.0 g, 13.1 mmol), triphenylphosphine (5.15 g, 19.6 mmol) and DIAD (3.87 mL, 19.6 mmol) in THF (20 mL) was stirred at 25° C. for 3 h. The mixture was concentrated in vacuum and the residue was purified by silica gel column eluted with PE/EtOAc=1/1 to give tert-butyl N-[6-(6-chlorooxazolo[5,4-b]pyridin-2-yl)spiro[3.3]heptan-2-yl]carbamate (4.0 g) as a white solid.

MS obsd. (ESI⁺) [(M+Na)⁺]: 386.1.

### Preparation of 6-(6-chlorooxazolo[5,4-b]pyridin-2-yl)spiro[3.3]heptan-2-amine

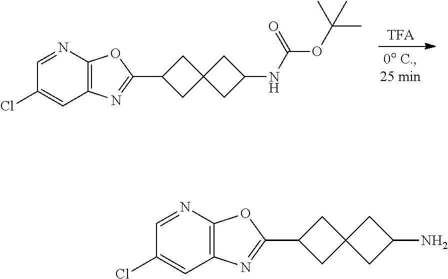 [SMILES: CC(C)(C)OC(=O)NC1CC2(C1)CC(c1nc3cc(Cl)cnc3o1)C2 ; NC1CC2(C1)CC(c1nc3cc(Cl)cnc3o1)C2]

A solution of tert-butyl N-[6-(6-chlorooxazolo[5,4-b]pyridin-2-yl)spiro[3.3]heptan-2-yl]carbamate (500.0 mg, 1.37 mmol) in TFA (2.0 mL, 1.37 mmol) was stirred at 0° C. for 25 min. The mixture was quenched with saturated aqueous Na₂CO₃ and extracted with DCM (20 mL×3). The organic layers were dried over Na₂SO₄ and concentrated in vacuum to afford 6-(6-chlorooxazolo[5,4-b]pyridin-2-yl)spiro[3.3]heptan-2-amine (Int-3) (200 mg) as light yellow oil. MS obsd. (ESI⁺) [(M+Na)⁺]: 264.1.

### Intermediate 4

### 6-[5-(Trifluoromethyl)-1,3-benzoxazol-2-yl]spiro[3.3]heptan-2-amine

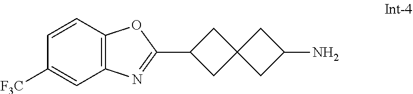 [SMILES: NC1CC2(C1)CC(c1nc3cc(C(F)(F)F)ccc3o1)C2]

The title compound was prepared in analogy to the procedure described for the preparation of Int-3, by using 2-amino-4-(trifluoromethyl)phenol instead of 3-amino-5-chloro-pyridin-2-ol to afford Int-4 as yellow oil.

MS obsd. (ESI⁺) [(M+H)⁺]: 297.1.

### Intermediate 5

### 2-(Oxetan-3-ylamino)pyridine-4-carboxylic acid

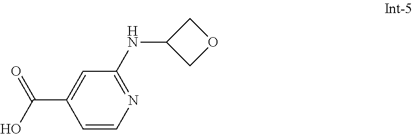 [SMILES: O=C(O)c1ccnc(NC2COC2)c1]

### Preparation of methyl 2-(oxetan-3-ylamino)pyridine-4-carboxylate

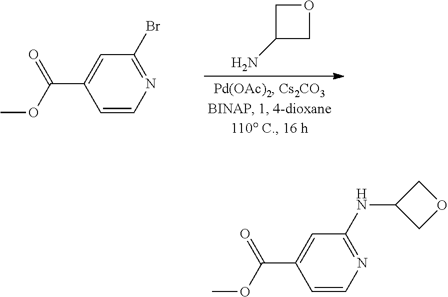

To a solution of methyl 2-bromopyridine-4-carboxylate (200 mg, 0.93 mmol) in 1,4-dioxane (6 mL) was added oxetan-3-amine (81 mg, 1.11 mmol), Cs₂CO₃ (904 mg, 2.78 mmol) and BINAP (115 mg, 0.19 mmol), then the reaction was purged with N₂ for three times. Pd(OAc)₂ (20 mg, 0.090 mmol) was added, then the reaction was stirred at 110° C. for 16 h. The reaction was concentrated in vacuum to give the crude product methyl 2-(oxetan-3-ylamino)pyridine-4-carboxylate (240 mg) as brown oil.

MS obsd. (ESI⁺) [(M+H)⁺]: 209.0

### Preparation of methyl 2-(oxetan-3-ylamino)pyridine-4-carboxylate

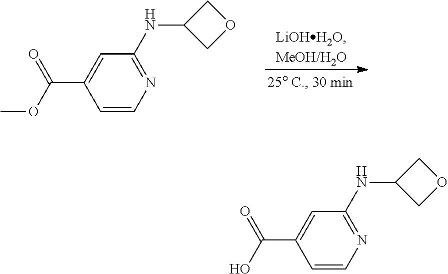

To a solution of methyl 2-(oxetan-3-ylamino)pyridine-4-carboxylate (240 mg, 1.15 mmol) in a mixed solution of methanol (4 mL) and water (2 mL) was added LiOH·H₂O (242 mg, 5.76 mmol). Then the reaction mixture was stirred at 25° C. for 30 min. The reaction mixture was washed with DCM (10 mL×2) and the aqueous layer was acidified by 1 N HCl solution to pH=7. The resulting mixture was lyophilized to give 2-(oxetan-3-ylamino)pyridine-4-carboxylic acid (Int-5) (300 mg) as a yellow solid. The crude product was used for next step without further purification.

MS obsd. (ESI⁺) [(M+H)⁺]: 195.0

### Intermediate 6

### 2-(Diethylamino)pyridine-4-carboxylic acid

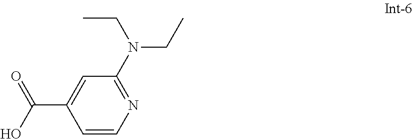

The title compound was prepared in analogy to the procedure described for the preparation of Int-5, by using diethylamine instead of oxetan-3-amine to afford Int-6 as a white solid. MS obsd. (ESI⁺) [(M+H)⁺]: 195.1

### Intermediate 7

### 2-[Ethyl(methyl)amino]pyridine-4-carboxylic acid

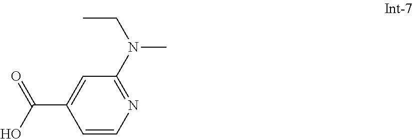

The title compound was prepared in analogy to the procedure described for the preparation of Int-5, by using N-ethylmethylamine instead of oxetan-3-amine to afford Int-7 as a light yellow solid.

MS obsd. (ESI⁺) [(M+H)⁺]: 181.1

### Intermediate 8

### 2-(1-Piperidyl)pyridine-4-carboxylic acid

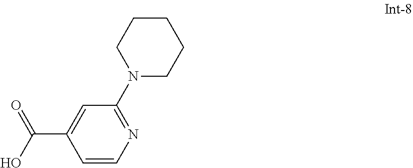 [SMILES: O=C(O)c1ccnc(N2CCCCC2)c1]

### Preparation of 2-(1-piperidyl)pyridine-4-carboxylic acid

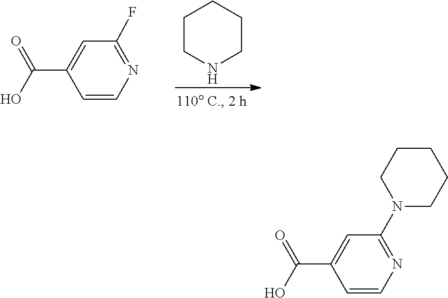 [SMILES: O=C(O)c1ccnc(F)c1 ; C1CCNCC1 ; O=C(O)c1ccnc(N2CCCCC2)c1]

A mixture of 2-fluoroisonicotinic acid (1 g, 7.09 mmol) in piperidine (10.0 mL) was heated at 110° C. for 2 h. After cooling to room temperature, the reaction was concentrated to dryness and the residue was dissolved in DCM (100 mL) and 1 N HCl was added to adjust pH=6. The separated organic phase was washed with water (50 mL), bine (50 mL) and dried over Na₂SO₄. The solution was concentrated in vacuum to give 2-(1-piperidyl)pyridine-4-carboxylic acid (Int-8) (1.4 g) as a yellow solid.

MS obsd. (ESI⁺) [(M+H)⁺]: 207.2

### Intermediate 9

### 2-Morpholinopyridine-4-carboxylic acid

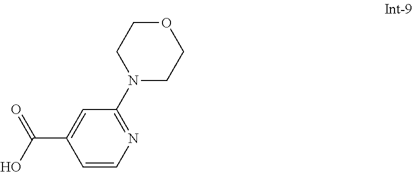 [SMILES: O=C(O)c1ccnc(N2CCOCC2)c1]

The title compound was prepared in analogy to the procedure described for the preparation of Int-5, by using morpholine instead of oxetan-3-amine to afford Int-9 as light yellow oil.

MS obsd. (ESI⁺) [(M+H)⁺]: 209.2

### Intermediate 10

### 2-(Cyclopropylamino)pyridine-4-carboxylic acid

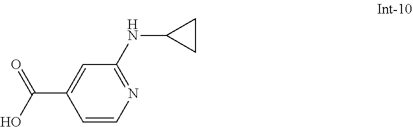 [SMILES: O=C(O)c1ccnc(NC2CC2)c1]

The title compound was prepared in analogy to the procedure described for the preparation of Int-5, by using cyclopropanamine instead of oxetan-3-amine to afford Int-10 as a white solid.

MS obsd. (ESI⁺) [(M+H)⁺]: 179.1

### Intermediate 11

### 2-[Isopropyl(methyl)amino]pyridine-4-carboxylic acid

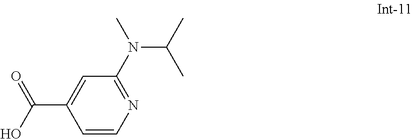

The title compound was prepared in analogy to the procedure described for the preparation of Int-5, by using N-methylpropan-2-amine instead of oxetan-3-amine to afford Int-11 as a white solid.

MS obsd. (ESI⁺) [(M+H)⁺]: 195.1

### Intermediate 12

### 2-(2-Oxopyrrolidin-1-yl)pyridine-4-carboxylic acid

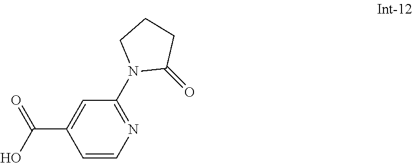 [SMILES: O=C(O)c1ccnc(N2CCCC2=O)c1]

The title compound was prepared in analogy to the procedure described for the preparation of Int-5, by using pyrrolidin-2-one instead of oxetan-3-amine to afford Int-12 as a yellow solid.

MS obsd. (ESI⁺) [(M+H)⁺]: 207.1

### Intermediate 13

### 2-(4-Methylthiazol-2-yl)pyridine-4-carboxylic acid

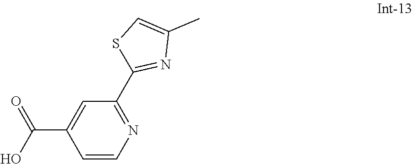

The title compound was prepared in analogy to the procedure described for the preparation of Int-5, by using 2-bromo-4-methyl-thiazole instead of oxetan-3-amine to afford Int-13 as a light yellow solid.

MS obsd. (ESI⁺) [(M+H)⁺]: 221.0

### Intermediate 14

### 2-(5-Methylthiazol-2-yl)pyridine-4-carboxylic acid

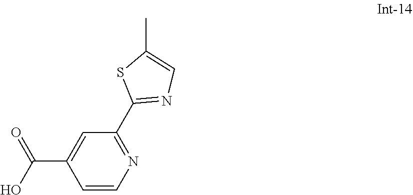

The title compound was prepared in analogy to the procedure described for the preparation of Int-5, by using 2-bromo-5-methyl-thiazole instead of oxetan-3-amine to afford Int-14 as a white solid.

MS obsd. (ESI⁺) [(M+H)⁺]: 221.2

### Intermediate 15

### 2-Thiazol-2-ylpyridine-4-carboxylic acid

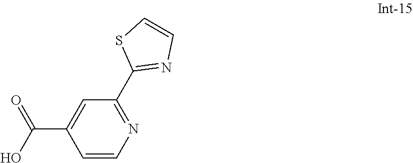 [SMILES: O=C(O)c1ccnc(-c2nccs2)c1]

The title compound was prepared in analogy to the procedure described for the preparation of Int-5, by using 2-(tributylstannyl)thiazole instead of oxetan-3-amine to afford Int-15 as a white solid.

MS obsd. (ESI⁺) [(M+H)⁺]: 207.1

### Intermediate 16

### 2-Isopropoxypyridine-4-carboxylic acid

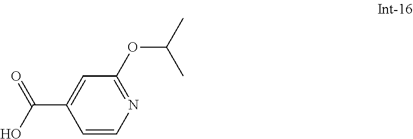

### Preparation of methyl 2-isopropoxypyridine-4-carboxylate

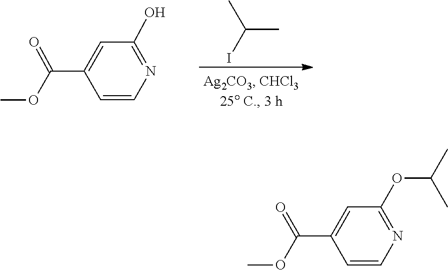

To a solution of methyl 2-hydroxypyridine-4-carboxylate (120 mg, 0.78 mmol) in chloroform (5 mL), then 2-iodopropane (0.31 mL, 3.92 mmol) and silver carbonate (432 mg, 1.57 mmol) was added. The mixture was stirred at 25° C. for 3 h. The mixture was filtered and washed with DCM (10 ml) and dried over Mg₂SO₄. The organic layer was concentrated in vacuum and the residue was purified by silica gel column eluted with PE/EtOAc=10/1 to afford methyl 2-isopropoxypyridine-4-carboxylate (120 mg) as colorless oil.

MS obsd. (ESI⁺) [(M+H)⁺]: 196.1.

### Preparation of 2-isopropoxypyridine-4-carboxylic acid

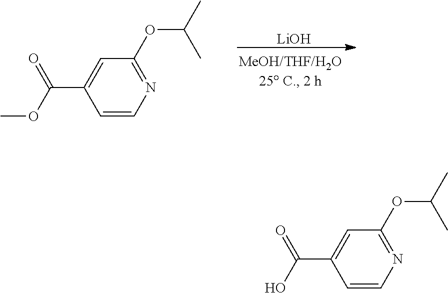

To a solution of methyl 2-isopropoxypyridine-4-carboxylate (129 mg, 0.66 mmol) in THF (3 mL) was added MeOH (3 mL). There after the solution of lithium hydroxide (0.03 mL, 3.31 mmol) was added and the mixture was stirred at 25° C. for 2 h. The reaction solution was concentrated in vacuum to afford 2-isopropoxypyridine-4-carboxylic acid (Int-16) (100 mg) as a light yellow solid. The crude was used for next step without further purification.

MS obsd. (ESI⁺) [(M+H)⁺]: 182.2.

### Intermediate 17

### 2-(Methylsulfamoylamino)pyridine-4-carboxylic acid

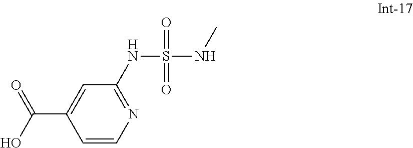

### Preparation of ethyl 2-(methylsulfamoylamino)pyridine-4-carboxylate

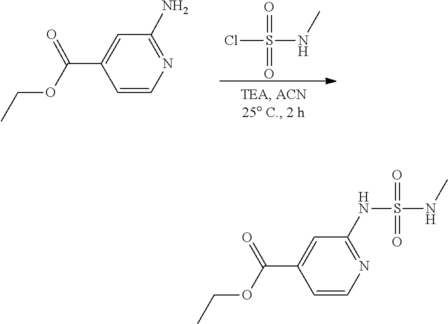

To a solution of ethyl 2-aminopyridine-4-carboxylate (200 mg, 1.2 mmol) in ACN (4 mL) was added N-methyl sulfamoyl chloride (155 mg, 1.20 mmol) and TEA (0.33 mL, 2.41 mmol). Then the reaction was stirred at 25° C. for 2 h. The reaction mixture was concentrated to give the crude, which was purified by silica gel column eluted with PE/EtOAc=20/1 to 3/1 to give ethyl 2-(methylsulfamoylamino)pyridine-4-carboxylate (250 mg) as a light yellow solid.

MS obsd. (ESI⁺) [(M+H)⁺]: 260.0.

### Preparation of 2-(methylsulfamoylamino)pyridine-4-carboxylic acid

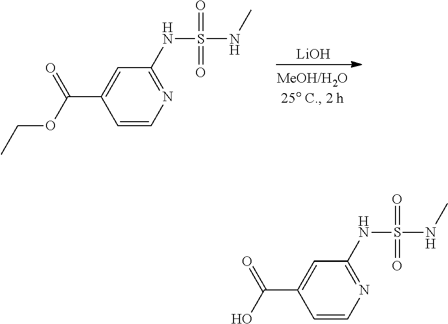 [SMILES: CCOC(=O)c1ccnc(NS(=O)(=O)NC)c1 ; "LiOH" ; CNS(=O)(=O)Nc1cc(C(=O)O)ccn1]

To a solution of ethyl 2-(methylsulfamoylamino)pyridine-4-carboxylate (250 mg, 0.96 mmol) in a mixed solution of methanol (3.2 mL) and water (0.8 ml) was added LiOH (46 mg, 1.93 mmol). Then the reaction was stirred at 25° C. for 2 h and concentrated in vacuum. The residue was diluted with water (5 ml) and washed with DCM (10 mL×2). The aqueous layer was acidified by 2 N HCl solution to pH=3, then extracted with EtOAc (10 mL×2). The organic layers were concentrated to afford 2-(methylsulfamoylamino)pyridine-4-carboxylic acid (Int-17) (170 mg) as a white solid.

MS obsd. (ESI⁺) [(M+H)⁺]: 232.0.

### Intermediate 18

### 5-Methylsulfonylpyridine-3-carboxylic acid

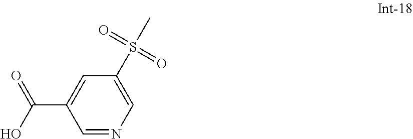

### Preparation of methyl 5-methylsulfonylpyridine-3-carboxylate

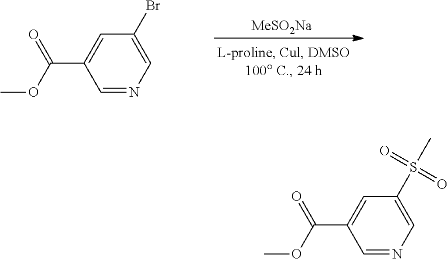

A mixture of methyl 5-bromopyridine-3-carboxylate, CuI (38 mg, 0.200 mmol), methylsulfinyloxysodium (204 mg, 2.00 mmol) and L-proline (23 mg, 0.20 mmol) in DMSO (2 mL) was stirred at 100° C. for 24 h. The reaction mixture was diluted with DCM (30 ml). The resulting mixture was washed with brine (30 mL×3), dried over Na₂SO₄ and concentrated in vacuum. The residue was purified by silica gel column eluted with PE/EtOAc=3/1 to give methyl 5-methylsulfonylpyridine-3-carboxylate (80 mg) as light yellow oil.

MS obsd. (ESI⁺) [(M+H)⁺]: 216.1.

### Preparation of 5-methylsulfonylpyridine-3-carboxylic acid

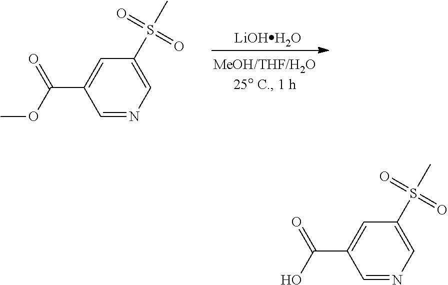

To a solution of methyl 5-methylsulfonylpyridine-3-carboxylate (80 mg, 0.37 mmol) in a mixed solution of MeOH (0.5 mL) and THF (0.5 mL) was added a solution of LiOH·H₂O (31 mg, 0.74 mmol) in water (0.5 mL). The mixture was stirred at 25° C. for 1 h. The mixture was concentrated in vacuum to give the 5-methylsulfonylpyridine-3-carboxylic acid (Int-18) (74 mg) as a white solid. The crude product was used for next step directly without further purification.

MS obsd. (ESI⁺) [(M+H)⁺]: 202.0.

### Intermediate 19

### 2-(Oxetan-3-ylsulfonyl)pyridine-3-carboxylic acid

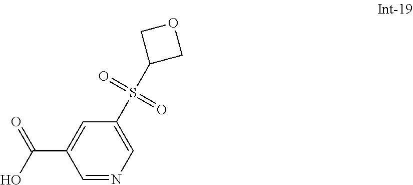 [SMILES: O=C(O)c1cncc(S(=O)(=O)C2COC2)c1]

### Preparation of methyl 2-(oxetan-3-ylsulfanyl)pyridine-4-carboxylate

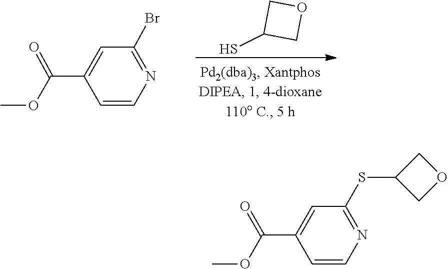

To a solution of oxetane-3-thiol (83 mg, 0.93 mmol) in 1,4-dioxane (5 mL) was added methyl 2-bromopyridine-4-carboxylate (200 mg, 0.93 mmol), xantphos (107 mg, 0.19 mmol) and DIPEA (0.48 mL, 2.78 mmol). Then the reaction mixture was purged with N₂ for three times. Pd₂(dba)₃ (84 mg, 0.090 mmol) was added to the reaction mixture and then the reaction was stirred at 110° C. for 5 h. The reaction mixture was concentrated to give the crude, which was purified by silica gel column eluted with PE to PE/EtOAc=5/1 to afford methyl 2-(oxetan-3-ylsulfanyl)pyridine-4-carboxylate (240 mg) as light brown oil.

MS obsd. (ESI⁺) [(M+H)⁺]: 226.0.

### Preparation of methyl 2-(oxetan-3-ylsulfonyl)pyridine-4-carboxylate

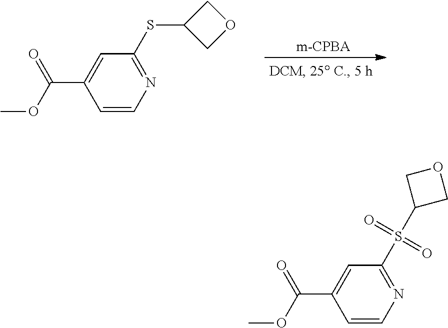 [SMILES: COC(=O)c1ccnc(SC2COC2)c1 ; COC(=O)c1ccnc(S(=O)(=O)C2COC2)c1]

To a solution of methyl 2-(oxetan-3-ylsulfanyl)pyridine-4-carboxylate (240 mg, 1.07 mmol) in DCM (19 mL) was added m-CPBA (367 mg, 2.13 mmol). Then the solution was stirred at 25° C. for 4 h. The reaction was quenched with saturated aqueous Na₂S₂O₃ and extracted with DCM (30 mL×2). The organic layers were concentrated to give the crude product, which was purified by silica gel column eluted with PE/EtOAc=10/1 to 1/1 to give methyl 2-(oxetan-3-ylsulfonyl)pyridine-4-carboxylate (260 mg) as a white solid.

MS obsd. (ESI⁺) [(M+H)⁺]: 258.0.

### Preparation of 2-(oxetan-3-ylsulfonyl)pyridine-4-carboxylic acid

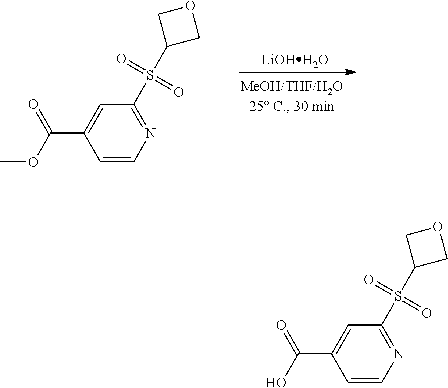 [SMILES: COC(=O)c1ccnc(S(=O)(=O)C2COC2)c1 ; "LiOH·H2O" ; O=C(O)c1ccnc(S(=O)(=O)C2COC2)c1]

To a solution of methyl 2-(oxetan-3-ylsulfonyl)pyridine-4-carboxylate (260 mg, 1.01 mmol) in a mixed solution of MeOH (6 mL) and water (3 mL) was added LiOH·H₂O (214 mg, 5.05 mmol). The reaction was stirred at 25° C. for 30 min. The reaction mixture was washed with DCM (30 mL×2). The aqueous layer was acidified by 2 N HCl to pH=3 and extracted with EtOAc (30 mL×2). The organic layers were combined and concentrated to give 2-(oxetan-3-ylsulfonyl)pyridine-4-carboxylic acid (Int-19) (220 mg) as a white solid.

MS obsd. (ESI⁺) [(M+H)⁺]: 244.0.

### Intermediate 20

### 2-Ethylsulfonylpyridine-4-carboxylic acid

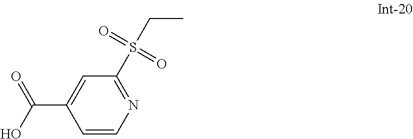

The title compound was prepared in analogy to the procedure described for the preparation of Int-19, by using sodium ethanethiolate instead of oxetane-3-thiol to afford Int-20 as a white solid.

MS obsd. (ESI⁺) [(M+H)⁺]: 216.1

### Intermediate 21

### 1-Tert-butyl-5-oxo-pyrrolidine-3-carboxylic acid

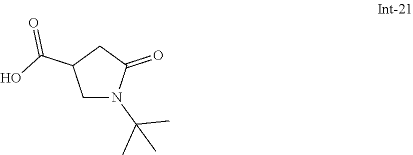

### Preparation of methyl 1-tert-butyl-5-oxo-pyrrolidine-3-carboxylate

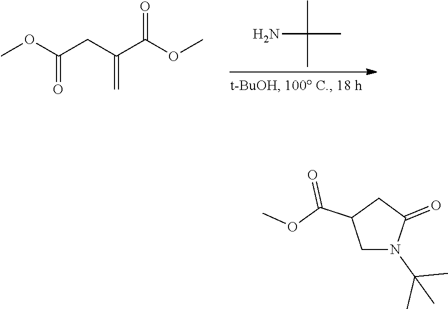

To a solution of dimethyl 2-methylenebutanedioate (500 mg, 3.16 mmol) in t-BuOH (10 mL) was added tert-butylamine (2312 mg, 31.6 mmol). After the addition, the mixture was stirred at 100° C. for 18 h. Then the reaction mixture was concentrated in vacuum to afford methyl 1-tert-butyl-5-oxo-pyrrolidine-3-carboxylate (500 mg) as colorless oil which was used for next step without further purification.

MS obsd. (ESI⁺) [(M+H)⁺]: 200.1.

### Preparation of 1-tert-butyl-5-oxo-pyrrolidine-3-carboxylic acid

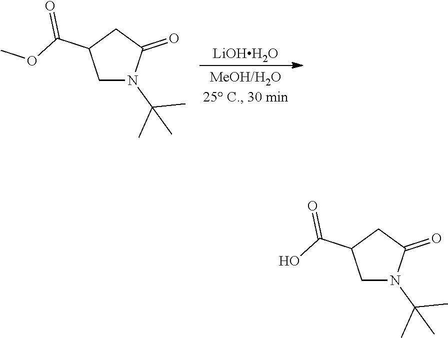

To a solution of methyl 1-tert-butyl-5-oxo-pyrrolidine-3-carboxylate (400.0 mg, 2.01 mmol) in a mixed solution of MeOH (6 mL) and water (3 mL) was added LiOH·H₂O (842 mg, 20.1 mmol), then the reaction was stirred at 25° C. for 2 h. The reaction mixture was washed with DCM (30 mL×2). Then the aqueous layer was acidified by 2 N HCl to pH=3 and extracted with EtOAc (30 mL×2). The organic layers were combined and concentrated to give 1-tert-butyl-5-oxo-pyrrolidine-3-carboxylic acid (Int-21) (100 mg) as colorless oil.

MS obsd. (ESI⁺) [(M+H)⁺]: 186.2.

### Intermediate 22

### 5-Oxo-1-phenyl-pyrrolidine-3-carboxylic acid

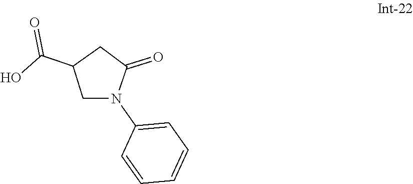 [SMILES: O=C(O)C1CC(=O)N(c2ccccc2)C1]

### Preparation of methyl 5-oxo-1-phenyl-pyrrolidine-3-carboxylic acid

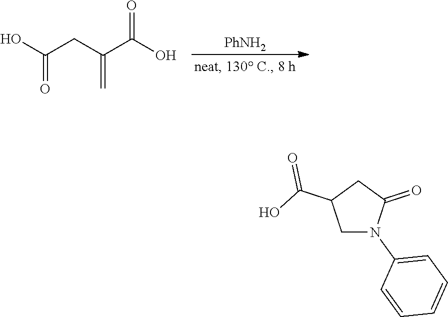

A mixture of 2-methylenebutanedioic acid (0.25 mL, 3.07 mmol) and aniline (0.28 mL, 3.07 mmol) was heated at 130° C. for 8 h. The mixture was concentrated in vacuum to give 5-oxo-1-phenyl-pyrrolidine-3-carboxylic acid (Int-22) (400 mg) as a brown solid. The crude product was used for next step without further purification.

MS obsd. (ESI⁺) [(M+H)⁺]: 206.2.

### Intermediate 23

### 1-(3-Chloro-2-methyl-phenyl)-5-oxo-pyrrolidine-3-carboxylic acid

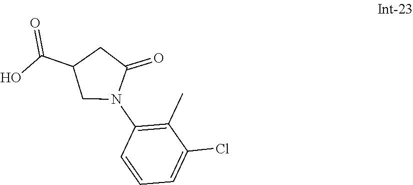 [SMILES: Cc1c(Cl)cccc1N1CC(C(=O)O)CC1=O]

The title compound was prepared in analogy to the procedure described for the preparation of Int-22, by using 3-chloro-2-methyl-aniline instead of aniline to afford Int-23 as a dark brown solid. MS obsd. (ESI⁺) [(M+H)⁺]: 254.1

### Intermediate 24

### 1,1-Dioxo-1,2-thiazolidine-4-carboxylic acid

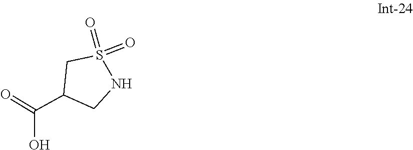

### Preparation of 1-(benzylamino)-3-benzyloxy-propan-2-ol

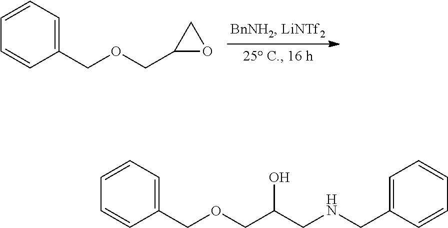 [SMILES: c1ccc(COCC2CO2)cc1 ; OC(CNCc1ccccc1)COCc1ccccc1]

To a solution of benzyl glycidyl ether (10.0 g, 60.9 mmol) in DCM (100 mL) was added benzylamine (5.99 mL, 54.8 mmol) and LiNTf₂ (1.75 g, 6.09 mmol). Then the reaction mixture was stirred at 25° C. for 16 h. The reaction mixture was concentrated to give the crude, which was purified by silica gel column eluted with DCM to DCM/MeOH=10/1 to give 1-(benzylamino)-3-benzyloxy-propan-2-ol (13.0 g, 47.9 mmol) as colorless oil. MS obsd. (ESI⁺) [(M+H)⁺]: 272.1.

### Preparation of [1-[[benzyl(methylsulfonyl)amino]methyl]-2-benzyloxy-ethyl]methanesulfonate

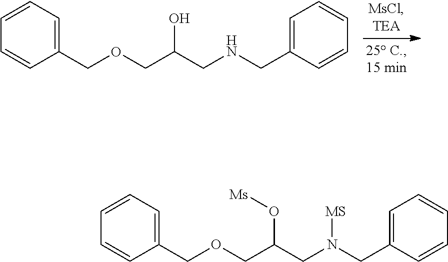

To a solution of 1-(benzylamino)-3-benzyloxy-propan-2-ol (12.0 g, 44.2 mmol) in DCM (120 mL) was added TEA (18.44 mL, 132.6 mmol). Then MsCl (10.27 mL, 132.6 mmol) was added dropwise. The reaction was stirred at 25° C. for 15 min and quenched with saturated aqueous NaHCO₃ (100 mL). The resulting mixture was extracted with EtOAc (100 mL×2), washed with brine (200 mL), dried over Na₂SO₄ and concentrated in vacuum. The residue was purified by silica gel column eluted with PE to PE/EtOAc=3/1 to give [1-[[benzyl(methylsulfonyl)amino]methyl]-2-benzyloxy-ethyl] methanesulfonate (16.9 g) as a white solid.

MS obsd. (ESI⁺) [(M+Na)⁺]: 450.1. ¹H NMR (CDCl₃, 400 MHz) δ: 7.44-7.21 (m, 10H), 4.95-4.79 (m, 1H), 4.60 (d, J=15.1 Hz, 1H), 4.45 (dt, J=18.6, 13.4 Hz, 3H), 3.65-3.35 (m, 4H), 3.01 (s, 3H), 2.90 (d, J=5.3 Hz, 3H).

### Preparation of 2-benzyl-4-(benzyloxymethyl)-1,2-thiazolidine 1,1-dioxide

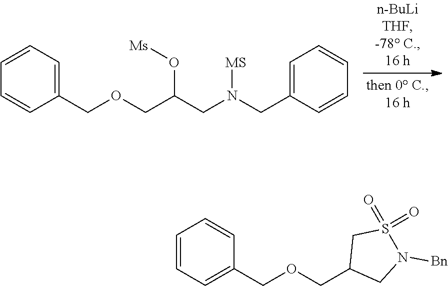

To a solution of [1-[[benzyl(methylsulfonyl)amino]methyl]-2-benzyloxy-ethyl]methanesulfonate (5.0 g, 11.7 mmol) in THF (144 mL) at −78° C. was added n-BuLi (12.2 mL, 29.2 mmol) dropwise under nitrogen atmosphere. Then the reaction mixture was stirred at 0° C. for 16 h and quenched with saturated aqueous NH₄Cl. The mixture was extracted with EtOAc (50 mL×2). The organic layers were concentrated in vacuum and the residue was purified by silica gel column eluted with PE to PE/EtOAc=3/1 to afford 2-benzyl-4-(benzyloxymethyl)-1,2-thiazolidine 1,1-dioxide (1.71 g) as colorless oil.

MS obsd. (ESI⁺) [(M+Na)⁺]: 332.1.

### Preparation of (1,1-dioxo-1,2-thiazolidin-4-yl)methanol

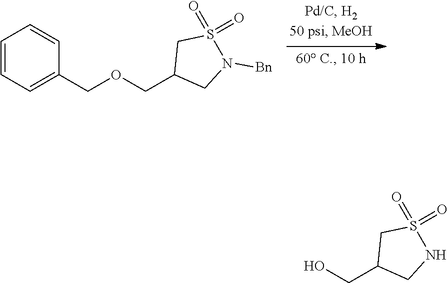 [SMILES: O=S1(=O)CC(COCc2ccccc2)CN1Cc1ccccc1 ; O=S1(=O)CC(CO)CN1]

To a solution of 2-benzyl-4-(benzyloxymethyl)-1,2-thiazolidine 1,1-dioxide (4.27 g, 12.9 mmol) in MeOH (20 mL) was added Pd/C (13.71 g, 12.9 mmol). The reaction mixture was stirred at 60° C. for 10 h under 50 psi of hydrogen atmosphere. The reaction mixture was filtered through a celite pad and the filtrate was concentrated. The residue was purified by silica gel column eluted with DCM to DCM/MeOH=4/1 to afford (1,1-dioxo-1,2-thiazolidin-4-yl)methanol (830 mg) as colorless oil.

MS obsd. (ESI⁺) [(M+Na)⁺]: 152.0.

### Preparation of 1,1-dioxo-1,2-thiazolidine-4-carbaldehyde

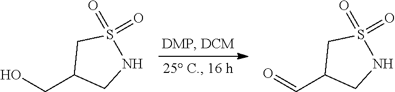 [SMILES: O=S1(=O)CC(CO)CN1 ; O=CC1CNS(=O)(=O)C1]

To a solution of (1,1-dioxo-1,2-thiazolidin-4-yl)methanol (700 mg, 4.63 mmol) in DCM (15 mL) was added DMP (2.9 g, 6.95 mmol), then the reaction mixture was stirred at 25° C. for 16 h. The reaction mixture was washed with saturated aqueous NaHCO₃ and water (50 mL), extracted with DCM (50 mL×2). LC-MS showed the product was dissolved in the aqueous solution. The aqueous phase was combined and lyophilized to give the crude product 1,1-dioxo-1,2-thiazolidine-4-carbaldehyde (800 mg, crude) as a light yellow solid.

MS obsd. (ESI⁺) [(M+Na)⁺]: 150.0.

### Preparation of 1,1-dioxo-1,2-thiazolidine-4-carboxylic acid

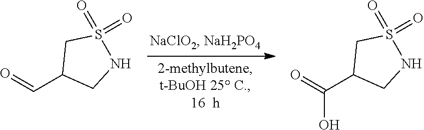 [SMILES: O=CC1CNS(=O)(=O)C1 ; O=C(O)C1CNS(=O)(=O)C1]

To a solution of 1,1-dioxo-1,2-thiazolidine-4-carbaldehyde (800 mg, crude) in t-BuOH (8 mL) was added NaClO₂ (303 mg, 3.35 mmol), NaH₂PO₄ (241 mg, 2.01 mmol) and 2-methylbutene (940 mg, 13.4 mmol). Then the reaction was stirred at 25° C. for 16 h and concentrated to give the crude product, which was dissolved in water and washed with DCM (50 mL). The aqueous phase was combined and acidified by 2 N HCl solution to pH=3, extracted with EtOAc (50 mL×2). The aqueous phase was combined and lyophilized to give the crude product 1,1-dioxo-1,2-thiazolidine-4-carbaldehyde (Int-24) (600 mg) as a light yellow solid.

MS obsd. (ESI⁺) [(M+Na)⁺]: 166.0.

### Intermediate 25

### 2-Benzyl-1,1-dioxo-1,2-thiazolidine-4-carboxylic acid

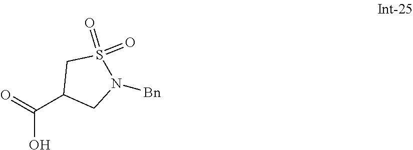 [SMILES: O=C(O)C1CN(Cc2ccccc2)S(=O)(=O)C1]

The title compound was prepared in analogy to the procedure described for the preparation of Int-24, by using Pd/C, 15 psi of H₂ and 25° C. instead of Pd/C, 50 psi of H₂ and 60° C. as the hydrogenation condition to afford Int-25 as a white solid.

MS obsd. (ESI⁺) [(M+Na)⁺]: 256.0.

### Intermediate 26

### 2-Methyl-1,1-dioxo-1,2-thiazolidine-3-carboxylic acid

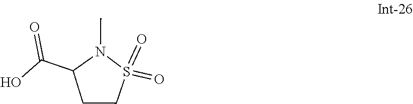

### Preparation of 1-(benzylamino)-3-benzyloxy-propan-2-ol

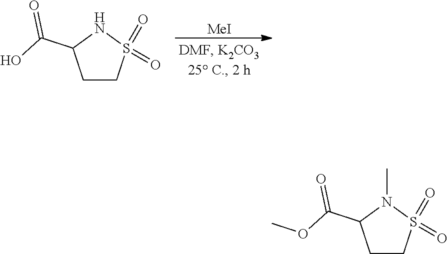

To a solution of 1,1-dioxo-1,2-thiazolidine-3-carboxylic acid (400 mg, 2.42 mmol) in DMF (10 mL) was added K₂CO₃ (1.0 g, 7.27 mmol) and Mel (0.23 mL, 3.63 mmol). Then the reaction mixture was stirred at 25° C. for 2 h. The reaction solution was diluted with DCM (40 ml) and washed with water (20 mL×2). The organic layers were concentrated and the residue was purified by silica gel column eluted with PE to PE/EtOAc=7/1 to give methyl 2-methyl-1,1-dioxo-1,2-thiazolidine-3-carboxylate (150 mg) as yellow oil.

### Preparation of 2-methyl-1,1-dioxo-1,2-thiazolidine-3-carboxylic acid

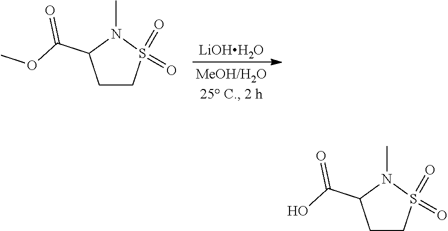

To a solution of methyl 2-methyl-1,1-dioxo-1,2-thiazolidine-3-carboxylate (150 mg, 0.78 mmol) in a mixed solution of methanol (3 mL) and water (1 ml) was added LiOH·H₂O (98 mg, 2.34 mmol). The reaction mixture was stirred at 25° C. for 2 h. The reaction mixture was concentrated in vacuum and the residue was acidified by 2N HCl to pH=3. The resulting mixture was extracted with EtOAc (10 ml×3). The organic layers were dried over Na₂SO₄ and concentrated in vacuum to afford 2-methyl-1,1-dioxo-1,2-thiazolidine-3-carboxylic acid (Int-26) (70 mg) as a white solid.

MS obsd. (ESI⁺) [(M+Na)⁺]: 180.0.

### Intermediate 27

### 5-Chloro-2-(2-isocyanatospiro[3.3]heptan-6-yl)-1,3-benzoxazole

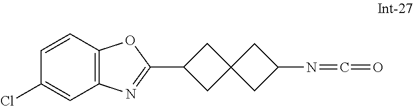 [SMILES: O=C=NC1CC2(C1)CC(c1nc3cc(Cl)ccc3o1)C2]

### Preparation of 5-chloro-2-(2-isocyanatospiro[3.3]heptan-6-yl)-1,3-benzoxazole

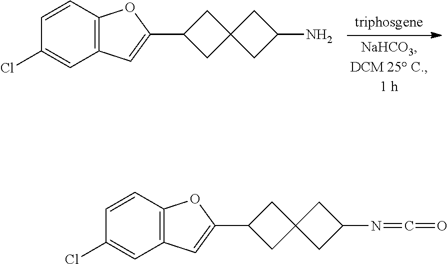 [SMILES: NC1CC2(C1)CC(c1cc3cc(Cl)ccc3o1)C2 ; O=C=NC1CC2(C1)CC(c1cc3cc(Cl)ccc3o1)C2]

To a solution of 6-(5-chloro-1,3-benzoxazol-2-yl)spiro[3.3]heptan-2-amine (Int-1) (1.0 g, 3.81 mmol) in DCM (10 mL) was added NaHCO₃ (3.2 g, 38.1 mmol) at 0° C. Then triphosgene (0.38 g, 1.27 mmol) was added portionwise. The mixture was stirred at 25° C. for 1 h. The mixture was diluted with DCM (90 mL) and washed with water (20 mL×2). The organic layer was dried with Na₂SO₄ and concentrated in vacuum to give 5-chloro-2-(2-isocyanatospiro[3.3]heptan-6-yl)-1,3-benzoxazole (Int-27) (1.09 g) as light yellow oil. The crude was used for next step without further purification.

MS obsd. (ESI⁺) [(M+H)⁺]: 289.0.

### Intermediate 28

### 2-Cyclopropylsulfanylpyridine-4-carboxylic acid

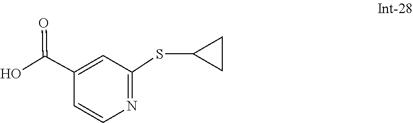 [SMILES: O=C(O)c1ccnc(SC2CC2)c1]

### Preparation of 2-cyclopropylsulfanylpyridine-4-carboxylic acid

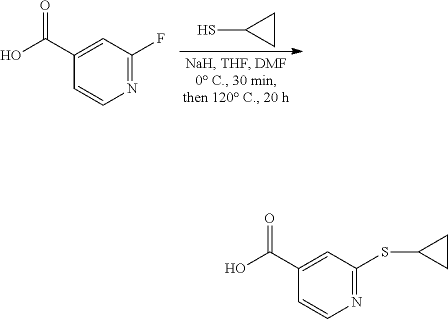

To a solution of cyclopropanethiol (1.57 g, 21.3 mmol) in a mixed solution of THF (40 mL) and DMF (20 mL) was added NaH (170 mg, 7.09 mmol) at 0° C. After stirring at room temperature for 30 min, 2-fluoroisonicotinic acid (1.0 g, 7.09 mmol) was added at 0° C. The reaction was stirred at 120° C. for 20 h. The mixture was filtered and the filtrate was concentrated in vacuum. The residue was purified by pre-HPLC to give 2-cyclopropylsulfanylpyridine-4-carboxylic acid (Int-28) (200 mg) as an off-white solid.

MS obsd. (ESI⁺) [(M+H)⁺]: 196.0.

### Intermediate 29

### 2-Methylsulfanylpyridine-4-carboxylic acid

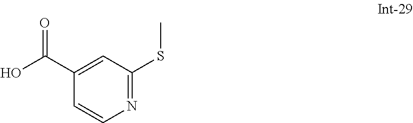

The title compound was prepared in analogy to the procedure described for the preparation of Int-28, by using sodium thiomethoxide instead of cyclopropanethiol to afford Int-29 as a light yellow solid.

MS obsd. (ESI⁺) [(M+H)⁺]: 170.1

### Intermediate 30

### 5-Chloro-2-methylsulfanyl-pyridine-4-carboxylic acid

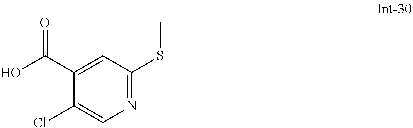

The title compound was prepared in analogy to the procedure described for the preparation of Int-28, by using 2,5-dichloroisonicotinic acid instead of 2-fluoroisonicotinic acid and sodium thiomethoxide instead of cyclopropanethiol to afford Int-30 as a white solid.

MS obsd. (ESI⁺) [(M+H)⁺]: 206.1

### Intermediate 31

### 2-Thiomorpholinopyridine-4-carboxylic acid

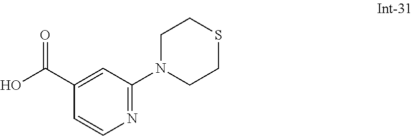 [SMILES: O=C(O)c1ccnc(N2CCSCC2)c1]

The title compound was prepared in analogy to the procedure described for the preparation of Int-5, by using thiomorpholine instead of oxetan-3-amine to afford Int-31 as a white solid. MS obsd. (ESI⁺) [(M+H)⁺]: 225.1

### Intermediate 32

### 2-(1-Tert-butoxycarbonylazetidin-3-yl)sulfonylpyridine-4-carboxylic acid

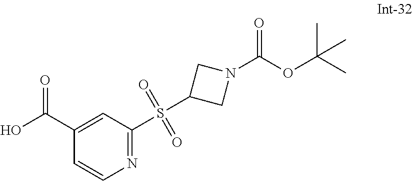 [SMILES: CC(C)(C)OC(=O)N1CC(S(=O)(=O)c2cc(C(=O)O)ccn2)C1]

### Preparation of methyl 2-(1-tert-butoxycarbonylazetidin-3-yl)sulfanylpyridine-4-carboxylate

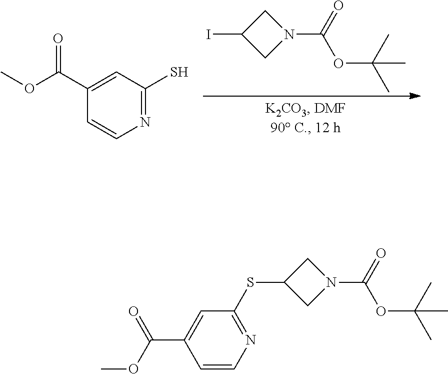 [SMILES: COC(=O)c1ccnc(S)c1 ; CC(C)(C)OC(=O)N1CC(I)C1 ; COC(=O)c1ccnc(SC2CN(C(=O)OC(C)(C)C)C2)c1]

To a solution of methyl 2-sulfanylpyridine-4-carboxylate (3.60 g, 21.3 mmol) and tert-butyl 3-iodoazetidine-1-carboxylate (6.02 g, 21.3 mmol) in DMF (300 mL) was added K₂CO₃ (8.81 g, 63.83 mmol). The reaction mixture was stirred at 90° C. for 12 h. Water (30 ml) was added and the mixture was extracted with EtOAc (200 ml×3). The combined organic layers were dried over Na₂SO₄ and concentrated. The residue was purified by silica gel column eluted with DCM/MeOH=20/1 to afford methyl 2-(1-tert-butoxycarbonylazetidin-3-yl)sulfanylpyridine-4-carboxylate (1.80 g) as a brown solid.

MS obsd. (ESI⁺) [(M+H)⁺]: 325.1

### Preparation of 2-(1-tert-butoxycarbonylazetidin-3-yl)sulfonylpyridine-4-carboxylic acid

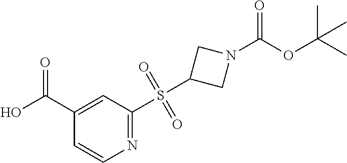 [SMILES: CC(C)(C)OC(=O)N1CC(S(=O)(=O)c2cc(C(=O)O)ccn2)C1]

The title compound was prepared in analogy to the procedure described for the preparation of Int-19, by using 2-(1-tert-butoxycarbonylazetidin-3-yl)sulfanylpyridine-4-carboxylate instead of methyl 2-(oxetan-3-ylsulfanyl)pyridine-4-carboxylate to afford Int-32 as a white solid.

MS obsd. (ESI⁺) [(M+H)⁺]: 343.1

### Example 1

### N-[6-(5-chloro-1,3-benzoxazol-2-yl)spiro[3.3]heptan-2-yl]pyridine-2-carboxamide

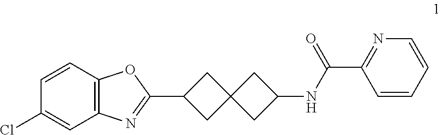 [SMILES: O=C(NC1CC2(C1)CC(c1nc3cc(Cl)ccc3o1)C2)c1ccccn1]

### Preparation of N-[6-(5-chloro-1,3-benzoxazol-2-yl)spiro[3.3]heptan-2-yl]pyridine-2-carboxamide

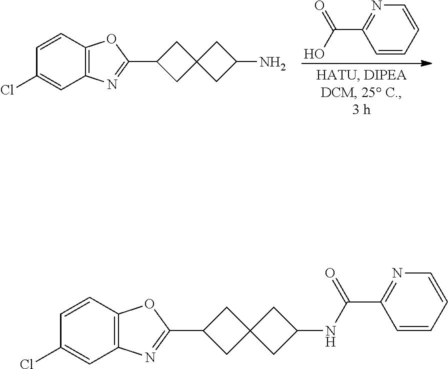 [SMILES: NC1CC2(C1)CC(c1nc3cc(Cl)ccc3o1)C2 ; O=C(O)c1ccccn1 ; O=C(NC1CC2(C1)CC(c1nc3cc(Cl)ccc3o1)C2)c1ccccn1]

To a solution of 6-(5-chloro-1,3-benzoxazol-2-yl)spiro[3.3]heptan-2-amine (Int-1, as the “AMINE” in Table 1) (100 mg, 0.38 mmol) and pyridine-2-carboxylic acid (70 mg, 0.57 mmol, as the “ACID” or “ACYL CHLORIDE” in Table 1) in DCM (4 mL) was added HATU (289 mg, 0.76 mmol) and DIPEA (0.2 mL, 1.14 mmol). Then the mixture was stirred at 25° C. for 3 h. The reaction mixture was diluted with DCM (40 ml). The solution was washed with water (10 ml×2), brine (10 ml), dried over Na₂SO₄ and concentrated in vacuum. The residue was purified by pre-HPLC to afford N-[6-(5-chloro-1,3-benzoxazol-2-yl)spiro[3.3]heptan-2-yl]pyridine-2-carboxamide (Example 1) (27 mg, 0.070 mmol) as a white solid.

MS obsd. (ESI⁺) [(M+H)⁺]: 368.1. ¹H NMR (DMSO-d₆, 400 MHz) δ: 8.92 (br d, J=8.2 Hz, 1H), 8.64 (d, J=4.6 Hz, 1H), 7.96-8.03 (m, 2H), 7.80 (d, J=2.0 Hz, 1H), 7.72 (d, J=8.7 Hz, 1H), 7.60 (t, J=6.0 Hz, 1H), 7.40 (dd, J=8.7, 1.6 Hz, 1H), 4.37 (sxt, J=8.2 Hz, 1H), 3.74 (quin, J=8.5 Hz, 1H), 2.54-2.69 (m, 3H), 2.44 (br d, J=8.4 Hz, 2H), 2.17-2.37 (m, 3H).

The following Example 2 to Example 58 were prepared in analogy to the procedure described for the preparation of Example 1, replacing 6-(5-chloro-1,3-benzoxazol-2-yl)spiro[3.3]heptan-2-amine (Int-1) with “AMINE”, and replacing pyridine-2-carboxylic acid with “ACID” or “ACYL CHLORIDE”. The “AMINE”, “ACID” and “ACYL CHLORIDE” are the reagents indicated in Table 1.

TABLE 1
Compounds synthesis and characterization

| | | AMINE, | |
|---|---|---|---|
| | | ACID or | |
| Exam- | | ACYL | |
| ples | Name and Structure | CHLORIDE | ¹H NMR and MS obsd. (ESI⁺) |
| 2 | N-[6-(5-chloro-1,3-benzoxazol- | AMINE: Int-1 | ¹H NMR (DMSO-d₆, 400 MHz) δ: |
| | 2-yl)spiro[3.3]heptan-2- | ACID: nicotinic | 8.96-9.03 (m, 1 H), 8.79 (br d, J = |
| | yl]pyridine-3-carboxamide | acid | 7.3 Hz, 1 H), 8.70 (dd, J = 4.7, 1.3 |
| | 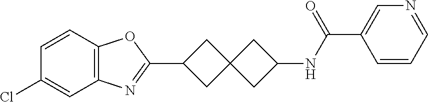 [SMILES: O=C(NC1CC2(C1)CC(c1nc3cc(Cl)ccc3o1)C2)c1cccnc1] | | Hz, 1 H), 8.17 (dt, J = 7.9, 1.8 Hz, 1 H), 7.80 (d, J = 2.0 Hz, 1 H), 7.72 (d, J = 8.7 Hz, 1 H), 7.50 (dd, J = 7.5, 4.8 Hz, 1 H), 7.40 (dd, J = 8.7, 2.1 Hz, 1 H), 4.34 (sxt, J = 8.0 Hz, 1 H), 3.75 (quin, J = 8.5 Hz, 1 H), 2.52-2.68 (m, 3 H), 2.46 (br d, J = |
| | | | 8.3 Hz, 2 H), 2.31-2.39 (m, 1 H), |
| | | | 2.08-2.28 (m, 2 H). |
| | | | MS obsd. (ESI⁺) [(M + H)⁺]: 368.1. |
| 3 | N-[6-(5-chloro-1,3-benzoxazol- | AMINE: Int-1 | ¹H NMR (CDCl₃, 400 MHz) δ: 8.74 |
| | 2-yl)spiro[3.3]heptan-2- | ACYL | (br d, J = 4.2 Hz, 2 H), 7.60-7.67 |
| | yl]pyridine-4-carboxamide | CHLORIDE: | (m, 3 H), 7.40 (d, J = 8.7 Hz, 1 H), |
| | 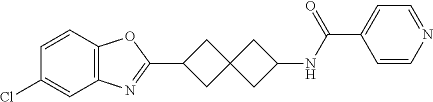 [SMILES: O=C(NC1CC2(C1)CC(c1nc3cc(Cl)ccc3o1)C2)c1ccncc1] | isonicotinic acid | 7.27-7.30 (m, 1 H), 6.38 (br d, J = 6.8 Hz, 1 H), 4.48-4.58 (m, 1 H), 3.63-3.76 (m, 1 H), 2.70-2.83 (m, 1 H), 2.56-2.67 (m, 4 H), 2.41- 2.53 (m, 1 H), 2.05-2.17 (m, 2 H). MS obsd. (ESI⁺) [(M + H)⁺]: 368.1. |
| 4 | N-[6-(5-chloro-1,3-benzoxazol- | AMINE: Int-1 | ¹H NMR (CDCl₃, 400 MHz) δ: 8.60 |
| | 2-yl)spiro[3.3]heptan-2-yl]-2- | ACID: 2- | (d, J = 5.1 Hz, 1 H), 7.64 (d, J = 2.0 |
| | methyl-pyridine-4- | methylpyridine- | Hz, 1 H), 7.49 (s, 1 H), 7.36-7.43 |
| | carboxamide | 4-carboxylic | (m, 2 H), 7.28 (d, J = 7.5 Hz, 1 H), |
| | 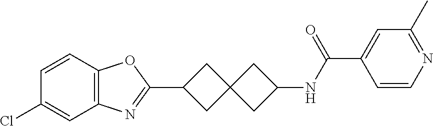 [SMILES: Cc1cc(C(=O)NC2CC3(C2)CC(c2nc4cc(Cl)ccc4o2)C3)ccn1] | acid | 6.35 (br d, J = 7.1 Hz, 1 H), 4.47- 4.57 (m, 1 H), 3.59-3.75 (m, 1 H), 2.72-2.79 (m, 1 H), 2.43-2.68 (m, 8 H), 2.01-2.17 (m, 2 H). MS obsd. (ESI+) [(M + H)+]: 382.1. |
| 5 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-5 | 8.65 (d, J = 7.5 Hz, 1 H), 8.04 (d, J = |
| | (oxetan-3-ylamino)pyridine-4- | | 5.3 Hz, 1 H), 7.80 (d, J = 2.0 Hz, |
| | carboxamide | | 1 H), 7.72 (d, J = 8.7 Hz, 1 H), 7.38- |
| | 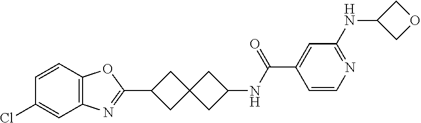 [SMILES: O=C(NC1CC2(C1)CC(c1nc3cc(Cl)ccc3o1)C2)c1ccnc(NC2COC2)c1] | | 7.48 (m, 2 H), 6.82-6.89 (m, 2 H), 4.77-4.92 (m, 3 H), 4.43 (t, J = 6.2 Hz, 2 H), 4.23-4.35 (m, 1 H), 3.69- 3.79 (m, 1 H), 2.53-2.68 (m, 3 H), 2.39-2.48 (m, 2 H), 2.26-2.38 (m, 1 H), 2.06-2.24 (m, 2 H). MS obsd. (ESI+) [(M + H)+]: 439.1. |
| 6 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-6 | 8.62 (d, J = 7.3 Hz, 1 H), 8.12 (d, J = |
| | (diethylamino)pyridine-4- | | 5.0 Hz, 1 H), 7.80 (d, J = 2.1 Hz, |
| | carboxamide | | 1 H), 7.72 (d, J = 8.7 Hz, 1 H), 7.40 |
| | 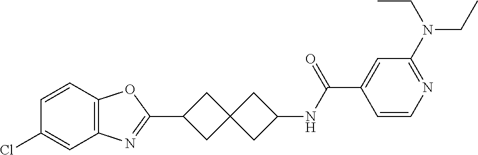 [SMILES: CCN(CC)c1cc(C(=O)NC2CC3(C2)CC(c2nc4cc(Cl)ccc4o2)C3)ccn1] | | (dd, J = 8.7, 2.2 Hz, 1 H), 6.85 (s, 1 H), 6.83 (dd, J = 5.1, 1.2 Hz, 1 H), 4.31 (sxt, J = 8.0 Hz, 1H), 3.75 (quin, J = 8.5 Hz, 1 H), 3.51 (q, J = 7.0 Hz, 4 H), 2.52-2.68 (m, 3 H), 2.40-2.48 (m, 2 H), 2.28-2.37 (m, 1), 2.08-2.26 (m, 2 H), 1.11 (t, J = 7.0 Hz, 6 H). MS obsd. (ESI+) [(M + H)+]: 439.2. |
| 7 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-7 | 8.64 (d, J = 7.3 Hz, 1 H), 8.14 (d, J = |
| | [ethyl(methyl)amino]pyridine- | | 5.0 Hz, 1 H), 7.80 (d, J = 2.2 Hz, |
| | 4-carboxamide | | 1 H), 7.72 (d, J = 8.7 Hz, 1 H), 7.40 |
| | 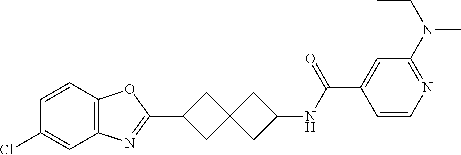 [SMILES: CCN(C)c1cc(C(=O)NC2CC3(C2)CC(c2nc4cc(Cl)ccc4o2)C3)ccn1] | | (dd, J = 8.7, 2.1 Hz, 1 H), 6.89 (s, 1 H), 6.87 (dd, J = 5.2, 1.2 Hz, 1 H), 4.31 (sxt, J = 8.1 Hz, 1 H), 3.75 (quin, J = 8.5 Hz, 1 H), 3.56-3.62 (m, 2 H), 3.00 (s, 3 H), 2.52-2.68 (m, 3 H), 2.29-2.48 (m, 3 H), 2.07- 2.26 (m, 2 H), 1.06 (t, J = 7.0 Hz, 3 H). MS obsd. (ESI+) [(M + H)+]: 425.2. |
| 8 | N-[6-(5-chloro-1,3-benzoxazol- | AMINE: Int-1 | ¹H NMR (CDCl₃, 400 MHz) δ: 8.19 |
| | 2-yl)spiro[3.3]heptan-2-yl]-2- | ACID: Int-8 | (d, J = 5.1 Hz, 1 H), 7.64 (d, J = 2.0 |
| | (1-piperidyl)pyridine-4- | | Hz, 1 H), 7.39 (d, J = 8.6 Hz, 1 H), |
| | carboxamide | | 7.28-7.30 (m, 1 H), 7.07 (br s, 1 |
| | 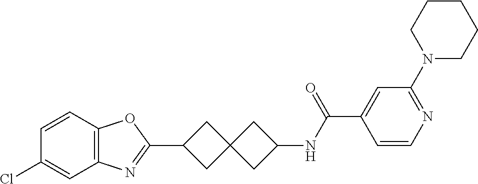 [SMILES: O=C(NC1CC2(C1)CC(c1nc3cc(Cl)ccc3o1)C2)c1ccnc(N2CCCCC2)c1] | | H), 6.70 (br s, 1 H), 6.33 (br s, 1 H), 4.45-4.55 (m, 1 H), 3.66-3.74 (m, 1 H), 3.53-3.66 (m, 4 H), 2.71- 2.78 (m, 1 H), 2.53-2.68 (m, 4 H), 2.38-2.50 (m, 1 H), 1.97-2.18 (m, 2 H), 1.44-1.84 (m, 6 H). MS obsd. (ESI+) [(M + H)+]: 451.1. |
| 9 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-9 | 8.69 (d, J = 7.3 Hz, 1 H), 8.21 (d, J = |
| | morpholino-pyridine-4- | | 5.1 Hz, 1 H), 7.80 (d, J = 2.1 Hz, |
| | carboxamide | | 1 H), 7.72 (d, J = 8.6 Hz, 1 H), 7.40 |
| | 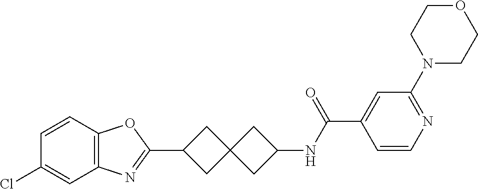 [SMILES: O=C(NC1CC2(C1)CC(c1nc3cc(Cl)ccc3o1)C2)c1ccnc(N2CCOCC2)c1] | | (dd, J = 8.6, 2.1 Hz, 1 H), 7.13 (s, 1 H), 7.02 (dd, J = 5.1, 1.2 Hz, 1 H), 4.32 (sxt, J = 8.1 Hz, 1 H), 3.64- 3.83 (m, 5 H), 3.43-3.57 (m, 4 H), 2.52-2.71 (m, 3 H), 2.30-2.48 (m, 3 H), 2.06-2.25 (m, 2 H). MS obsd. (ESI+) [(M + H)+]: 453.1. |
| 10 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-10 | 8.63 (d, J = 7.5 Hz, 1 H), 8.07 (d, J = |
| | (cyclopropylamino)pyridine-4- | | 5.2 Hz, 1 H), 7.80 (d, J = 2.0 Hz, |
| | carboxamide | | 1 H), 7.72 (d, J = 8.7 Hz, 1 H), 7.40 |
| | 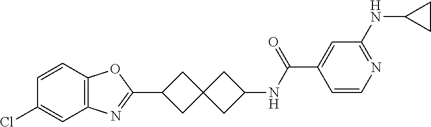 [SMILES: O=C(NC1CC2(C1)CC(c1nc3cc(Cl)ccc3o1)C2)c1ccnc(NC2CC2)c1] | | (dd, J = 8.7, 2.1 Hz, 1 H), 6.87- 6.96 (m, 3 H), 4.30 (sxt, J = 8.1 Hz, 1 H), 3.74 (quin, J = 8.5 Hz, 1 H), 2.52-2.68 (m, 3 H), 2.41-2.47 (m, 2 H), 2.27-2.36 (m, 1 H), 2.07- 2.25 (m, 2 H), 0.67-0.73 (m, 2 H), 0.39-0.45 (m, 2 H). MS obsd. (ESI+) [(M + H)+]: 423.1. |
| 11 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2- | 8.66 (d, J = 7.3 Hz, 1 H), 8.15 (d, J = |
| | (dimethylamino)pyridine-4- | (dimethylamino) | 5.1 Hz, 1 H), 7.80 (d, J = 2.1 Hz, |
| | carboxamide | pyridine-4- | 1 H), 7.72 (d, J = 8.7 Hz, 1 H), 7.40 |
| | 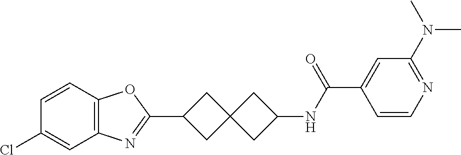 [SMILES: CN(C)c1cc(C(=O)NC2CC3(C2)CC(c2nc4cc(Cl)ccc4o2)C3)ccn1] | carboxylic acid | (dd, J = 8.7, 2.2 Hz, 1 H), 6.94 (s, 1 H), 6.90 (dd, J = 5.1, 1.1 Hz, 1 H), 4.32 (sxt, J = 8.1 Hz, 1 H), 3.75 (quin, J = 8.5 Hz, 1 H), 3.06 (s, 6 H), 2.53-2.68 (m, 3 H), 2.30-2.48 (m, 3 H), 2.08-2.25 (m, 2 H). MS obsd. (ESI+) [(M + H)+]: 411.1. |
| 12 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-11 | 8.64 (d, J = 7.5 Hz, 1 H), 8.14 (d, J = |
| | [isopropyl(methyl)amino]pyri- | | 5.1 Hz, 1 H), 7.80 (d, J = 2.1 Hz, |
| | dine-4-carboxamide | | 1 H), 7.72 (d, J = 8.7 Hz, 1 H), 7.40 |
| | 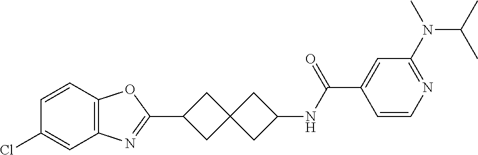 [SMILES: CC(C)N(C)c1cc(C(=O)NC2CC3(C2)CC(c2nc4cc(Cl)ccc4o2)C3)ccn1] | | (dd, J = 8.6, 2.1 Hz, 1 H), 6.90 (s, 1 H), 6.87 (dd, J = 5.1, 1.1 Hz, 1 H), 4.86 (spt, J = 6.7 Hz, 1H), 4.31 (sxt, J = 8.1 Hz, 1 H), 3.75 (quin, J = 8.5 Hz, 1 H), 2.83 (s, 3 H), 2.53-2.68 (m, 3 H), 2.41-2.48 (m, 2 H), 2.29- 2.37 (m, 1 H), 2.08-2.24 (m, 2 H), 1.12 (d, J = 6.7 Hz, 6 H). MS obsd. (ESI+) [(M + H)+]: 439.2. |
| 13 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-12 | 8.86 (d, J = 7.2 Hz, 1 H), 8.61 (s, 1 |
| | (2-oxopyrrolidin-1-yl)pyridine- | | H), 8.48 (d, J = 5.2 Hz, 1 H), 7.80 |
| | 4-carboxamide | | (d, J = 2.1 Hz, 1 H), 7.72 (d, J = 8.7 |
| | 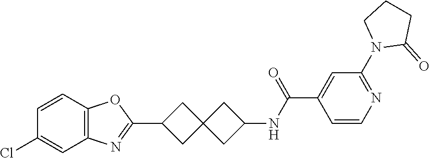 [SMILES: O=C(NC1CC2(C1)CC(c1nc3cc(Cl)ccc3o1)C2)c1ccnc(N2CCCC2=O)c1] | | Hz, 1 H), 7.47 (dd, J = 5.2, 1.5 Hz, 1 H), 7.40 (dd, J = 8.7, 2.2 Hz, 1 H), 4.29-4.37 (m, 1 H), 4.00 (t, J = 7.1 Hz, 2 H), 3.75 (quin, J = 8.5 Hz, 1 H), 2.52-2.64 (m, 5 H), 2.42-2.48 (m, 2 H), 2.31-2.37 (m, 1 H), 2.18- 2.25 (m, 1 H), 2.02-2.16 (m, 3 H). MS obsd. (ESI+) [(M + H)+]: 451.1. |
| 14 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-13 | 9.13 (br d, J = 7.2 Hz, 1 H), 8.74 (d, |
| | (4-methylthiazol-2-yl)pyridine- | | J = 5.0 Hz, 1 H), 8.46 (s, 1 H), 7.75- |
| | 4-carboxamide | | 7.86 (m, 2 H), 7.71 (d, J = 8.7 Hz, |
| | 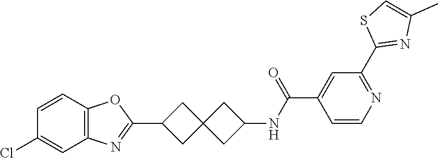 [SMILES: Cc1csc(-c2cc(C(=O)NC3CC4(C3)CC(c3nc5cc(Cl)ccc5o3)C4)ccn2)n1] | | 1 H), 7.46 (s, 1 H), 7.32-7.44 (m, 1 H), 4.38 (sxt, J = 7.9 Hz, 1 H), 3.75 (quin, J = 8.4 Hz, 1 H), 2.53-2.77 (m, 3 H), 2.48 (s, 3 H), 2.41-2.47 (m, 2 H), 2.32-2.40 (m, 1 H), 2.15- 2.31 (m, 2 H). MS obsd. (ESI+) [(M + H)+]: 465.1. |
| 15 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-14 | 9.10 (d, J = 7.3 Hz, 1 H), 8.73 (d, J = |
| | (5-methylthiazol-2-yl)pyridine- | | 5.0 Hz, 1 H), 8.47 (s, 1 H), 7.76- |
| | 4-carboxamide | | 7.85 (m, 2 H), 7.70-7.76 (m, 2 H), |
| | 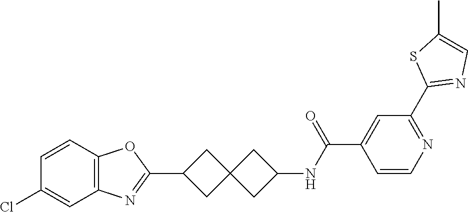 [SMILES: Cc1cnc(-c2cc(C(=O)NC3CC4(C3)CC(c3nc5cc(Cl)ccc5o3)C4)ccn2)s1] | | 7.40 (dd, J = 8.6, 2.1 Hz, 1 H), 4.31- 4.42 (m, 1 H), 3.76 (quin, J = 8.4 Hz, 1 H), 2.52-2.66 (m, 6 H), 2.43- 2.48 (m, 2 H), 2.31-2.42 (m, 1 H), 2.07-2.29 (m, 2 H). MS obsd. (ESI+) [(M + H)+]: 465.1. |
| 16 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-15 | 9.13 (br d, J = 6.7 Hz, 1 H), 8.76 (br |
| | thiazol-2-yl-pyridine-4- | | d, J = 4.5 Hz, 1 H), 8.54 (br s, 1 H), |
| | carboxamide | | 8.05 (br s, 1 H), 7.92 (br s, 1 H), |
| | 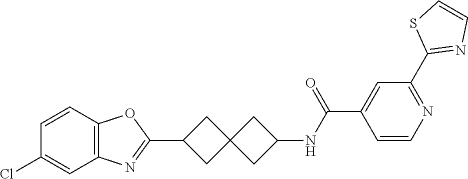 [SMILES: O=C(NC1CC2(C1)CC(c1nc3cc(Cl)ccc3o1)C2)c1ccnc(-c2nccs2)c1] | | 7.81-7.88 (m, 1 H), 7.79 (br s, 1 H), 7.70 (br d, J = 8.6 Hz, 1 H), 7.39 (br d, J = 8.3 Hz, 1 H), 4.28-4.48 (m, 1 H), 3.74 (br t, J = 8.3 Hz, 1 H), 2.49-2.68 (m, 4 H), 2.41-2.46 (m, 1 H), 2.36 (br s, 1 H), 2.12- 2.32 (m, 2 H). MS obsd. (ESI+) [(M + H)+]: 451.1. |
| 17 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2- | 8.81 (d, J = 7.3 Hz, 1 H), 8.28 (d, J = |
| | methoxy-pyridine-4- | methoxypyridine- | 5.3 Hz, 1 H), 7.80 (d, J = 2.1 Hz, |
| | carboxamide | 4-carboxylic | 1 H), 7.72 (d, J = 8.6 Hz, 1 H), 7.40 |
| | 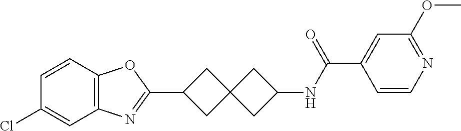 [SMILES: COc1cc(C(=O)NC2CC3(C2)CC(c2nc4cc(Cl)ccc4o2)C3)ccn1] | acid | (dd, J = 8.6, 2.1 Hz, 1 H), 7.33 (dd, J = 5.3, 1.3 Hz, 1 H), 7.18 (s, 1 H), 4.31 (sxt, J = 8.0 Hz, 1 H), 3.89 (s, 3 H), 3.74 (quin, J = 8.5 Hz, 1 H), 2.52-2.68 (m, 3 H), 2.30-2.47 (m, 3 H), 2.08-2.24 (m, 2 H). MS obsd. (ESI+) [(M + H)+]: 398.1. |
| 18 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2- | 9.05 (d, J = 7.2 Hz, 1 H), 8.90 (dd, J = |
| | cyano-pyridine-4- | cyanopyridine- | 5.1, 0.8 Hz, 1 H), 8.37 (dd, J = |
| | carboxamide | 4-carboxylic | 1.6, 0.9 Hz, 1 H), 8.06 (dd, J = 5.0, |
| | 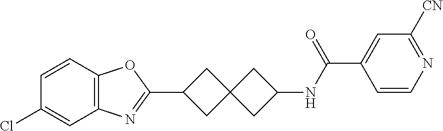 [SMILES: N#Cc1cc(C(=O)NC2CC3(C2)CC(c2nc4cc(Cl)ccc4o2)C3)ccn1] | acid | 1.7 Hz, 1 H), 7.80 (d, J = 1.8 Hz, 1 H), 7.72 (d, J = 8.7 Hz, 1 H), 7.40 (dd, J = 8.7, 2.2 Hz, 1 H), 4.28- 4.38 (m, 1 H), 3.75 (quin, J = 8.5 Hz, 1 H), 2.52-2.68 (m, 3 H), 2.44- 2.48 (m, 2 H), 2.32-2.40 (m, 1 H), 2.07-2.26 (m, 2 H). MS obsd. (ESI+) [(M + H)+]: 393.0. |
| 19 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2- | 9.17 (d, J = 7.3 Hz, 1 H), 8.86 (d, J = |
| | sulfamoyl-pyridine-4- | sulfamoylpyridine- | 4.9 Hz, 1 H), 8.30-8.34 (m, 1 H), |
| | carboxamide | 4-carboxylic | 8.00 (dd, J = 5.0, 1.5 Hz, 1 H), 7.78- |
| | 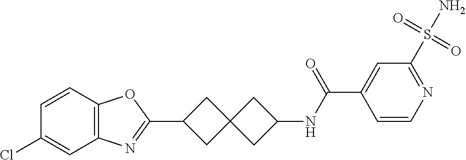 [SMILES: NS(=O)(=O)c1cc(C(=O)NC2CC3(C2)CC(c2nc4cc(Cl)ccc4o2)C3)ccn1] | acid | 7.83 (m, 1 H), 7.68-7.75 (m, 1 H), 7.56 (br s, 2 H), 7.36-7.44 (m, 1 H), 4.30-4.43 (m, 1 H), 3.69-3.81 (m, 1 H), 2.54-2.68 (m, 3 H), 2.33- 2.48 (m, 3 H), 2.11-2.30 (m, 2 H). MS obsd. (ESI+) [(M + H)+]: 447.1. |
| 20 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-16 | 8.77 (d, J = 7.3 Hz, 1 H), 8.24 (d, J = |
| | isopropoxy-pyridine-4- | | 5.3 Hz, 1 H), 7.80 (d, J = 1.8 Hz, |
| | carboxamide | | 1 H), 7.72 (d, J = 8.9 Hz, 1 H), 7.40 |
| | 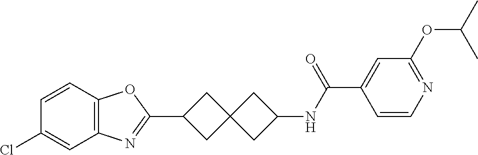 [SMILES: CC(C)Oc1cc(C(=O)NC2CC3(C2)CC(c2nc4cc(Cl)ccc4o2)C3)ccn1] | | (dd, J = 8.7, 2.2 Hz, 1 H), 7.27 (dd, J = 5.3, 1.4 Hz, 1 H), 7.11 (s, 1 H), 5.26 (spt, J = 6.2 Hz, 1 H), 4.30 (sxt, J = 8.0 Hz, 1 H), 3.74 (quin, J = 8.5 Hz, 1 H), 2.52-2.68 (m, 3 H), 2.41- 2.47 (m, 2 H), 2.28-2.36 (m, 1 H), 2.04-2.25 (m, 2 H), 1.30 (d, J = 6.2 Hz, 6 H). MS obsd. (ESI+) [(M + H)+]: 426.2. |
| 21 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2- | 8.86 (d, J = 4.9 Hz, 1 H), 7.98 (s, 1 |
| | (trifluoromethyl)pyridine-4- | (trifluoromethyl) | H), 7.80 (dd, J = 5.0, 1.3 Hz, 1 H), |
| | carboxamide | isonicotinic | 7.65 (d, J = 1.7 Hz, 1 H), 7.40 (d, J = |
| | 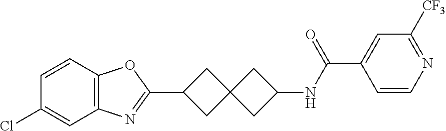 [SMILES: O=C(NC1CC2(C1)CC(c1nc3cc(Cl)ccc3o1)C2)c1ccnc(C(F)(F)F)c1] | acid | 8.6 Hz, 1 H), 7.27-7.30 (m, 1 H), 6.33 (br d, J = 7.2 Hz, 1 H), 4.49- 4.60 (m, 1 H), 3.72 (quin, J = 8.5 Hz, 1 H), 2.74-2.82 (m, 1 H), 2.58- 2.68 (m, 4 H), 2.44-2.53 (m, 1 H), 2.04-2.20 (m, 2 H). MS obsd. (ESI+) [(M + H)+]: 436.1. |
| 22 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-17 | 10.52 (br s, 1 H), 8.80 (d, J = 7.3 |
| | (methylsulfamoylamino)pyri- | | Hz, 1 H), 8.33 (br d, J = 4.8 Hz, 1 |
| | dine-4-carboxamide | | H), 7.80 (d, J = 2.1 Hz, 1 H), 7.72 |
| | 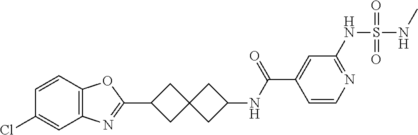 [SMILES: CNS(=O)(=O)Nc1cc(C(=O)NC2CC3(C2)CC(c2nc4cc(Cl)ccc4o2)C3)ccn1] | | (d, J = 8.7 Hz, 1 H), 7.40 (dd, J = 8.7, 2.1 Hz, 1 H), 7.28-7.35 (m, 2 H), 7.24 (br s, 1 H), 4.25-4.35 (m, 1 H), 3.68-3.80 (m, 1 H), 2.55- 2.68 (m, 3 H), 2.43-2.47 (m, 5 H), 2.30-2.36 (m, 1 H), 2.07-2.24 (m, 2 H). MS obsd. (ESI+) [(M + H)+]: 476.0. |
| 23 | N-[6-(5-chloro-1,3-benzoxazol- | AMINE: Int-1 | ¹H NMR (MeOH-d₄, 400 MHz) δ: |
| | 2-yl)spiro[3.3]heptan-2-yl]-2- | ACID: 2- | 8.87 (dd, J = 4.9, 0.7 Hz, 1 H), 8.43 |
| | methylsulfonyl-pyridine-4- | methylsulfonyl- | (dd, J = 1.6, 0.7 Hz, 1 H), 8.03 (dd, |
| | carboxamide | pyridine-4- | J = 5.0, 1.7 Hz, 1 H), 7.64 (d, J = |
| | | carboxylic acid | 1.8 Hz, 1 H), 7.56 (d, J = 8.7 Hz, 1 |
| | 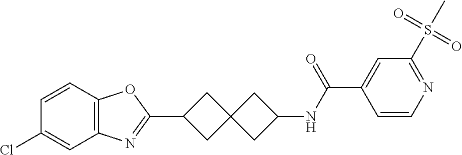 [SMILES: CS(=O)(=O)c1cc(C(=O)NC2CC3(C2)CC(c2nc4cc(Cl)ccc4o2)C3)ccn1] | | H), 7.36 (dd, J = 8.7, 2.1 Hz, 1 H), 4.44 (quin, J = 8.2 Hz, 1 H), 3.76 (quin, J = 8.5 Hz, 1 H), 3.25-3.28 (m, 3 H), 2.45-2.73 (m, 6 H), 2.17- 2.31 (m, 2 H). MS obsd. (ESI+) [(M + H)+]: 446.1. |
| 24 | N-[6-(5-chloro-1,3-benzoxazol- | AMINE: Int-1 | ¹H NMR (MeOH-d₄, 400 MHz) δ: |
| | 2-yl)spiro[3.3]heptan-2-yl]-5- | ACID: Int-18 | 9.25 (d, J = 2.0 Hz, 1 H), 9.21 (d, J = |
| | methylsulfonyl-pyridine-3- | | 2.1 Hz, 1 H), 8.72 (t, J = 2.1 Hz, 1 |
| | carboxamide | | H), 7.64 (d, J = 1.7 Hz, 1 H), 7.56 |
| | 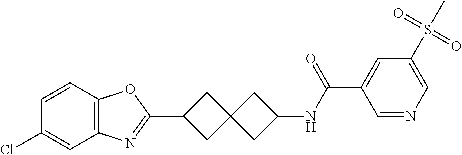 [SMILES: CS(=O)(=O)c1cncc(C(=O)NC2CC3(C2)CC(c2nc4cc(Cl)ccc4o2)C3)c1] | | (d, J = 8.7 Hz, 1 H), 7.36 (dd, J = 8.7, 2.1 Hz, 1 H), 4.45 (t, J = 8.3 Hz, 1 H), 3.76 (t, J = 8.4 Hz, 1 H), 3.25 (s, 3 H), 2.62-2.74 (m, 3 H), 2.43- 2.58 (m, 3 H), 2.16-2.31 (m, 2 H). MS obsd. (ESI+) [(M + H)+]: 446.1. |
| 25 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-19 | 9.23 (d, J = 7.2 Hz, 1 H), 8.91 (d, J = |
| | (oxetan-3-ylsulfonyl)pyridine- | | 5.4 Hz, 1 H), 8.47 (s, 1 H), 8.11 |
| | 4-carboxamide | | (dd, J = 4.9, 1.6 Hz, 1 H), 7.80 (d, J = |
| | 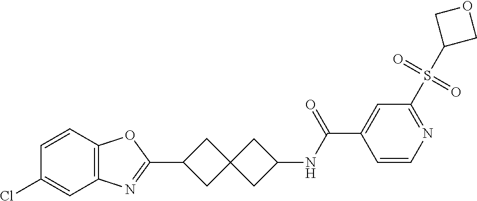 [SMILES: O=C(NC1CC2(C1)CC(c1nc3cc(Cl)ccc3o1)C2)c1ccnc(S(=O)(=O)C2COC2)c1] | | 2.1 Hz, 1 H), 7.72 (d, J = 8.7 Hz, 1 H), 7.40 (dd, J = 8.6, 2.1 Hz, 1 H), 5.07-5.14 (m, 1 H), 4.86 (d, J = 7.1 Hz, 4 H), 4.35 (sxt, J = 8.0 Hz, 1 H), 3.76 (quin, J = 8.5 Hz, 1 H), 2.52- 2.68 (m, 3 H), 2.32-2.48 (m, 3 H), 2.12-2.27 (m, 2 H). MS obsd. (ESI+) [(M + H)+]: 488.0. |
| 26 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: Int-20 | 9.23 (d, J = 7.2 Hz, 1 H), 8.95 (d, J = |
| | ethylsulfonyl-pyridine-4- | | 5.1 Hz, 1 H), 8.44 (d, J = 0.9 Hz, |
| | carboxamide | | 1 H), 8.11 (dd, J = 5.0, 1.7 Hz, 1 H), |
| | 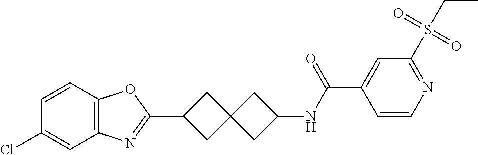 [SMILES: CCS(=O)(=O)c1cc(C(=O)NC2CC3(C2)CC(c2nc4cc(Cl)ccc4o2)C3)ccn1] | | 7.81 (d, J = 2.0 Hz, 1 H), 7.72 (d, J = 8.7 Hz, 1 H), 7.40 (dd, J = 8.6, 2.1 Hz, 1 H), 4.36 (sxt, J = 8.0 Hz, 1 H), 3.76 (quin, J = 8.5 Hz, 1 H), 3.49 (q, J = 7.3 Hz, 2 H), 2.52-2.71 (m, 3 H), 2.42-2.49 (m, 2 H), 2.31-2.41 (m, 1 H), 2.13-2.29 (m, 2 H), 1.14 (t, J = 7.4 Hz, 3 H). MS obsd. (ESI+) [(M + H)+]: 460.1. |
| 27 | N-[6-(5-chloro-1,3-benzoxazol- | AMINE: Int-1 | ¹H NMR (CDCl₃, 400 MHz) δ: 7.64 |
| | 2-yl)spiro[3.3]heptan-2-yl]-2,2- | ACYL | (d, J = 1.71 Hz, 1 H), 7.39 (d, J = |
| | dimethyl-propanamide | CHLORIDE: | 8.56 Hz, 1 H), 7.20-7.32 (m, 1 H), |
| | 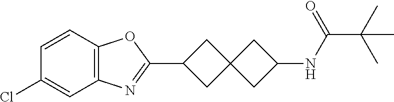 [SMILES: CC(C)(C)C(=O)NC1CC2(C1)CC(c1nc3cc(Cl)ccc3o1)C2] | 2,2- dimethylpropanoyl chloride | 5.66 (br d, J = 6.60 Hz, 1 H), 4.29 (sxt, J = 7.95 Hz, 1 H), 3.67 (quin, J = 8.56 Hz, 1 H), 2.37-2.71 (m, 6 H), 1.85-1.99 (m, 2 H), 1.18 (s, 9 H). MS obsd. (ESI+) [(M + H)+]: 347.1. |
| 28 | N-[6-(5-chloro-1,3-benzoxazol- | AMINE: Int-1 | ¹H NMR (CDCl₃, 400 MHz) δ: 7.64 |
| | 2-yl)spiro[3.3]heptan-2-yl]-3- | ACID: 3- | (d, J = 2.08 Hz, 1 H), 7.39 (d, J = |
| | methyl-butanamide | methylbutanoic | 8.56 Hz, 1 H), 7.25-7.29 (m, 1 H), |
| | 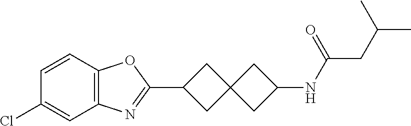 [SMILES: CC(C)CC(=O)NC1CC2(C1)CC(c1nc3cc(Cl)ccc3o1)C2] | acid | 5.55 (br d, J = 6.48 Hz, 1 H), 4.34 (sxt, J = 8.02 Hz, 1 H), 3.67 (quin, J = 8.59 Hz, 1 H), 2.62-2.71 (m, 1 H), 2.37-2.62 (m, 5 H), 2.04-2.15 (m, 1 H), 2.01 (s, 1 H), 1.99 (s, 1 H), 1.86-1.98 (m, 2 H), 0.95 (dd, J = 0.67, 6.54 Hz, 6 H). MS obsd. (ESI+) [(M + H)+]: 347.2. |
| 29 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-3- | ACID: 3- | 8.03 (d, J = 7.46 Hz, 1 H), 7.79 (d, J = |
| | (trifluoromethyl)cyclobutanecar- | (trifluoromethyl) | 1.96 Hz, 1 H), 7.71 (d, J = 8.56 |
| | boxamide | cyclobutanecar- | Hz, 1 H), 7.40 (dd, J = 2.20, 8.68 |
| | 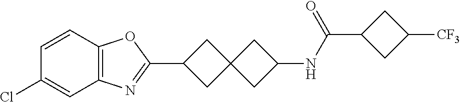 [SMILES: O=C(NC1CC2(C1)CC(c1nc3cc(Cl)ccc3o1)C2)C1CC(C(F)(F)F)C1] | boxylic acid | Hz, 1 H), 4.07 (sxt, J = 8.00 Hz, 1 H), 3.71 (quin, J = 8.47 Hz, 1 H), 3.10 (qd, J = 9.16, 18.23 Hz, 1 H), 2.89 (quin, J = 8.86 Hz, 1 H), 2.43- 2.60 (m, 3 H), 2.36-2.42 (m, 2 H), 2.21-2.28 (m, 1 H), 2.16 (t, J = 8.68 Hz, 4 H), 1.94-2.03 (m, 1 H), |
| | | | 1.89 (dd, J = 8.74, 11.19 Hz, 1 H). |
| | | | MS obsd. (ESI+) [(M + H)+]: 412.9. |
| 30 | N-[6-(5-chloro-1,3-benzoxazol- | AMINE: Int-1 | ¹H NMR (CDCl₃, 400 MHz) δ: 7.64 |
| | 2-yl)spiro[3.3]heptan-2-yl]-1- | ACID: 1- | (d, J = 1.96 Hz, 1 H), 7.39 (d, J = |
| | methyl-5-oxo-pyrrolidine-3- | methyl-5-oxo- | 8.68 Hz, 1 H), 7.27 (br dd, J = 2.08, |
| | carboxamide | pyrrolidine-3- | 8.56 Hz, 1 H), 5.86 (br d, J = 7.21 |
| | 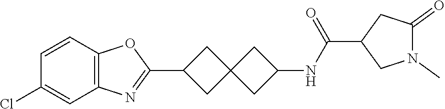 [SMILES: CN1CC(C(=O)NC2CC3(C2)CC(c2nc4cc(Cl)ccc4o2)C3)CC1=O] | carboxylic acid | Hz, 1 H), 4.33 (sxt, J = 8.04 Hz, 1 H), 3.61-3.70 (m, 2 H), 3.49 (dt, J = 2.57, 9.29 Hz, 1 H), 3.00 (quin, J = 8.31 Hz, 1 H), 2.85 (s, 3 H), 2.38- 2.71 (m, 8 H), 1.90-2.04 (m, 2 H). MS obsd. (ESI+) [(M + H)+]: 388.1. |
| 31 | 1-tert-butyl-N-[6-(5-chloro-1,3- | AMINE: Int-1 | ¹H NMR (CDCl₃, 400 MHz) δ: 7.65 |
| | benzoxazol-2- | ACID: Int-21 | (s, 1 H), 7.40 (d, J = 8.56 Hz, 1 H), |
| | yl)spiro[3.3]heptan-2-yl]-5- | | 7.29 (br d, J = 1.83 Hz, 1 H), 5.81 |
| | oxo-pyrrolidine-3- | | (br s, 1 H), 4.34 (sxt, J = 7.75 Hz, 1 |
| | carboxamide | | H), 3.56-3.77 (m, 3 H), 2.92 (br t, J = |
| | 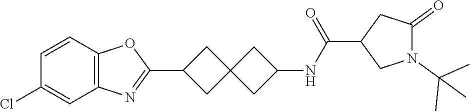 [SMILES: CC(C)(C)N1CC(C(=O)NC2CC3(C2)CC(c2nc4cc(Cl)ccc4o2)C3)CC1=O] | | 8.44 Hz, 1 H), 2.38-2.75 (m, 8 H), 1.91-2.07 (m, 2 H), 1.42 (s, 9 H). MS obsd. (ESI+) [(M + H)+]: 430.2. |
| 32 | N-[6-(5-chloro-1,3-benzoxazol- | AMINE: Int-1 | ¹H NMR (CDCl₃, 400 MHz) δ: 7.65 |
| | 2-yl)spiro[3.3]heptan-2-yl]-5- | ACID: Int-22 | (d, J = 1.96 Hz, 1 H), 7.58 (d, J = |
| | oxo-1-phenyl-pyrrolidine-3- | | 8.56 Hz, 2 H), 7.34-7.43 (m, 3 H), |
| | carboxamide | | 7.29 (d, J = 2.08 Hz, 1 H), 7.12- |
| | 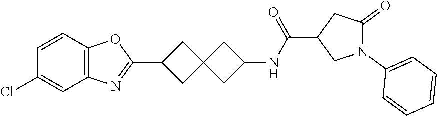 [SMILES: O=C(NC1CC2(C1)CC(c1nc3cc(Cl)ccc3o1)C2)C1CC(=O)N(c2ccccc2)C1] | | 7.21 (m, 1 H), 5.72 (br d, J = 7.34 Hz, 1 H), 4.37 (sxt, J = 8.05 Hz, 1 H), 4.13-4.21 (m, 1 H), 3.93 (dt, J = 2.69, 9.11 Hz, 1 H), 3.69 (quin, J = 8.56 Hz, 1 H), 3.10 (quin, J = 8.65 Hz, 1 H), 2.89-3.00 (m, 1 H), 2.74- 2.84 (m, 1 H), 2.65-2.73 (m, 1 H), 2.61 (d, J = 7.82 Hz, 2 H), 2.39- |
| | | | 2.58 (m, 3 H), 1.91-2.09 (m, 2 H). |
| | | | MS obsd. (ESI+) [(M + H)+]: 450.1. |
| 33 | N-[6-(5-chloro-1,3-benzoxazol- | AMINE: Int-1 | ¹H NMR (CDCl₃, 400 MHz) δ: 7.64 |
| | 2-yl)spiro[3.3]heptan-2-yl]-1- | ACID: Int-23 | (d, J = 1.71 Hz, 1 H), 7.33-7.42 (m, |
| | (3-chloro-2-methyl-phenyl)-5- | | 2 H), 7.28 (d, J = 1.10 Hz, 1 H), |
| | oxo-pyrrolidine-3- | | 7.14-7.20 (m, 1 H), 7.07-7.12 (m, |
| | carboxamide | | 1 H), 5.75 (br d, J = 7.34 Hz, 1 H), |
| | 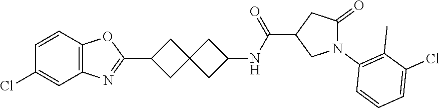 [SMILES: Cc1c(Cl)cccc1N1CC(C(=O)NC2CC3(C2)CC(c2nc4cc(Cl)ccc4o2)C3)CC1=O] | | 4.36 (sxt, J = 8.07 Hz, 1 H), 3.97 (ddd, J = 1.22, 6.54, 9.72 Hz, 1 H), 3.74-3.83 (m, 1 H), 3.68 (dquin, J = 2.63, 8.54 Hz, 1 H), 3.15 (quin, J = 7.92 Hz, 1 H), 2.73-2.92 (m, 2 H), 2.68 (dt, J = 7.03, 11.83 Hz, 1 H), 2.61 (d, J = 8.68 Hz, 2 H), 2.38- |
| | | | 2.57 (m, 3 H), 2.27 (s, 3 H), 1.89- |
| | | | 2.06 (m, 2 H). |
| | | | MS obsd. (ESI+) [(M + H)+]: 498.1. |
| 34 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-1- | ACID: 1- | 8.10 (br d, J = 7.34 Hz, 1 H), 7.78 |
| | methyl-2-oxo-piperidine-4- | methyl-2-oxo- | (d, J = 1.47 Hz, 1 H), 7.70 (d, J = |
| | carboxamide | piperidine-4- | 8.68 Hz, 1 H), 7.39 (dd, J = 1.77, |
| | 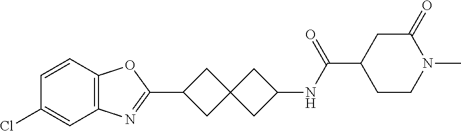 [SMILES: CN1CCC(C(=O)NC2CC3(C2)CC(c2nc4cc(Cl)ccc4o2)C3)CC1=O] | carboxylic acid | 8.62 Hz, 1 H), 4.07 (qd, J = 7.86, 15.67 Hz, 1 H), 3.70 (quin, J = 8.38 Hz, 1 H), 3.22 (br s, 2 H), 2.78 (s, 3 H), 2.44-2.62 (m, 4 H), 2.40 (br d, J = 8.19 Hz, 2 H), 2.20-2.32 (m, 3 H), 1.95-2.06 (m, 1 H), 1.84-1.94 (m, 2 H), 1.63-1.78 (m, 1 H). MS obsd. (ESI+) [(M + H)+]: 402.0. |
| 35 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2-(1- | 8.09 (d, J = 7.3 Hz, 1 H), 7.79 (d, J = |
| | (1-methyl-5-oxo-pyrrolidin-3- | methyl-5-oxo- | 2.0 Hz, 1 H), 7.71 (d, J = 8.6 Hz, |
| | yl)acetamide | pyrrolidin-3- | 1 H), 7.39 (dd, J = 8.7, 2.2 Hz, 1 H), |
| | 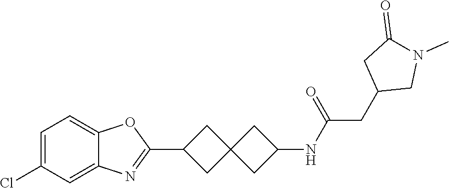 [SMILES: CN1CC(CC(=O)NC2CC3(C2)CC(c2nc4cc(Cl)ccc4o2)C3)CC1=O] | yl)acetic acid | 4.07 (sxt, J = 7.9 Hz, 1 H), 3.71 (quin, J = 8.5 Hz, 1 H), 3.39-3.46 (m, 1 H), 2.98 (ddd, J = 9.7, 5.8, 1.7 Hz, 1 H), 2.68 (s, 3 H), 2.52-2.62 (m, 2 H), 2.22-2.48 (m, 6 H), 2.17 (d, J = 7.3 Hz, 2 H), 1.82-2.04 (m, 3 H). MS obsd. (ESI+) [(M + H)+]: 402.4. |
| 36 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2-(1- | 8.20 (d, J = 7.3 Hz, 1 H), 7.79 (d, J = |
| | (1-methyl-5-oxo-pyrrolidin-2- | methyl-5-oxo- | 2.1 Hz, 1 H), 7.71 (d, J = 8.7 Hz, |
| | yl)acetamide | pyrrolidin-2- | 1 H), 7.40 (dd, J = 8.7, 2.2 Hz, 1 H), |
| | 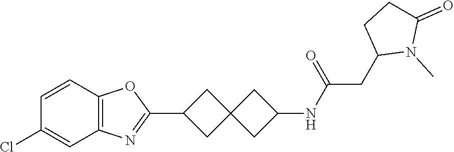 [SMILES: CN1C(=O)CCC1CC(=O)NC1CC2(C1)CC(c1nc3cc(Cl)ccc3o1)C2] | yl)acetic acid | 4.03-4.14 (m, 1 H), 3.67-3.80 (m, 2 H), 2.62-2.67 (m, 3 H), 2.52- 2.60 (m, 2 H), 2.39-2.48 (m, 4 H), 2.19-2.30 (m, 2 H), 1.95-2.16 (m, 4 H), 1.89 (dd, J = 11.0, 8.8 Hz, 1 H), 1.59-1.73 (m, 1 H). MS obsd. (ESI+) [(M + H)+]: 402.2. |
| 37 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2-(5- | 8.11 (br d, J = 7.3 Hz, 1 H), 7.76- |
| | (5-oxopyrrolidin-2- | oxopyrrolidin- | 7.82 (m, 1 H), 7.71 (d, J = 8.6 Hz, 1 |
| | yl)acetamide | 2-yl)acetic acid | H), 7.51 (s. 1 H), 7.39 (dd, J = 8.6, |
| | 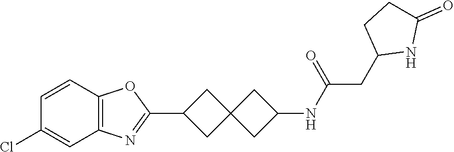 [SMILES: O=C1CCC(CC(=O)NC2CC3(C2)CC(c2nc4cc(Cl)ccc4o2)C3)N1] | | 1.7 Hz, 1 H), 4.08 (sxt, J = 7.8 Hz, 1 H), 3.65-3.84 (m, 2 H), 2.51-2.60 (m, 2 H), 2.37-2.47 (m, 3 H), 2.22- 2.32 (m, 2 H), 2.05-2.17 (m, 4 H), 1.85-2.03 (m, 2 H), 1.54-1.74 (m, 1 H). MS obsd. (ESI+) [(M + H)+]: 388.0. |
| 38 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-3- | ACID: 3- | 8.24 (d, J = 7.34 Hz, 1 H), 7.80 (d, J = |
| | sulfamoyl-propanamide | sulfamoylpro- | 1.96 Hz, 1 H), 7.71 (d, J = 8.56 |
| | 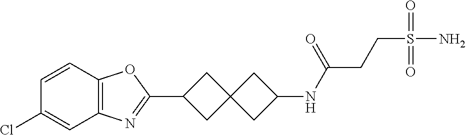 [SMILES: NS(=O)(=O)CCC(=O)NC1CC2(C1)CC(c1nc3cc(Cl)ccc3o1)C2] | panoic acid | Hz, 1 H), 7.40 (dd, J = 2.20, 8.68 Hz, 1 H), 6.84 (s, 2 H), 4.08 (sxt, J = 7.97 Hz, 1 H), 3.71 (quin, J = 8.47 Hz, 1 H), 3.10-3.21 (m, 2 H), 2.53- 2.61 (m, 2 H), 2.37-2.48 (m, 5 H), 2.21-2.30 (m, 1 H), 1.99 (dd, J = 8.93, 10.76 Hz, 1 H), 1.90 (dd, J = 8.80, 11.25 Hz, 1 H). MS obsd. (ESI+) [(M + H)+]: 398.1. |
| 39 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-4- | ACID: 4- | 8.07 (d, J = 7.46 Hz, 1 H), 7.79 (d, J = |
| | sulfamoyl-butanamide | sulfamoyl- | 2.08 Hz, 1 H), 7.71 (d, J = 8.68 |
| | 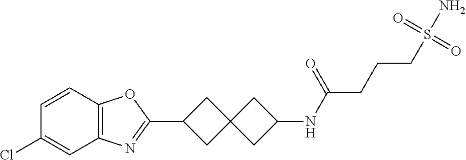 [SMILES: NS(=O)(=O)CCCC(=O)NC1CC2(C1)CC(c1nc3cc(Cl)ccc3o1)C2] | butanoic acid | Hz, 1 H), 7.40 (dd, J = 2.14, 8.62 Hz, 1 H), 6.77 (s, 2 H), 4.08 (qd, J = 8.03, 15.89 Hz, 1 H), 3.71 (quin, J = 8.47 Hz, 1 H), 2.89-2.99 (m, 2 H), 2.36-2.60 (m, 5 H), 2.21-2.29 (m, 1 H), 2.17 (t, J = 7.34 Hz, 2 H), 1.98 (br dd, J = 8.93, 10.51 Hz, 1 H), 1.83-1.93 (m, 3 H). MS obsd. (ESI+) [(M + H)+]: 412.1. |
| 40 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-1,1- | ACID: 1,1- | 8.34 (d, J = 7.21 Hz, 1 H), 7.79 (d, J = |
| | dioxo-thiolane-3-carboxamide | dioxothiolane- | 1.96 Hz, 1 H), 7.71 (d, J = 8.68 |
| | 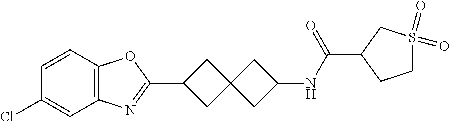 [SMILES: O=C(NC1CC2(C1)CC(c1nc3cc(Cl)ccc3o1)C2)C1CCS(=O)(=O)C1] | 3-carboxylic acid | Hz, 1 H), 7.40 (dd, J = 2.08, 8.68 Hz, 1 H), 4.08 (sxt, J = 7.90 Hz, 1 H), 3.72 (quin, J = 8.50 Hz, 1 H), 3.15-3.30 (m, 2 H), 3.00-3.14 (m, 3 H), 2.38-2.62 (m, 5 H), 2.23-2.34 (m, 2 H), 1.96-2.12 (m, 2 H), 1.91 (dd, J = 8.86, 11.06 Hz, 1 H). MS obsd. (ESI+) [(M + H)+]: 409.1. |
| 41 | N-(6-oxazolo[4,5-b]pyridin-2- | AMINE: Int-2 | ¹H NMR (DMSO-d₆, 400 MHz) δ: |
| | ylspiro[3.3]heptan-2-yl)-1,1- | ACID: 1,1- | 8.52 (dd, J = 1.10, 4.89 Hz, 1 H), |
| | dioxo-thiolane-3-carboxamide | dioxothiolane- | 7.78 (dd, J = 1.22, 8.07 Hz, 1 H), |
| | 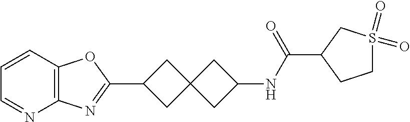 [SMILES: O=C(NC1CC2(C1)CC(c1nc3ncccc3o1)C2)C1CCS(=O)(=O)C1] | 3-carboxylic acid | 7.24-7.30 (m, 1 H), 6.28 (br d, J = 4.77 Hz, 1 H), 4.32 (sxt, J = 7.95 Hz, 1 H), 3.73 (quin, J = 8.22 Hz, 1 H), 3.18-3.37 (m, 3 H), 2.99-3.17 (m, 2 H), 2.57-2.72 (m, 4 H), 2.33- 2.55 (m, 4 H), 1.95-2.10 (m, 2 H). MS obsd. (ESI+) [(M + H)+]: 376.1. |
| 42 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-1,1- | ACID: 1,1- | 8.62 (d, J = 7.09 Hz, 1 H), 7.80 (d, J = |
| | dioxo-thiolane-2-carboxamide | dioxothiolane- | 2.20 Hz, 1 H), 7.71 (dd, J = 0.98, |
| | 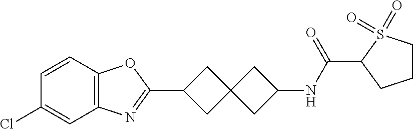 [SMILES: O=C(NC1CC2(C1)CC(c1nc3cc(Cl)ccc3o1)C2)C1CCCS1(=O)=O] | 2-carboxylic acid | 8.80 Hz, 1 H), 7.40 (dd, J = 2.20, 8.80 Hz, 1 H), 4.08 (qd, J = 8.02, 15.56 Hz, 1 H), 3.65-3.79 (m, 2 H), 3.10-3.21 (m, 1 H), 2.94 (dddd, J = 2.57, 7.52, 10.09, 12.84 Hz, 1 H), 2.39-2.61 (m, 5 H), 2.13-2.35 (m, 4 H), 2.00-2.07 (m, 1 H), 1.88- 1.99 (m, 2 H). |
| | | | MS obsd. (ESI+) [(M + H)+]: 409.0. |
| 43 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-1,1- | ACID: Int-24 | 8.38 (d, J = 7.34 Hz, 1 H), 7.79 (d, J = |
| | dioxo-1,2-thiazolidine-4- | | 2.08 Hz, 1 H), 7.71 (d, J = 8.68 |
| | carboxamide | | Hz, 1 H), 7.40 (dd, J = 2.20, 8.68 |
| | 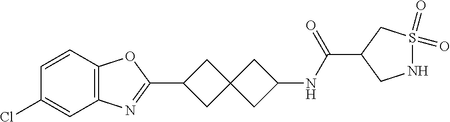 [SMILES: O=C(NC1CC2(C1)CC(c1nc3cc(Cl)ccc3o1)C2)C1CNS(=O)(=O)C1] | | Hz, 1 H), 7.05 (br s, 1 H), 4.00- 4.14 (m, 1 H), 3.66-3.77 (m, 1 H), 3.00-3.11 (m, 2 H), 2.53-2.61 (m, 2 H), 2.37-2.47 (m, 4 H), 2.23- 2.32 (m, 1 H), 1.97-2.04 (m, 1 H), 1.91 (dd, J = 8.74, 11.19 Hz, 2 H). MS obsd. (ESI+) [(M + H)+]: 410.0. |
| 44 | 2-benzyl-N-[6-(5-chloro-1,3- | AMINE: Int-1 | ¹H NMR (DMSO-d₆, 400 MHz) δ: |
| | benzoxazol-2- | ACID: Int-25 | 8.33 (d, J = 7.21 Hz, 1 H), 7.79 (d, J = |
| | yl)spiro[3.3]heptan-2-yl]-1,1- | | 2.08 Hz, 1 H), 7.71 (d, J = 8.68 |
| | dioxo-1,2-thiazolidine-4- | | Hz, 1 H), 7.27-7.42 (m, 6 H), 4.11- |
| | carboxamide | | 4.18 (m, 1 H), 3.97-4.08 (m, 2 H), |
| | 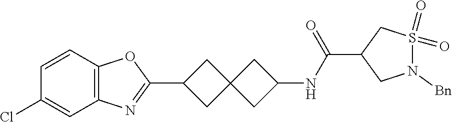 [SMILES: O=C(NC1CC2(C1)CC(c1nc3cc(Cl)ccc3o1)C2)C1CN(Cc2ccccc2)S(=O)(=O)C1] | | 3.70 (quin, J = 8.56 Hz, 1 H), 3.48- 3.56 (m, 1 H), 3.22-3.40 (m, 3 H), 2.94-3.02 (m, 1 H), 2.35-2.59 (m, 5 H), 2.20-2.30 (m, 1 H), 1.90- 2.00 (m, 1 H), 1.80-1.90 (m, 1 H). MS obsd. (ESI+) [(M + H)+]: 500.1. |
| 45 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-1,1- | ACID: 1,1- | 8.07 (br d, J = 7.09 Hz, 1 H), 7.79 |
| | dioxo-1,2-thiazolidine-3- | dioxo-1,2- | (d, J = 2.20 Hz, 1 H), 7.71 (d, J = |
| | carboxamide | thiazolidine-3- | 8.80 Hz, 1 H), 7.40 (dd, J = 2.08, |
| | | carboxylic acid | 8.68 Hz, 1 H), 7.12-7.17 (m, 1 H), |
| | 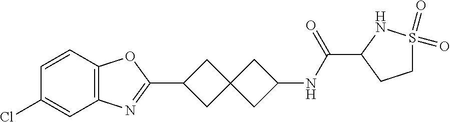 [SMILES: O=C(NC1CC2(C1)CC(c1nc3cc(Cl)ccc3o1)C2)C1CCS(=O)(=O)N1] | | 4.11 (qd, J = 8.12, 15.99 Hz, 1 H), 3.90 (q, J = 7.25 Hz, 1 H), 3.72 (quin, J = 8.31 Hz, 1 H), 3.15 (ddd, J = 5.14, 7.70, 12.59 Hz, 1 H), 3.02 (td, J = 8.44, 12.47 Hz, 1 H), 2.52- 2.62 (m, 3 H), 2.38-2.48 (m, 3 H), 2.22-2.30 (m, 1 H), 2.06-2.22 (m, 2 H), 2.01 (ddd, J = 6.24, 8.74, |
| | | | 11.19 Hz, 1 H). |
| | | | MS obsd. (ESI+) [(M + H)+]: 410.1. |
| 46 | N-[6-(5-chloro-1,3-benzoxazol- | AMINE: Int-1 | ¹H NMR (MeOH-d₄, 400 MHz) δ: |
| | 2-yl)spiro[3.3]heptan-2-yl]-2- | ACID: Int-26 | 7.63 (d, J = 2.1 Hz, 1 H), 7.55 (d, J = |
| | methyl-1,1-dioxo-1,2- | | 8.7 Hz, 1 H), 7.35 (dd, J = 8.7, 2.1 |
| | thiazolidine-3-carboxamide | | Hz, 1 H), 4.24 (quin, J = 8.2 Hz, 1 |
| | 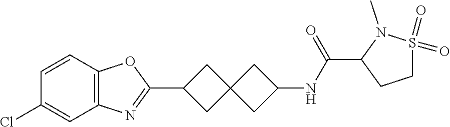 [SMILES: CN1C(C(=O)NC2CC3(C2)CC(c2nc4cc(Cl)ccc4o2)C3)CCS1(=O)=O] | | H), 3.63-3.77 (m, 2 H), 3.32-3.37 (m, 1 H), 3.13-3.21 (m, 1 H), 2.64 (s, 3 H), 2.55-2.64 (m, 4 H), 2.37- 2.53 (m, 3 H), 2.03-2.29 (m, 3 H). MS obsd. (ESI+) [(M + H)+]: 424.1. |
| 47 | N-[6-(5-chloro-1,3-benzoxazol- | AMINE: Int-1 | ¹H NMR (CDCl₃, 400 MHz) δ: 7.65 |
| | 2-yl)spiro[3.3]heptan-2-yl]-2- | ACID: 2-(1,1- | (d, J = 2.08 Hz, 1 H), 7.40 (d, J = |
| | (1,1-dioxothiolan-2- | dioxothiolan-2- | 8.56 Hz, 1 H), 7.28 (br dd, J = 2.08, |
| | yl)acetamide | yl)acetic acid | 8.56 Hz, 1 H), 5.99 (br d, J = 7.46 |
| | 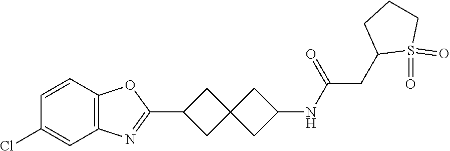 [SMILES: O=C(CC1CCCS1(=O)=O)NC1CC2(C1)CC(c1nc3cc(Cl)ccc3o1)C2] | | Hz, 1 H), 4.33 (sxt, J = 8.02 Hz, 1 H), 3.68 (quin, J = 8.56 Hz, 1 H), 3.37-3.49 (m, 1 H), 3.20 (ddd, J = 4.10, 8.47, 12.99 Hz, 1 H), 3.00 (ddd, J = 8.31, 9.72, 13.27 Hz, 1 H), 2.75 (ddd, J = 1.47, 6.66, 15.22 Hz, 1 H), 2.62-2.71 (m, 1 H), 2.43- 2.62 (m, 6 H), 2.40 (dd, J = 7.15, 15.22 Hz, 1 H), 2.08-2.27 (m, 2 H), 1.91-2.08 (m, 2 H), 1.83 (ddtd, J = |
| | | | 3.36, 6.63, 10.29, 13.69 Hz, 1 H). |
| | | | MS obsd. (ESI+) [(M + H)+]: 423.1. |
| 48 | N-(6-(5-chlorobenzo[d]oxazol- | AMINE: Int-1 | ¹H NMR (DMSO-d₆, 400 MHz) δ: |
| | 2-yl)spiro[3.3]heptan-2-yl)-3- | ACID: 3- | 7.99 (d, J = 6.97 Hz, 1 H), 7.80 (d, J = |
| | methyltetrahydrothiophene-3- | methyl-1,1- | 2.08 Hz, 1 H), 7.71 (d, J = 8.68 |
| | carboxamide 1,1-dioxide | dioxo-thiolane- | Hz, 1 H), 7.40 (dd, J = 2.08, 8.68 |
| | 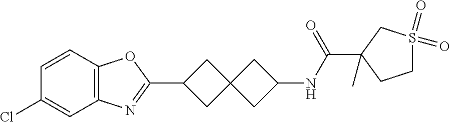 [SMILES: CC1(C(=O)NC2CC3(C2)CC(c2nc4cc(Cl)ccc4o2)C3)CCS(=O)(=O)C1] | 3-carboxylic acid | Hz, 1 H), 4.09 (sxt, J = 7.90 Hz, 1 H), 3.72 (quin, J = 8.47 Hz, 1 H), 3.61 (d, J = 13.57 Hz, 1 H), 3.22 (td, J = 6.92, 13.42 Hz, 1 H), 2.99-3.09 (m, 1 H), 2.96 (d, J = 13.45 Hz, 1 H), 2.38-2.60 (m, 5 H), 2.20-2.30 (m, 1 H), 1.93-2.14 (m, 3 H), 1.36 (s, 3 H). |
| | | | MS obsd. (ESI+) [(M + H)+]: 423.2. |
| 49 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-1,1- | ACID: 1,1- | 8.46 (br d, J = 7.09 Hz, 1 H), 7.79 |
| | dioxo-thietane-3-carboxamide | dioxothietane-3- | (d, J = 1.96 Hz, 1 H), 7.71 (d, J = |
| | 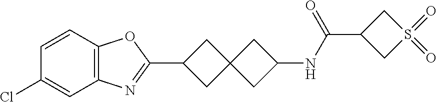 [SMILES: O=C(NC1CC2(C1)CC(c1nc3cc(Cl)ccc3o1)C2)C1CS(=O)(=O)C1] | carboxylic acid | 8.68 Hz, 1 H), 7.40 (dd, J = 2.08, 8.68 Hz, 1 H), 4.17-4.31 (m, 4 H), 4.09 (sxt, J = 7.90 Hz, 1 H), 3.72 (quin, J = 8.47 Hz, 1 H), 3.14-3.25 (m, 1 H), 2.37-2.62 (m, 5 H), 2.24- 2.32 (m, 1 H), 1.97-2.05 (m, 1 H), 1.92 (dd, J = 8.74, 11.19 Hz, 1 H). |
| | | | MS obsd. (ESI+) [(M + H)+]: 395.1. |
| 50 | N-[6-(5-chloro-1,3-benzoxazol- | AMINE: Int-1 | ¹H NMR (CDCl₃, 400 MHz) δ: 7.65 |
| | 2-yl)spiro[3.3]heptan-2-yl]-2- | ACID: 2-(1,1- | (d, J = 1.96 Hz, 1 H), 7.40 (d, J = |
| | (1,1-dioxothietan-3- | dioxothietan-3- | 8.56 Hz, 1 H), 7.27-7.30 (m, 1 H), |
| | yl)acetamide | yl)acetic acid | 5.63 (br d, J = 6.36 Hz, 1 H), 4.26- |
| | 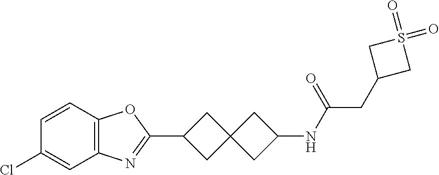 [SMILES: O=C(CC1CS(=O)(=O)C1)NC1CC2(C1)CC(c1nc3cc(Cl)ccc3o1)C2] | | 4.41 (m, 3 H), 3.79 (dd, J = 5.26, 14.67 Hz, 2 H), 3.68 (quin, J = 8.53 Hz, 1 H), 2.92-3.06 (m, 1 H), 2.53- 2.71 (m, 6 H), 2.38-2.53 (m, 2 H), 1.86-2.04 (m, 2 H). MS obsd. (ESI+) [(M + H)+]: 409.1. |
| 51 | N-[6-(5-chloro-1,3-benzoxazol- | AMINE: Int-1 | ¹H NMR (DMSO-d₆, 400 MHz) δ: |
| | 2-yl)spiro[3.3]heptan-2- | ACID: 1- | 7.68 (d, J = 1.96 Hz, 1 H), 7.43 (d, J = |
| | yl]oxetane-3-carboxamide | methylsulfonyl- | 8.68 Hz, 1 H), 7.31 (dd, J = 1.96, |
| | 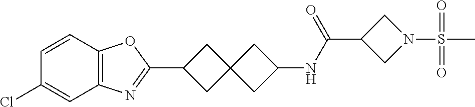 [SMILES: CS(=O)(=O)N1CC(C(=O)NC2CC3(C2)CC(c2nc4cc(Cl)ccc4o2)C3)C1] | azetidine-3- carboxylic acid | 8.68 Hz, 1 H), 5.74 (br d, J = 7.09 Hz, 1 H), 4.35 (sxt, J = 7.92 Hz, 1 H), 4.08-4.16 (m, 4 H), 3.72 (quin, J = 8.50 Hz, 1 H), 3.19 (quin, J = 7.52 Hz, 1 H), 2.98 (s, 3 H), 2.56- 2.73 (m, 4 H), 2.42-2.56 (m, 2 H), 1.93-2.08 (m, 2 H). |
| | | | MS obsd. (ESI+) [(M + H)+]: 424.1. |
| 52 | N-[6-(5-chloro-1,3-benzoxazol- | AMINE: Int-1 | ¹H NMR (CDCl₃, 400 MHz) δ: 7.65 |
| | 2-yl)spiro[3.3]heptan-2-yl]-2,2- | ACID: 2,2- | (d, J = 1.96 Hz, 1 H), 7.40 (d, J = |
| | dioxo-2-thiaspiro[3.3]heptane- | dioxo- | 8.68 Hz, 1 H), 7.29 (dd, J = 2.0, 8.4 |
| | 6-carboxamide | 2lambda6- | Hz, 1 H), 5.46 (br d, J = 7.46 Hz, 1 |
| | 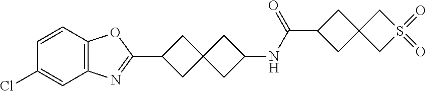 [SMILES: O=C(NC1CC2(C1)CC(c1nc3cc(Cl)ccc3o1)C2)C1CC2(C1)CS(=O)(=O)C2] | thiaspiro[3.3]hep- tane-6- carboxylic acid | H), 4.27-4.39 (m, 1 H), 4.20 (s, 2 H), 4.15 (s, 2 H), 3.68 (quin, J = 8.53 Hz, 1 H), 2.82-2.92 (m, 1 H), 2.39-2.71 (m, 10 H), 1.87-2.02 (m, 2 H). MS obsd. (ESI+) [(M + H)+]: 435.1. |
| 53 | N-[6-(5-chloro-1,3-benzoxazol- | AMINE: Int-1 | ¹H NMR (CDCl₃, 400 MHz) δ: 7.65 |
| | 2-yl)spiro[3.3]heptan-2-yl]-1,1- | ACID: 1,1- | (d, J = 1.96 Hz, 1 H), 7.39 (d, J = |
| | dioxo-thiane-4-carboxamide | dioxothiane-4- | 8.68 Hz, 1 H), 7.27-7.31 (m, 1 H), |
| | 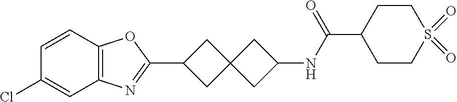 [SMILES: O=C(NC1CC2(C1)CC(c1nc3cc(Cl)ccc3o1)C2)C1CCS(=O)(=O)CC1] | carboxylic acid | 5.57 (br d, J = 6.97 Hz, 1 H), 4.32 (sxt, J = 7.95 Hz, 1 H), 3.68 (quin, J = 8.53 Hz, 1 H), 3.26-3.40 (m, 2 H), 2.96 (m, 2 H), 2.39-2.72 (m, 6 H), 2.32 (m, 5 H), 1.86-2.02 (m, 2 H). MS obsd. (ESI+) [(M + H)+]: 423.0. |
| 54 | N-(6-(5-chlorobenzo[d]oxazol- | AMINE: Int-1 | ¹H NMR (DMSO-d₆, 400 MHz) δ: |
| | 2-yl)spiro[3.3]heptan-2-yl)-4- | ACID: 4- | 7.90 (br d, J = 6.97 Hz, 1 H), 7.79 |
| | methyltetrahydro-2H- | methyl-1,1- | (d, J = 2.20 Hz, 1 H), 7.71 (d, J = |
| | thiopyran-4-carboxamide 1,1- | dioxo-thiane-4- | 8.68 Hz, 1 H), 7.40 (dd, J = 2.20, |
| | dioxide | carboxylic acid | 8.68 Hz, 1 H), 4.07-4.19 (m, 1 H), |
| | 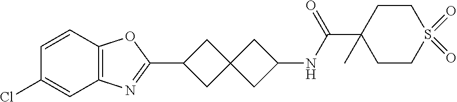 [SMILES: CC1(C(=O)NC2CC3(C2)CC(c2nc4cc(Cl)ccc4o2)C3)CCS(=O)(=O)CC1] | | 3.72 (quin, J = 8.41 Hz, 1 H), 3.01- 3.11 (m, 2 H), 2.88-2.98 (m, 2 H), 2.39-2.60 (m, 5 H), 2.35 (br d, J = 15.89 Hz, 2 H), 2.21-2.30 (m, 1 H), 2.04-2.12 (m, 1 H), 1.99 (dd, J = 8.74, 11.19 Hz, 1 H), 1.77-1.87 (m, 2 H), 1.14 (s, 3 H). |
| | | | MS obsd. (ESI+) [(M + H)+]: 437.1. |
| 55 | N-[6-(5-chloro-1,3-benzoxazol- | AMINE: Int-1 | ¹H NMR (CDCl₃, 400 MHz) δ: 7.67 |
| | 2-yl)spiro[3.3]heptan-2-yl]-2- | ACID: 2-(1,1- | (d, J = 1.96 Hz, 1 H), 7.41 (d, J = |
| | (1,1-dioxothian-3-yl)acetamide | dioxothian-3- | 8.68 Hz, 1 H), 7.30 (br d, J = 2.08 |
| | 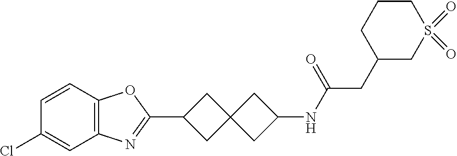 [SMILES: O=C(CC1CCCS(=O)(=O)C1)NC1CC2(C1)CC(c1nc3cc(Cl)ccc3o1)C2] | yl)acetic acid | Hz, 1 H), 5.70 (br s, 1 H), 4.27-4.40 (m, 1 H), 3.70 (quin, J = 8.47 Hz, 1 H), 3.14 (br d, J = 14.06 Hz, 1 H), 3.00-3.09 (m, 1 H), 2.84-2.97 (m, 2 H), 2.68 (br d, J = 5.38 Hz, 2 H), 2.54-2.63 (m, 3 H), 2.40-2.54 (m, 2 H), 2.27-2.39 (m, 1 H), 2.05- 2.26 (m, 3 H), 1.86-2.04 (m, 3 H), 1.31-1.46 (m, 1 H). MS obsd. (ESI+) [(M + H)+]: 437.1. |
| 56 | N-[6-(5-chloro-1,3-benzoxazol- | AMINE: Int-1 | ¹H NMR (DMSO-d₆, 400 MHz) δ: |
| | 2-yl)spiro[3.3]heptan-2-yl]-2- | ACID: 2-(1,1- | 8.10 (d, J = 7.70 Hz, 1 H), 7.79 (d, J = |
| | (1,1-dioxothiazinan-2- | dioxothiazinan- | 2.08 Hz, 1 H), 7.71 (d, J = 8.68 |
| | yl)acetamide | 2-yl)acetic acid | Hz, 1 H), 7.40 (dd, J = 2.14, 8.62 |
| | 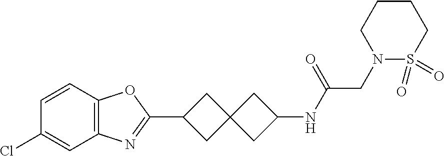 [SMILES: O=C(CN1CCCCS1(=O)=O)NC1CC2(C1)CC(c1nc3cc(Cl)ccc3o1)C2] | | Hz, 1 H), 4.13 (sxt, J = 8.04 Hz, 1 H), 3.72 (quin, J = 8.50 Hz, 1 H), 3.65 (s, 2 H), 3.33 (br d, J = 11.00 Hz, 2 H), 3.10-3.18 (m, 2 H), 2.38- 2.60 (m, 5 H), 2.20-2.29 (m, 1 H), 2.05-2.13 (m, 1 H), 1.96-2.05 (m, 3 H), 1.53-1.62 (m, 2 H). MS obsd. (ESI+) [(M + H)+]: 438.1. |
| 57 | 1,1-dioxo-N-[6-[5- | AMINE: Int-3 | ¹H NMR (CDCl₃, 400 MHz) δ: 7.95 |
| | (trifluoromethyl)-1,3- | ACID: 1,1- | (s, 1 H), 7.55-7.64 (m, 1 H), 7.27 |
| | benzoxazol-2- | dioxothiane-4- | (s, 1 H), 5.60 (br d, J = 5.87 Hz, 1 |
| | yl]spiro[3.3]heptan-2- | carboxylic acid | H), 4.26-4.42 (m, 1 H), 3.64-3.80 |
| | yl]thiane-4-carboxamide | | (m, 1 H), 3.34 (br d, J = 13.57 Hz, 2 |
| | 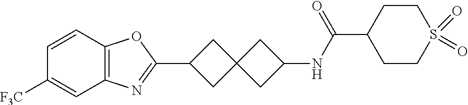 [SMILES: O=C(NC1CC2(C1)CC(c1nc3cc(C(F)(F)F)ccc3o1)C2)C1CCS(=O)(=O)CC1] | | H), 2.97 (br d, J = 11.62 Hz, 2 H), 2.41-2.75 (m, 6 H), 2.34 (br s, 5 H), 1.88-2.05 (m, 2 H). MS obsd. (ESI+) [(M + H)+]: 457.0. |
| 58 | N-[6-(5-chloro-1,3-benzoxazol- | AMINE: Int-1 | ¹H NMR (CDCl₃, 400 MHz) δ: 7.64 |
| | 2-yl)spiro[3.3]heptan-2- | SULFONYL | (d, J = 1.96 Hz, 1 H), 7.39 (d, J = |
| | yl]propane-2-sulfonamide | CHLORIDE: | 8.68 Hz, 1 H), 7.27-7.30 (m, 1 H), |
| | 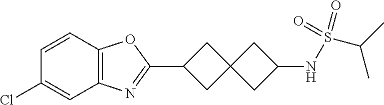 [SMILES: CC(C)S(=O)(=O)NC1CC2(C1)CC(c1nc3cc(Cl)ccc3o1)C2] | propane-2- sulfonyl chloride | 4.19 (br d, J = 8.19 Hz, 1 H), 3.88 (sxt, J = 8.24 Hz, 1 H), 3.67 (quin, J = 8.44 Hz, 1 H), 3.10 (spt, J = 6.83 Hz, 1 H), 2.63-2.73 (m, 1 H), 2.39- 2.61 (m, 5 H), 1.95-2.11 (m, 2 H), 1.36 (dd, J = 1.83, 6.85 Hz, 6 H). MS obsd. (ESI+) [(M + H)+]: 369.2. |

### Example 59

### 1-[6-(5-Chloro-1,3-benzoxazol-2-yl)spiro[3.3]heptan-2-yl]-3-ethyl-urea

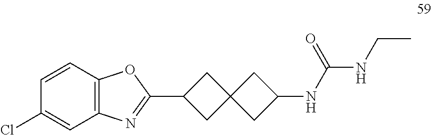 [SMILES: CCNC(=O)NC1CC2(C1)CC(c1nc3cc(Cl)ccc3o1)C2]

### Preparation of 1-[6-(5-chloro-1,3-benzoxazol-2-yl)spiro[3.3]heptan-2-yl]-3-ethyl-urea

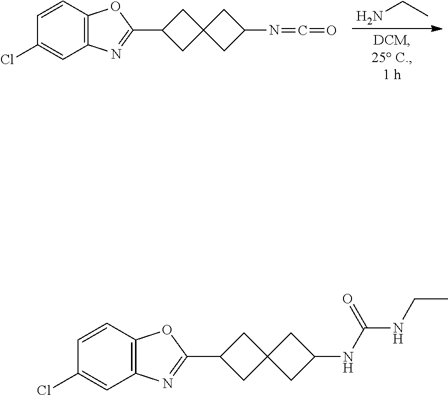

To a solution of 5-chloro-2-(2-isocyanatospiro[3.3]heptan-6-yl)-1,3-benzoxazole (Int-27) (157 mg, 0.54 mmol) in DCM (15 mL) was added ethylamine (73 mg, 1.63 mmol, as the “AMINE” in Table 2). Then the mixture was stirred at 25° C. for 1 h. The mixture was diluted with EtOAc (90 ml) and washed with water (20 mL×2). The organic layer was dried over Na₂SO₄ and concentrated. The residue was purified by prep-HPLC to give 1-[6-(5-chloro-1,3-benzoxazol-2-yl)spiro[3.3]heptan-2-yl]-3-ethyl-urea (Example 59) (24.5 mg, 0.070 mmol) as a white solid.

MS obsd. (ESI) [(M+H)⁺]: 334.0. ¹H=NMR (DMSO-d₆, 400 MHz) δ: 7.79 (d, J=1.96 Hz, 1H), 7.71 (d, J=8.56 Hz, 1H), 7.39 (dd, J=2.08, 8.68 Hz, 1H), 6.01 (d, J=8.07 Hz, 1H), 5.68 (br t, J=5.50 Hz, 1H), 3.93 (qd, J=8.08, 16.11 Hz, 1H), 3.70 (quin, J=8.38 Hz, 1H), 2.92-83.02 (m, 2H), 2.42-2.53 (m, 3H), 2.34-2.41 (m, 2H), 2.17-2.27 (m, 1H), 1.84-1.92 (i, 1H), 1.75-1.83 (m, 1H), 0.96 (t, J=7.09 Hz, 3H).

The following Example 60 to Example 68 were prepared in analogy to the procedure described for the preparation of Example 59, replacing ethylamine with “AMINE”. The “AMINE” are the reagents indicated in Table 2.

TABLE 2
Compounds synthesis and characterization

| Examples | Name and Structure | AMINE | ¹H NMR and MS obsd. (ESI⁺) |
|---|---|---|---|
| 60 | 1-[6-(5-chloro-1,3-benzoxazol- | propan-1-amine | ¹H NMR (DMSO-d₆, 400 MHz) δ: |
| | 2-yl)spiro[3.3]heptan-2-yl]-3- | | 7.62 (d, J = 2.08 Hz, 1 H), 7.54 (d, J = |
| | propyl-urea | | 8.68 Hz, 1 H), 7.35 (dd, J = 2.08, |
| | 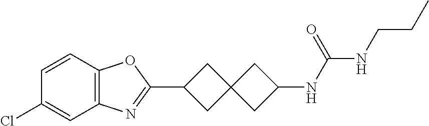 [SMILES: CCCNC(=O)NC1CC2(C1)CC(c1nc3cc(Cl)ccc3o1)C2] | | 8.68 Hz, 1 H), 4.04 (quin, J = 8.01 Hz, 1 H), 3.71 (quin, J = 8.53 Hz, 1 H), 3.05 (t, J = 7.03 Hz, 2 H), 2.31- 2.66 (m, 6 H), 1.97 (dd, J = 8.74, 10.82 Hz, 1 H), 1.89 (dd, J = 8.74, 11.31 Hz, 1 H), 1.47 (sxt, J = 7.29 Hz, 2 H), 0.90 (t, J = 7.40 Hz, 3 H). MS obsd. (ESI⁺) [(M + H)⁺]: 348.0. |
| 61 | 1-[6-(5-chloro-1,3-benzoxazol- | propan-2-amine | ¹H NMR (DMSO-d₆, 400 MHz) δ: |
| | 2-yl)spiro[3.3]heptan-2-yl]-3- | | 7.79 (d, J = 2.20 Hz, 1 H), 7.71 (d, J = |
| | isopropyl-urea | | 8.56 Hz, 1 H), 7.39 (dd, J = 2.08, |
| | 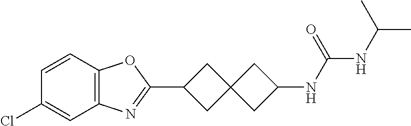 [SMILES: CC(C)NC(=O)NC1CC2(C1)CC(c1nc3cc(Cl)ccc3o1)C2] | | 8.68 Hz, 1 H), 5.91 (d, J = 8.31 Hz, 1 H), 5.54 (d, J = 7.58 Hz, 1 H), 3.95 (qd, J = 8.17, 16.08 Hz, 1 H), 3.71 (t, J = 8.56 Hz, 1 H), 3.58- 3.67 (m, 1 H), 2.35-2.57 (m, 5 H), 2.18-2.27 (m, 1 H), 1.83-1.91 (m, 1 H), 1.77 (dd, J = 8.80, 11.00 Hz, 1 H), 1.00 (d, J = 6.60 Hz, 7 H). |
| | | | MS obsd. (ESI⁺) [(M + H)⁺]: 348.0. |
| 62 | 3-[6-(5-chloro-1,3-benzoxazol- | N-methyl | ¹H NMR (CDCl₃, 400 MHz) δ: 7.79 |
| | 2-yl)spiro[3.3]heptan-2-yl]-1- | ethanamine | (d, J = 2.08 Hz, 1 H), 7.71 (d, J = |
| | ethyl-1-methyl-urea | | 8.68 Hz, 1 H), 7.40 (dd, J = 2.14, |
| | 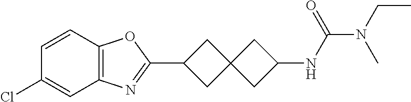 [SMILES: CCN(C)C(=O)NC1CC2(C1)CC(c1nc3cc(Cl)ccc3o1)C2] | | 8.62 Hz, 1 H), 6.28 (d, J = 7.58 Hz, 1 H), 3.95-4.08 (m, 1 H), 3.71 (quin, J = 8.50 Hz, 1 H), 3.18 (q, J = 7.05 Hz, 2 H), 2.73 (s, 3 H), 2.36- 2.58 (m, 5 H), 2.15-2.24 (m, 1 H), 2.01-2.10 (m, 1 H), 1.92-2.00 (m, 1 H), 0.96 (t, J = 7.03 Hz, 3 H). |
| | | | MS obsd. (ESI+) [(M + H)+]: 348.0. |
| 63 | 1-[6-(5-chloro-1,3-benzoxazol- | tetrahydrofuran- | ¹H NMR (DMSO-d₆, 400 MHz) δ: |
| | 2-yl)spiro[3.3]heptan-2-yl]-3- | 3-ylmethanamine | 7.79 (d, J = 2.08 Hz, 1 H), 7.71 (d, J = |
| | (tetrahydrofuran-3- | | 8.68 Hz, 1 H), 7.39 (dd, J = 2.08, |
| | ylmethyl)urea | | 8.56 Hz, 1 H), 6.04 (br d, J = 7.95 |
| | 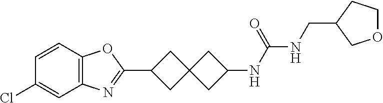 [SMILES: O=C(NCC1CCOC1)NC1CC2(C1)CC(c1nc3cc(Cl)ccc3o1)C2] | | Hz, 1 H), 5.88 (br t, J = 5.62 Hz, 1 H), 3.94 (qd, J = 7.99, 16.02 Hz, 1 H), 3.54-3.76 (m, 4 H), 3.35 (br d, J = 5.75 Hz, 1 H), 2.88-3.02 (m, 2 H), 2.34-2.58 (m, 5 H), 2.18-2.31 (m, 2 H), 1.74-1.94 (m, 3 H), 1.42- 1.54 (m, 1 H). |
| | | | MS obsd. (ESI+) [(M + H)+]: 390.0. |
| 64 | 1-[6-(5-chloro-1,3-benzoxazol- | 3- | ¹H NMR (DMSO-d₆, 400 MHz) δ: |
| | 2-yl)spiro[3.3]heptan-2-yl]-3- | (aminomethyl)cy- | 7.79 (d, J = 2.08 Hz, 1 H), 7.71 (d, J = |
| | [(3- | clobutanol | 8.68 Hz, 1 H), 7.39 (dd, J = 2.20, |
| | hydroxycyclobutyl)methyl]urea | hydrochloride | 8.68 Hz, 1 H), 6.00 (d, J = 8.07 Hz, |
| | 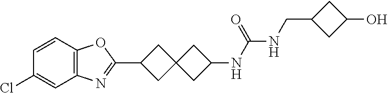 [SMILES: O=C(NCC1CC(O)C1)NC1CC2(C1)CC(c1nc3cc(Cl)ccc3o1)C2] | | 1 H), 5.66-5.79 (m, 1 H), 4.91 (d, J = 6.36 Hz, 1 H), 4.14 (qd, J = 6.81, 13.56 Hz, 0.5 H), 3.90-3.99 (m, 1 H), 3.81-3.89 (m, 0.5 H), 3.71 (quin, J = 8.50 Hz, 1 H), 2.91-3.02 (m, 2 H), 2.35-2.57 (m, 5 H), 2.12- |
| | | | 2.27 (m, 3 H), 1.74-1.91 (m, 4 H), |
| | | | 1.35-1.47 (m, 1 H). |
| | | | MS obsd. (ESI+) [(M + H)+]: 390.0. |
| 65 | 1-[6-(5-chloro-1,3-benzoxazol- | 3- | ¹H NMR (DMSO-d₆, 400 MHz) δ: |
| | 2-yl)spiro[3.3]heptan-2-yl]-3- | aminotetrahydro- | 7.79 (d, J = 1.96 Hz, 1 H), 7.71 (d, J = |
| | tetrahydrofuran-3-yl-urea | furan | 8.68 Hz, 1 H), 7.40 (dd, J = 2.14, |
| | 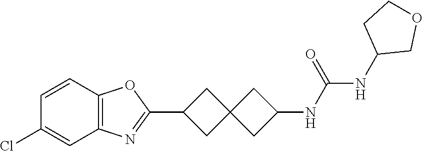 [SMILES: O=C(NC1CCOC1)NC1CC2(C1)CC(c1nc3cc(Cl)ccc3o1)C2] | | 8.62 Hz, 1 H), 5.99 (s, 1 H), 5.98 (s, 1 H), 4.04-4.12 (m, 1 H), 3.95 (sxt, J = 8.07 Hz, 1 H), 3.61-3.77 (m, 4 H), 3.36 (dt, J = 1.47, 4.40 Hz, 1 H), 2.43-2.57 (m, 3 H), 2.35-2.41 (m, 2 H), 2.19-2.27 (m, 1 H), 1.97- 2.09 (m, 1 H), 1.84-1.92 (m, 1 H), 1.79 (dd, J = 8.99, 10.94 Hz, 1 H), 1.61 (dt, J = 4.95, 12.50 Hz, 1 H). MS obsd. (ESI+) [(M + H)+]: 378.0. |
| 66 | N-[6-(5-chloro-1,3- | pyrrolidine | ¹H NMR (CDCl₃, 400 MHz) δ: 7.79 |
| | benzoxazol-2- | | (d, J = 1.96 Hz, 1 H), 7.71 (d, J = |
| | yl)spiro[3.3]heptan-2- | | 8.56 Hz, 1 H), 7.40 (dd, J = 2.20, |
| | yl]pyrrolidine-1- | | 8.68 Hz, 1 H), 6.14 (d, J = 7.82 Hz, |
| | carboxamide | | 1 H), 4.02 (sxt, J = 8.19 Hz, 1 H), |
| | 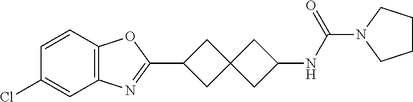 [SMILES: O=C(NC1CC2(C1)CC(c1nc3cc(Cl)ccc3o1)C2)N1CCCC1] | | 3.71 (quin, J = 8.50 Hz, 1 H), 3.12- 3.22 (m, 4 H), 2.35-2.59 (m, 5 H), 2.15-2.24 (m, 1 H), 2.02-2.10 (m, 1 H), 1.93-2.01 (m, 1 H), 1.71- 1.82 (m, 4 H). MS obsd. (ESI+) [(M + H)+]: 360.0 |
| 67 | N-[6-(5-chloro-1,3- | 2-oxa-7- | ¹H NMR (DMSO-d₆, 400 MHz) δ: |
| | benzoxazol-2- | azaspiro[3.4]oc- | 7.79 (d, J = 2.08 Hz, 1 H), 7.71 (d, J = |
| | yl)spiro[3.3]heptan-2-yl]-2- | tane; oxalic acid | 8.68 Hz, 1 H), 7.40 (dd, J = 2.20, |
| | oxa-7-azaspiro[3.4]octane-7- | | 8.68 Hz, 1 H), 6.23 (d, J = 7.82 Hz, |
| | carboxamide | | 1 H), 4.41-4.50 (m, 4 H), 4.01 (sxt, |
| | 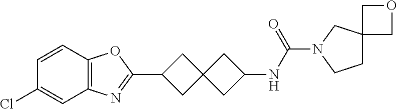 [SMILES: O=C(NC1CC2(C1)CC(c1nc3cc(Cl)ccc3o1)C2)N1CCC2(COC2)C1] | | J = 8.17 Hz, 1 H), 3.71 (quin, J = 8.50 Hz, 1 H), 3.43 (s, 2 H), 3.19 (t, J = 6.91 Hz, 2 H), 2.35-2.58 (m, 5 H), 2.15-2.24 (m, 1 H), 2.02-2.10 (m, 3 H), 1.93-2.01 (m, 1 H). MS obsd. (ESI+) [(M + H)+]: 402.0. |
| 68 | N-[6-(5-chloro-1,3- | thiomorpholine | ¹H NMR (DMSO-d₆, 400 MHz) δ: |
| | benzoxazol-2- | 1,1-dioxide | 7.79 (d, J = 2.08 Hz, 1 H), 7.71 (d, J = |
| | yl)spiro[3.3]heptan-2-yl]-1,1- | | 8.68 Hz, 1 H), 7.40 (dd, J = 2.08, |
| | dioxo-1,4-thiazinane-4- | | 8.68 Hz, 1 H), 6.94 (d, J = 7.09 Hz, |
| | carboxamide | | 1 H), 4.01 (sxt, J = 7.97 Hz, 1 H), |
| | 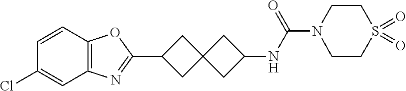 [SMILES: O=C(NC1CC2(C1)CC(c1nc3cc(Cl)ccc3o1)C2)N1CCS(=O)(=O)CC1] | | 3.65-3.79 (m, 5 H), 3.00-3.10 (m, 4 H), 2.36-2.59 (m, 5 H), 2.19- 2.28 (m, 1 H), 2.02-2.10 (m, 1 H), 1.96 (dd, J = 8.99, 11.06 Hz, 1 H). MS obsd. (ESI+) [(M + H)+]: 424.1. |

### Example 69

### N-[6-(5-chloro-1,3-benzoxazol-2-yl)spiro[3.3]heptan-2-yl]-1-methylsulfonyl-pyrrolidine-3-carboxamide

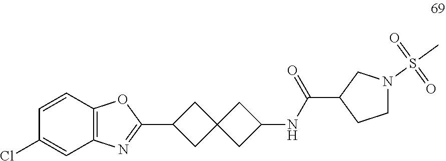 [SMILES: CS(=O)(=O)N1CCC(C(=O)NC2CC3(C2)CC(c2nc4cc(Cl)ccc4o2)C3)C1]

### Preparation of tert-butyl 3-[[6-(5-chloro-1,3-benzoxazol-2-yl)spiro[3.3]heptan-2-yl]carbamoyl]pyrrolidine-1-carboxylate

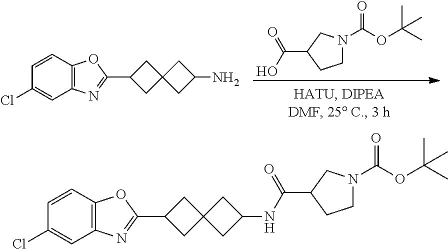 [SMILES: NC1CC2(C1)CC(c1nc3cc(Cl)ccc3o1)C2 ; CC(C)(C)OC(=O)N1CCC(C(=O)O)C1 ; CC(C)(C)OC(=O)N1CCC(C(=O)NC2CC3(C2)CC(c2nc4cc(Cl)ccc4o2)C3)C1]

To a solution of 6-(5-chloro-1,3-benzoxazol-2-yl)spiro[3.3]heptan-2-amine (Int-1) (100 mg, 0.38 mmol) and 1-tert-butoxycarbonylpyrrolidine-3-carboxylic acid (167 mg, 0.78 mmol) in DMF (1 mL) was added HATU (319 mg, 0.84 mmol) and DIPEA (0.36 mL, 2.07 mmol). Then the mixture was stirred at 25° C. for 3 h. The reaction mixture was diluted with DCM (40 ml). The solution was washed with water (10 ml×2), brine (10 ml), dried over Na₂SO₄ and concentrated in vacuum. The residue was purified by silica gel column eluted with PE/EtOAc=½ to give tert-butyl 3-[[6-(5-chloro-1,3-benzoxazol-2-yl)spiro[3.3]heptan-2-yl]carbamoyl]pyrrolidine-1-carboxylate (305 mg) as an off-white solid.

MS obsd. (ESI⁺) [(M+Na)⁺]: 481.8.

### Preparation of N-[6-(5-chloro-1,3-benzoxazol-2-yl)spiro[3.3]heptan-2-yl]pyrrolidine-3-carboxamide; 2,2,2-trifluoroacetic acid

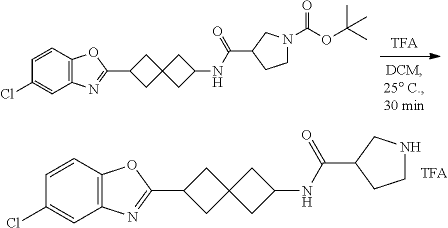 [SMILES: CC(C)(C)OC(=O)N1CCC(C(=O)NC2CC3(C2)CC(c2nc4cc(Cl)ccc4o2)C3)C1 ; O=C(NC1CC2(C1)CC(c1nc3cc(Cl)ccc3o1)C2)C1CCNC1]

To a solution of tert-butyl 3-[[6-(5-chloro-1,3-benzoxazol-2-yl)spiro[3.3]heptan-2-yl]carbamoyl]pyrrolidine-1-carboxylate (102 mg, 0.22 mmol) in DCM (4 mL) was added trifluoroacetic acid (0.4 mL, 0.22 mmol). Then the reaction was stirred for 30 min at 25° C. The mixture was concentrated to dryness in vacuo to give N-[6-(5-chloro-1,3-benzoxazol-2-yl)spiro[3.3]heptan-2-yl]pyrrolidine-3-carboxamide; 2,2,2-trifluoroacetic acid (140 mg, crude) as light yellow oil.

MS obsd. (ESI⁺) [(M+H)⁺]: 360.1.

### Preparation of N-[6-(5-chloro-1,3-benzoxazol-2-yl)spiro[3.3]heptan-2-yl]-1-methylsulfonyl-pyrrolidine-3-carboxamide

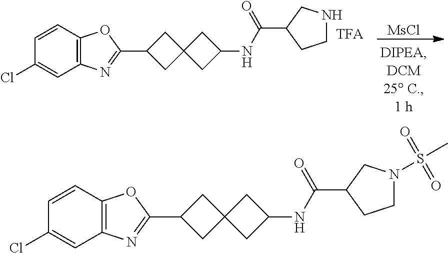 [SMILES: "MsCl" ; O=C(NC1CC2(C1)CC(c1nc3cc(Cl)ccc3o1)C2)C1CCNC1 ; CS(=O)(=O)N1CCC(C(=O)NC2CC3(C2)CC(c2nc4cc(Cl)ccc4o2)C3)C1]

To a solution of N-[6-(5-chloro-1,3-benzoxazol-2-yl)spiro[3.3]heptan-2-yl]pyrrolidine-3-carboxamide; 2,2,2-trifluoroacetic acid (140.0 mg, 0.30 mmol) in DCM (4 mL) was added DIPEA (0.26 mL, 1.48 mmol). After stirring at 25° C. for 1 h, methanesulfonyl chloride (0.03 mL, 0.38 mmol, as the “SULFONYL CHLORIDE” or “ACYL CHLORIDE” in Table 3) was added and the mixture was stirred at 25° C. for 1 h. The reaction solution was diluted with DCM (60 ml) and washed with water (15 ml×2) and brine (15 ml). The organic layer was dried over Na₂SO₄ and concentrated in vacuum. The residue was purified by flash column chromatography eluted with EtOAc to give N-[6-(5-chloro-1,3-benzoxazol-2-yl)spiro[3.3]heptan-2-yl]-1-methylsulfonyl-pyrrolidine-3-carboxamide (Example 69) (27.8 mg, 0.060 mmol) as a white solid.

MS obsd. (ESI⁺) [(M+H)⁺]: 438.1. ¹H NMR (DMSO-d₆, 400 MHz) δ: 7.64 (d, J=1.96 Hz, 1H), 7.39 (d, J=8.68 Hz, 1H), 7.25-7.30 (m, 1H), 5.72 (br d, J=7.21 Hz, 1H), 4.24-4.37 (m, 1H), 3.68 (quin, J=8.53 Hz, 1H), 3.54-3.61 (m, 1H), 3.36-3.53 (m, 3H), 2.90 (d, J=0.73 Hz, 3H), 2.82-2.89 (m, 1H), 2.62-2.71 (m, 1H), 2.53-2.62 (m, 3H), 2.38-2.52 (m, 2H), 2.09-2.27 (m, 2H), 1.88-2.04 (m, 2H).

The following Example 70 to Example 80 were prepared in analogy to the procedure described for the preparation of Example 69, replacing methanesulfonyl chloride with “SULFONYL CHLORIDE” or “ACYL CHLORIDE”. The “SULFONYL CHLORIDE” or “ACYL CHLORIDE” are the reagents indicated in Table 3.

TABLE 3
Compounds synthesis and characterization

| | | “SULFONYL | |
|---|---|---|---|
| | | CHLORIDE” or | |
| Exam- | | “ACYL | |
| ples | Name and Structure | CHLORIDE” | ¹H NMR and MS obsd. (ESI⁺) |
| 70 | N-[6-(5-chloro-1,3- | ethanesulfonyl | ¹H NMR (MeOH-d₄, 400 MHz) δ: |
| | benzoxazol-2- | chloride | 7.64 (d, J = 2.0 Hz, 1 H), 7.56 (d, J = |
| | yl)spiro[3.3]heptan-2-yl]-1- | | 8.7 Hz, 1 H), 7.36 (dd, J = 2.1, 8.6 |
| | ethylsulfonyl-pyrrolidine-3- | | Hz, 1 H), 4.26-4.16 (m, 1 H), 3.74 |
| | carboxamide | | (quin, J = 8.5 Hz, 1 H), 3.62-3.55 |
| | 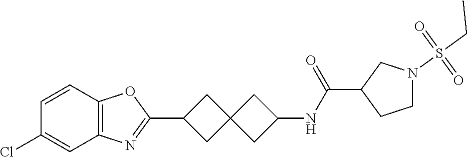 [SMILES: CCS(=O)(=O)N1CCC(C(=O)NC2CC3(C2)CC(c2nc4cc(Cl)ccc4o2)C3)C1] | | (m, 1 H), 3.53-3.36 (m, 3 H), 3.12 (ddt, J = 1.2, 6.9, 7.7 Hz, 2 H), 3.07- 2.97 (m, 1 H), 2.65-2.39 (m, 6 H), 2.19-1.98 (m, 4 H), 1.35 (dt, J = 1.0, 7.4 Hz, 3 H). MS obsd. (ESI⁺) [(M + H)⁺]: 452.1. |
| 71 | N-[6-(5-chloro-1,3- | propane-1- | ¹H NMR (MeOH-d₄, 400 MHz) δ: |
| | benzoxazol-2- | sulfonyl chloride | 7.65 (d, J = 1.8 Hz, 1 H), 7.56 (d, J = |
| | yl)spiro[3.3]heptan-2-yl]-1- | | 8.7 Hz, 1 H), 7.37 (dd, J = 2.1, 8.7 |
| | propylsulfonyl-pyrrolidine-3- | | Hz, 1 H), 4.21 (t, J = 7.9 Hz, 1 H), |
| | carboxamide | | 3.79-3.69 (m, 1 H), 3.61-3.54 (m, |
| | 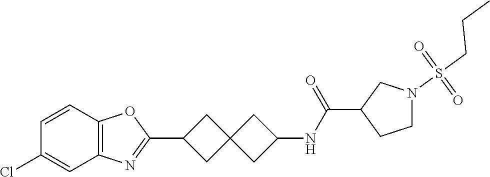 [SMILES: CCCS(=O)(=O)N1CCC(C(=O)NC2CC3(C2)CC(c2nc4cc(Cl)ccc4o2)C3)C1] | | 1 H), 3.51-3.35 (m, 3 H), 3.10- 2.97 (m, 3 H), 2.66-2.39 (m, 6 H), 2.23-1.96 (m, 4 H), 1.89-1.77 (m, 2 H), 1.09 (dt, J = 1.3, 7.5 Hz, 3 H). MS obsd. (ESI+) [(M + H)+]: 466.1. |
| 72 | N-[6-(5-chloro-1,3- | cyclopropanesul- | ¹H NMR (MeOH-d₄, 400 MHz) δ: |
| | benzoxazol-2- | fonyl chloride | 7.65 (d, J = 2.1 Hz, 1 H), 7.57 (d, J = |
| | yl)spiro[3.3]heptan-2-yl]-1- | | 8.6 Hz, 1 H), 7.37 (dd, J = 2.1, 8.7 |
| | cyclopropylsulfonyl- | | Hz, 1 H), 4.22 (t, J = 7.9 Hz, 1 H), |
| | pyrrolidine-3- | | 3.74 (t, J = 8.5 Hz, 1 H), 3.63-3.57 |
| | carboxamide | | (m, 1 H), 3.56-3.37 (m, 3 H), 3.03 |
| | 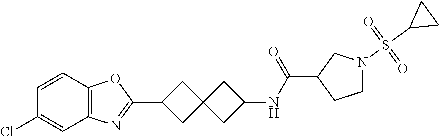 [SMILES: O=C(NC1CC2(C1)CC(c1nc3cc(Cl)ccc3o1)C2)C1CCN(S(=O)(=O)C2CC2)C1] | | (t, J = 7.7 Hz, 1 H), 2.69-2.57 (m, 4 H), 2.56-2.47 (m, 2 H), 2.46-2.38 (m, 1 H), 2.20-2.01 (m, 4 H), 1.13- 1.00 (m, 4 H). MS obsd. (ESI⁺) [(M + H)⁺]: 464.1. |
| 73 | 1-acetyl-N-[6-(5-chloro-1,3- | acetyl chloride | ¹H NMR (MeOH-d₄, 400 MHz) δ: |
| | benzoxazol-2- | | 7.53 (d, J = 2.1 Hz, 1 H), 7.45 (d, J = |
| | yl)spiro[3.3]heptan-2- | | 8.7 Hz, 1 H), 7.25 (dd, J = 2.1, 8.7 |
| | yl]pyrrolidine-3- | | Hz, 1 H), 4.10 (dt, J = 3.2, 7.9 Hz, 1 |
| | carboxamide | | H), 3.67-3.47 (m, 4 H), 3.45-3.33 |
| | 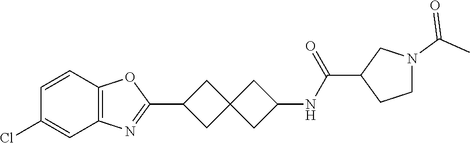 [SMILES: CC(=O)N1CCC(C(=O)NC2CC3(C2)CC(c2nc4cc(Cl)ccc4o2)C3)C1] | | (m, 1 H), 3.31-3.24 (m, 1 H), 2.98- 2.80 (m, 1 H), 2.56-2.46 (m, 3 H), 2.45-2.35 (m, 2 H), 2.34-2.26 (m, 1 H), 2.09-1.99 (m, 2 H), 1.97- 1.88 (m, 4 H). MS obsd. (ESI⁺) [(M + H)⁺]: 402.2. |
| 74 | N-[6-(5-chloro-1,3- | cyclopropanecar- | ¹H NMR (MeOH-d₄, 400 MHz) δ: |
| | benzoxazol-2- | bonyl chloride | 7.53 (d, J = 2.1 Hz, 1 H), 7.45 (d, J = |
| | yl)spiro[3.3]heptan-2-yl]-1- | | 8.7 Hz, 1 H), 7.25 (dd, J = 2.1, 8.7 |
| | (cyclopropanecarbonyl)pyr- | | Hz, 1 H), 4.11 (dt, J = 5.7, 7.8 Hz, 1 |
| | rolidine-3-carboxamide | | H), 3.84-3.74 (m, 1 H), 3.70-3.50 |
| | 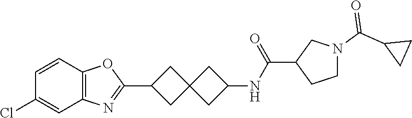 [SMILES: O=C(NC1CC2(C1)CC(c1nc3cc(Cl)ccc3o1)C2)C1CCN(C(=O)C2CC2)C1] | | (m, 3 H), 3.41-3.24 (m, 1 H), 3.01- 2.80 (m, 1 H), 2.56-2.45 (m, 3 H), 2.43-2.26 (m, 3 H), 2.14-2.06 (m, 1 H), 2.03-1.87 (m, 3 H), 1.74- 1.64 (m, 1 H), 0.83-0.68 (m, 4 H). MS obsd. (ESI⁺) [(M + H)⁺]: 428.2. |
| 75 | N-[6-(5-chloro-1,3- | cyclobutanecar- | ¹H NMR (MeOH-d₄, 400 MHz) δ: |
| | benzoxazol-2- | bonyl chloride | 7.53 (d, J = 2.1 Hz, 1 H), 7.45 (d, J = |
| | yl)spiro[3.3]heptan-2-yl]-1- | | 8.7 Hz, 1 H), 7.25 (dd, J = 2.1, 8.7 |
| | (cyclobutanecarbonyl)pyrrol- | | Hz, 1 H), 4.09 (dd, J = 7.6, 8.7 Hz, 1 |
| | idine-3-carboxamide | | H), 3.62 (t, J = 8.5 Hz, 1 H), 3.56- |
| | 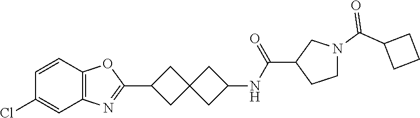 [SMILES: O=C(NC1CC2(C1)CC(c1nc3cc(Cl)ccc3o1)C2)C1CCN(C(=O)C2CCC2)C1] | | 3.45 (m, 2 H), 3.44-3.24 (m, 2 H), 2.93-2.77 (m, 1 H), 2.55-2.27 (m, 6 H), 2.21-1.85 (m, 10 H), 1.80- 1.69 (m, 1 H). MS obsd. (ESI⁺) [(M + H)⁺]: 442.2. |
| 76 | N-[6-(5-chloro-1,3- | 4- | ¹H NMR (MeOH-d₄, 400 MHz) δ: |
| | benzoxazol-2- | methylbenzene- | 7.73 (d, J = 8.1 Hz, 2 H), 7.65 (d, J = |
| | yl)spiro[3.3]heptan-2-yl]-2- | sulfonyl chloride | 2.0 Hz, 1 H), 7.56 (d, J = 8.7 Hz, |
| | [1-(p- | | 1 H), 7.44 (d, J = 8.3 Hz, 2 H), 7.37 |
| | tolylsulfonyl)pyrrolidin-3- | | (dd, J = 2.1, 8.7 Hz, 1 H), 4.16 (t, J = |
| | yl]acetamide | | 8.0 Hz, 1 H), 3.73 (dt, J = 1.5, 8.5 |
| | 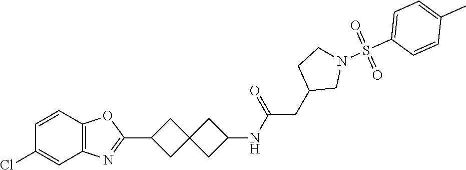 [SMILES: Cc1ccc(S(=O)(=O)N2CCC(CC(=O)NC3CC4(C3)CC(c3nc5cc(Cl)ccc5o3)C4)C2)cc1] | | Hz, 1 H), 3.45-3.35 (m, 2 H), 3.23- 3.15 (m, 1 H), 2.91-2.85 (m, 1 H), 2.65-2.56 (m, 3 H), 2.54-2.48 (m, 1 H), 2.46 (s, 3 H), 2.42-2.32 (m, 2 H), 2.13 (dd, J = 2.9, 7.5 Hz, 2 H), 2.09-2.01 (m, 1 H), 2.00-1.89 (m, 2 H). MS obsd. (ESI⁺) [(M + H)⁺]: 528.2. |
| 77 | N-[6-(5-chloro-1,3- | methanesulfonyl | ¹H NMR (MeOH-d₄, 400 MHz) δ: |
| | benzoxazol-2- | chloride | 7.65 (d, J = 2.1 Hz, 1 H), 7.56 (d, J = |
| | yl)spiro[3.3]heptan-2-yl]-2- | | 8.7 Hz, 1 H), 7.36 (dd, J = 2.1, 8.6 |
| | (1-methylsulfonylazetidin-3- | | Hz, 1 H), 4.49 (quin, J = 8.6 Hz, 1 |
| | yl)acetamide | | H), 3.75 (quin, J = 8.5 Hz, 1 H), |
| | 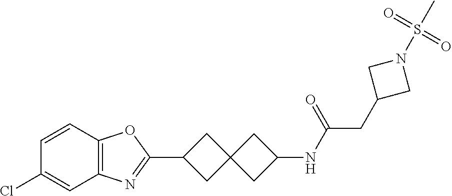 [SMILES: CS(=O)(=O)N1CC(CC(=O)NC2CC3(C2)CC(c2nc4cc(Cl)ccc4o2)C3)C1] | | 3.63 (td, J = 7.2, 9.8 Hz, 1 H), 3.38 (td, J = 5.0, 8.2 Hz, 1 H), 3.14-3.08 (m, 2 H), 2.96 (d, J = 2.7 Hz, 3 H), 2.70-2.19 (m, 11 H). MS obsd. (ESI⁺) [(M + H)⁺]: 438.1. |
| 78 | N-[6-(5-chloro-1,3- | 4- | ¹H NMR (MeOH-d₄, 400 MHz) δ: |
| | benzoxazol-2- | methylbenzene- | 7.76-7.71 (m, 2 H), 7.64 (d, J = 2.1 |
| | yl)spiro[3.3]heptan-2-yl]-2- | sulfonyl chloride | Hz, 1 H), 7.56 (d, J = 8.7 Hz, 1 H), |
| | [1-(p-tolylsulfonyl)azetidin-3- | | 7.50 (d, J = 8.1 Hz, 2 H), 7.36 (dd, J = |
| | yl]acetamide | | 2.1, 8.7 Hz, 1 H), 4.16-4.05 (m, 1 |
| | 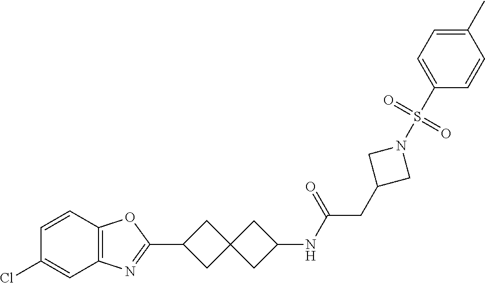 [SMILES: Cc1ccc(S(=O)(=O)N2CC(CC(=O)NC3CC4(C3)CC(c3nc5cc(Cl)ccc5o3)C4)C2)cc1] | | H), 3.86 (t, J = 8.2 Hz, 2 H), 3.72 (t, J = 8.5 Hz, 1 H), 3.45 (dd, J = 6.2, 8.1 Hz, 2 H), 2.76-2.66 (m, 1 H), 2.65-2.53 (m, 3 H), 2.52-2.40 (m, 4 H), 2.40-2.32 (m, 1 H), 2.26 (d, J = 7.7 Hz, 2 H), 2.07-1.90 (m, 2 H). MS obsd. (ESI⁺) [(M + H)⁺]: 514.2. |
| 79 | N-[6-(5-chloro-1,3- | methanesulfonyl | ¹H NMR (MeOH-d₄, 400 MHz) δ: |
| | benzoxazol-2- | chloride | 7.65 (d, J = 2.1 Hz, 1 H), 7.40 (d, J = |
| | yl)spiro[3.3]heptan-2-yl ]-2- | | 8.7 Hz, 1 H), 7.30-7.26 (m, 1 H), |
| | (1-methylsulfonyl-4- | | 4.40-4.29 (m, 1H), 3.81 (br d, J = |
| | piperidyl)acetamide | | 11.2 Hz, 2 H), 2.78 (s, 3 H), 2.73- |
| | 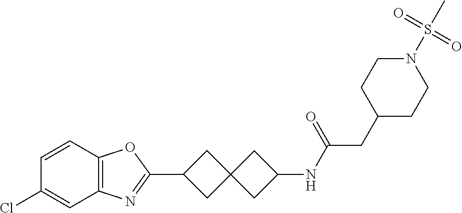 [SMILES: CS(=O)(=O)N1CCC(CC(=O)NC2CC3(C2)CC(c2nc4cc(Cl)ccc4o2)C3)CC1] | | 2.72 (m, 1 H), 2.68-2.43 (m, 8 H), 2.09 (d, J = 7.1 Hz, 2 H), 2.04-1.91 (m, 3 H), 1.88-1.80 (m, 2 H), 1.40- 1.32 (m, 2 H). MS obsd. (ESI⁺) [(M + H)⁺]: 466.1. |
| 80 | N-[6-(5-chloro-1,3- | 4- | ¹H NMR (MeOH-d₄, 400 MHz) δ: |
| | benzoxazol-2- | methylbenzene- | 7.68-7.63 (m, 3 H), 7.56 (d, J = 8.7 |
| | yl)spiro[3.3]heptan-2-yl]-2- | sulfonyl chloride | Hz, 1 H), 7.43 (d, J = 7.9 Hz, 2 H), |
| | [1-(p-tolylsulfonyl)-4- | | 7.36 (dd, J = 2.1, 8.6 Hz, 1 H), 4.22- |
| | piperidyl]acetamide | | 4.13 (m, 1 H), 3.76-3.69 (m, 3 H), |
| | 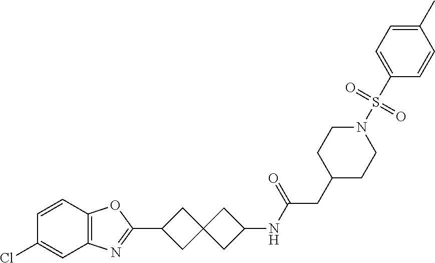 [SMILES: Cc1ccc(S(=O)(=O)N2CCC(CC(=O)NC3CC4(C3)CC(c3nc5cc(Cl)ccc5o3)C4)CC2)cc1] | | 2.65-2.56 (m, 3 H), 2.48-2.48 (m, 1 H), 2.55-2.47 (m, 1 H), 2.46 (s, 3 H), 2.43-2.34 (m, 1 H), 2.27 (dt, J = 2.1, 12.0 Hz, 2 H), 2.09-2.05 (m, 3 H), 2.04-1.94 (m, 1 H), 1.76- 1.61 (m, 3 H), 1.34 (br d, J = 4.5 Hz, 2 H). MS obsd. (ESI⁺) [(M + H)⁺]: 542.2. |
| 81 | N-[4-[[6-(5-chloro-1,3- | cyclobutanecar- | ¹H NMR (DMSO-d₆, 400 MHz) δ: |
| | benzoxazol-2- | bonyl chloride | 11.46 (br s, 1 H), 8.07 (d, J = 7.46 |
| | yl)spiro[3.3]heptan-2- | | Hz, 1 H), 7.79 (d, J = 2.08 Hz, 1 H), |
| | yl]amino]-4-oxo- | | 7.71 (d, J = 8.56 Hz, 1 H), 7.39 (dd, |
| | butyl]sulfonylcyclobutanecar- | | J = 2.20, 8.68 Hz, 1 H), 4.07 (sxt, J = |
| | boxamide | | 8.00 Hz, 1 H), 3.71 (quin, J = 8.47 |
| | 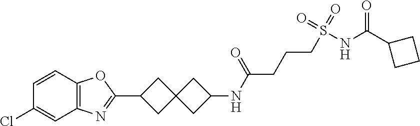 [SMILES: O=C(CCCS(=O)(=O)NC(=O)C1CCC1)NC1CC2(C1)CC(c1nc3cc(Cl)ccc3o1)C2] | | Hz, 1 H), 3.29 (br s, 2 H), 3.12 (quin, J = 8.34 Hz, 1 H), 2.36-2.59 (m, 5 H), 2.20-2.29 (m, 1 H), 2.02- 2.19 (m, 6 H), 1.97 (dd, J = 8.80, 10.76 Hz, 1 H), 1.69-1.93 (m, 5 H). MS obsd. (ESI⁺) [(M + H)⁺]: 494.1. |

### Example 82-a, Example 82-b

### (Rₐ)—N-[6-(5-chloro-1,3-benzoxazol-2-yl)spiro[3.3]heptan-2-yl]-1,1-dioxo-thiane-4-carboxamide

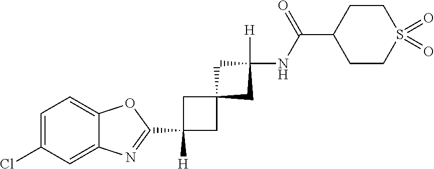

### (Sₐ)—N-[6-(5-chloro-1,3-benzoxazol-2-yl)spiro[3.3]heptan-2-yl]-1,1-dioxo-thiane-4-carboxamide

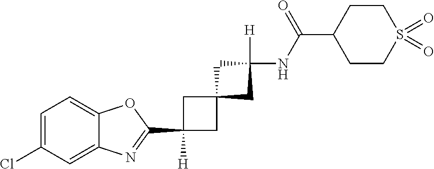

The two enantiomers (Example 82-a, Example 82-b) were obtained through SFC [Instrument: SFC 80, Column: ChiralCel OJ, 250×20 mm I.D., 5 μm; Mobile phase: A for CO₂ and B for Methanol (0.1% NH₃H₂O); Gradient: B 15%; Flow rate: 50 mL/min; Back pressure: 100 bar; Column temperature: 35° C.; elution order was Example 82-a, Example 82-b] chiral separation of N-[6-(5-chloro-1,3-benzoxazol-2-yl)spiro[3.3]heptan-2-yl]-1,1-dioxo-thiane-4-carboxamide (Example 53).

Example 82-a: MS obsd. (ESI⁺) [(M+H)⁺]: 423.1. ¹H NMR (DMSO-d₆, 400 MHz) δ: 8.15 (d, J=7.34 Hz, 1H), 7.79 (d, J=2.08 Hz, 1H), 7.71 (d, J=8.68 Hz, 1H), 7.39 (dd, J=2.20, 8.68 Hz, 1H), 3.99-4.12 (m, 1H), 3.71 (quin, J=8.47 Hz, 1H), 3.03-3.20 (m, 4H), 2.42-2.59 (m, 3H), 2.32-2.43 (m, 3H), 2.20-2.29 (m, 1H), 1.98-2.10 (m, 4H), 1.86-1.98 (m, 2H).

[α]²⁰_{D}=−7.567 (C=0.1, MeOH).

Example 82-b: MS obsd. (ESI⁺) [(M+H)⁺]: 423.1. ¹H NMR (DMSO-d₆, 400 MHz) δ: 8.15 (d, J=7.34 Hz, 1H), 7.79 (d, J=2.08 Hz, 1H), 7.71 (d, J=8.68 Hz, 1H), 7.39 (dd, J=2.20, 8.68 Hz, 1H), 4.07 (sxt, J=7.95 Hz, 1H), 3.71 (quin, J=8.50 Hz, 1H), 3.01-3.19 (m, 4H), 2.43-2.60 (m, 3H), 2.31-2.43 (m, 3H), 2.20-2.30 (m, 1H), 1.98-2.08 (m, 4H), 1.86-1.98 (m, 2H).

[α]²⁰_{D}=+7.359 (C=0.1, MeOH).

### Example 83

### N-[6-(5-chloro-1,3-benzoxazol-2-yl)spiro[3.3]heptan-2-yl]-2-cyclopropylsulfonyl-pyridine-4-carboxamide

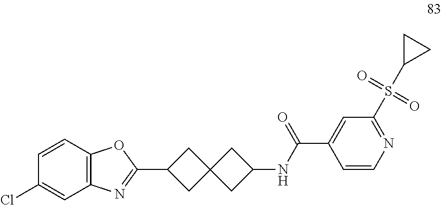 [SMILES: O=C(NC1CC2(C1)CC(c1nc3cc(Cl)ccc3o1)C2)c1ccnc(S(=O)(=O)C2CC2)c1]

### Preparation of N-[6-(5-chloro-1,3-benzoxazol-2-yl)spiro[3.3]heptan-2-yl]-2-cyclopropylsulfanyl-pyridine-4-carboxamide

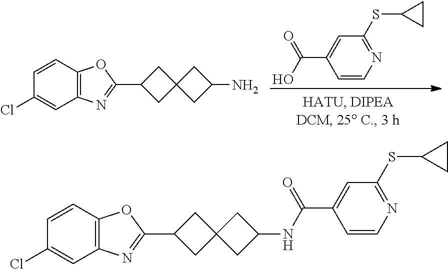 [SMILES: NC1CC2(C1)CC(c1nc3cc(Cl)ccc3o1)C2 ; O=C(O)c1ccnc(SC2CC2)c1 ; O=C(NC1CC2(C1)CC(c1nc3cc(Cl)ccc3o1)C2)c1ccnc(SC2CC2)c1]

To a solution of 2-cyclopropylsulfanylpyridine-4-carboxylic acid (Int-28) (160 mg, 0.82 mmol) in DCM (5 mL) was added T₃P (782 mg, 2.46 mmol), 6-(5-chloro-1,3-benzoxazol-2-yl)spiro[3.3]heptan-2-amine (Int-1) (236 mg, 0.90 mmol) and triethylamine (0.34 mL, 2.46 mmol). Then the mixture was stirred at 25° C. for 3 h. The reaction was diluted with DCM (20 ml). The solution was washed with water (10 ml×2), brine (10 ml) and dried over Na₂SO₄ and concentrated in vacuum. The residue was purified by silica gel column eluted with PE/EtOAc=1/1 to give N-[6-(5-chloro-1,3-benzoxazol-2-yl)spiro[3.3]heptan-2-yl]-2-cyclopropylsulfanyl-pyridine-4-carboxamide (180 mg) as a light yellow solid.

MS obsd. (ESI⁺) [(M+H)⁺]: 440.1.

### Preparation of N-[6-(5-chloro-1,3-benzoxazol-2-yl)spiro[3.3]heptan-2-yl]-2-cyclopropylsulfonyl-pyridine-4-carboxamide

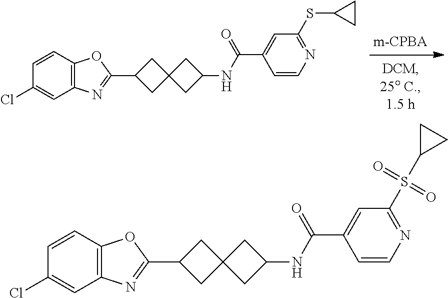 [SMILES: O=C(NC1CC2(C1)CC(c1nc3cc(Cl)ccc3o1)C2)c1ccnc(SC2CC2)c1 ; O=C(NC1CC2(C1)CC(c1nc3cc(Cl)ccc3o1)C2)c1ccnc(S(=O)(=O)C2CC2)c1]

To a solution of N-[6-(5-chloro-1,3-benzoxazol-2-yl)spiro[3.3]heptan-2-yl]-2-cyclopropylsulfanyl-pyridine-4-carboxamide (80 mg, 0.18 mmol) in DCM (5 mL) was added 3-chloroperbenzoic acid (69 mg, 0.73 mmol). The reaction mixture was stirred at 25° C. for 1.5 h. The mixture was quenched with water. The residue was extracted with DCM (15 mL×3). The organic layers were washed with brine (10 mL×3), dried over Na₂SO₄ and concentrated in vacuum. The residue was purified by pre-HPLC to give N-[6-(5-chloro-1,3-benzoxazol-2-yl)spiro[3.3]heptan-2-yl]-2-cyclopropylsulfonyl-pyridine-4-carboxamide (Example 83) (31 mg) as a light yellow solid.

MS obsd. (ESI⁺) [(M+H)⁺]: 472.1. ¹H NMR (DMSO-d₆, 400 MHz) δ: 9.20 (d, J=7.2 Hz, 1H), 8.94-8.98 (m, 1H), 8.36-8.40 (m, 1H), 8.10 (dd, J=5.0, 1.7 Hz, 1H), 7.81 (d, J=2.1 Hz, 1H), 7.72 (d, J=8.7 Hz, 1H), 7.40 (dd, J=8.6, 2.1 Hz, 1H), 4.35 (sxt, J=8.0 Hz, 1H), 3.76 (quin, J=8.5 Hz, 1H), 3.00 (tt, J=7.7, 5.0 Hz, 1H), 2.52-2.68 (m, 3H), 2.33-2.48 (m, 3H), 2.12-2.28 (m, 2H), 1.07-1.18 (m, 4H).

### Example 84-a, Example 84-b

### (Rₐ)—N-[6-(5-chloro-1,3-benzoxazol-2-yl)spiro[3.3]heptan-2-yl]-2-cyclopropylsulfonyl-pyridine-4-carboxamide

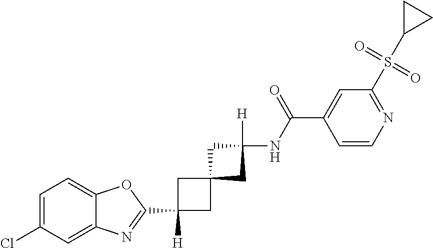

### (Sₐ)—N-[6-(5-chloro-1,3-benzoxazol-2-yl)spiro[3.3]heptan-2-yl]-2-cyclopropylsulfonyl-pyridine-4-carboxamide

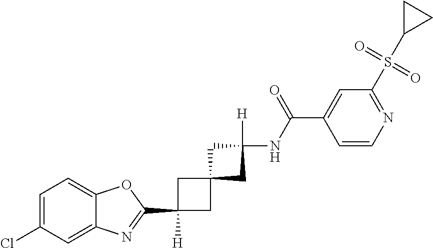

The two enantiomers (Example 84-a, Example 84-b) were obtained through SFC [Instrument: Shimadzu LC-20AT, Column: CHIRALPAK IQ 5.0 cm I.D.×25 cmL, 10 μm; Mobile phase: MeOH/ACN=90/10 (V/V); Flow rate: 60 mL/min; Wave length: UV 254 nm; Column temperature: 35° C.; elution order was Example 84-a, Example 84-b] chiral separation of N-[6-(5-chloro-1,3-benzoxazol-2-yl)spiro[3.3]heptan-2-yl]-2-cyclopropylsulfonyl-pyridine-4-carboxamide (Example 83).

Example 84-a: MS obsd. (ESI⁺) [(M+H)⁺]: 472.1. ¹H NMR (MeOH-d₄, 400 MHz) δ: 8.89 (d, J=5.0 Hz, 1H), 8.37 (s, 1H), 8.02 (dd, J=5.0, 1.5 Hz, 1H), 7.64 (d, J=2.1 Hz, 1H), 7.56 (d, J=8.7 Hz, 1H), 7.35 (dd, J=8.7, 1.8 Hz, 1H), 4.44 (quin, J=8.2 Hz, 1H), 3.76 (quin, J=8.5 Hz, 1H), 2.85-2.96 (m, 1H), 2.46-2.73 (m, 6H), 2.16-2.32 (m, 2H), 1.09-1.32 (m, 4H).

Example 84-b: MS obsd. (ESI⁺) [(M+H)⁺]: 472.1. ¹H NMR (MeOH-d₄, 400 MHz) δ: 8.89 (d, J=4.9 Hz, 1H), 8.37 (s, 1H), 8.02 (dd, J=5.0, 1.6 Hz, 1H), 7.64 (d, J=2.0 Hz, 1H), 7.56 (d, J=8.7 Hz, 1H), 7.36 (dd, J=8.7, 2.1 Hz, 1H), 4.44 (quin, J 8.2 Hz, 1H), 3.76 (quin, J=8.5 Hz, 1H), 2.87-2.95 (m, 1H), 2.45-2.74 (m, 6H), 2.16-2.31 (m, 2H), 1.23-1.31 (m, 2H), 1.07-1.18 (m, 2H).

### Example 85

### N-[6-(6-chlorooxazolo[5,4-b]pyridin-2-yl)spiro[3.3]heptan-2-yl]-2-cyclopropylsulfonyl-pyridine-4-carboxamide

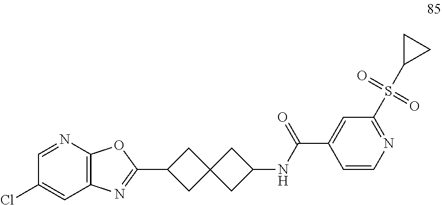 [SMILES: O=C(NC1CC2(C1)CC(c1nc3cc(Cl)cnc3o1)C2)c1ccnc(S(=O)(=O)C2CC2)c1]

The title compound was prepared in analogy to the procedure described for the preparation of Example 83, by using 6-(6-chlorooxazolo[5,4-b]pyridin-2-yl)spiro[3.3]heptan-2-amine (Int-3) instead of 6-(5-chloro-1,3-benzoxazol-2-yl)spiro[3.3]heptan-2-amine (Int-1) to afford Example 85 as an off-white solid.

MS obsd. (ESI⁺) [(M+H)⁺]: 473.1. ¹H NMR (DMSO-d₆, 400 MHz) δ: 9.07-9.36 (m, 1H), 8.85 9.04 (m, 1H), 8.34-8.43 (m, 3H), 8.02-8.18 (m, 1H), 4.30-4.46 (m, 1H), 3.80 (quin, J=8.5 Hz, 1H), 3.01 (tt, J=7.7, 5.0 Hz, 1H), 2.55-2.68 (m, 3H), 2.44-2.50 (m, 2H), 2.33-2.42 (m, 1H), 2.13-2.29 (m, 2H), 1.06-1.21 (m, 4H).

### Example 86

### N-[6-(5-chloro-1,3-benzoxazol-2-yl)spiro[3.3]heptan-2-yl]-2-(cyclopropylsulfonimidoyl)pyridine-4-carboxamide

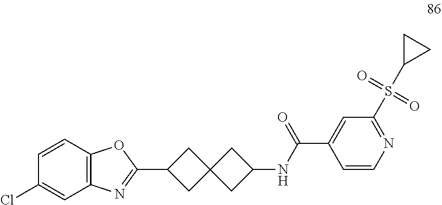 [SMILES: O=C(NC1CC2(C1)CC(c1nc3cc(Cl)ccc3o1)C2)c1ccnc(S(=O)(=O)C2CC2)c1]

### Preparation of N-[6-(5-chloro-1,3-benzoxazol-2-yl)spiro[3.3]heptan-2-yl]-2-(cyclopropylsulfonimidoyl)pyridine-4-carboxamide

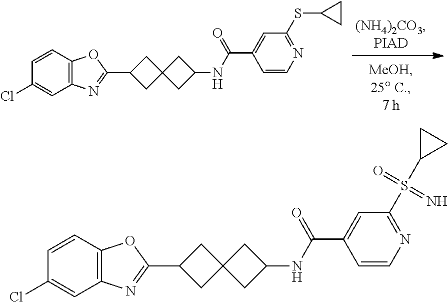 [SMILES: O=C(NC1CC2(C1)CC(c1nc3cc(Cl)ccc3o1)C2)c1ccnc(SC2CC2)c1 ; N=S(=O)(c1cc(C(=O)NC2CC3(C2)CC(c2nc4cc(Cl)ccc4o2)C3)ccn1)C1CC1]

A mixture of (NH₄)₂CO₃ (31.33 mg, 0.33 mmol) and PIAD (105.03 mg, 0.33 mmol) was stirred in methanol (5 mL) at 25° C. for 5 min. And then N-[6-(5-chloro-1,3-benzoxazol-2-yl)spiro[3.3]heptan-2-yl]-2-cyclopropylsulfanyl-pyridine-4-carboxamide (48 mg, 0.11 mmol) was added. The reaction mixture was stirred at 25° C. fo 7 h. The mixture was quenched by water. The reaction mixture was concentrated to remove organic solvents (MeOH) under reduced pressure. The residue was extracted with DCM (15 mL×3). The combined organic layers were washed with brine (10 mL×3), dried over Na₂SO₄, filtered and concentrated. The residue was purified by pre-HPLC to give N-[6-(5-chloro-1,3-benzoxazol-2-yl)spiro[3.3]heptan-2-yl]-2-(cyclopropylsulfonimidoyl)pyridine-4-carboxamide (25 mg) as a white solid.

MS obsd. (ESI⁺) [(M+H)⁺]: 471.1. ¹H NMR (DMSO-d₆, 400 MHz) δ: 9.15 (d, J=7.2 Hz, 1H), 8.88 (d, J=5.0 Hz, 1H), 8.39 (d, J=0.7 Hz, 1H), 7.99 (dd, J=5.0, 1.7 Hz, 1H), 7.81 (d, J=2.1 Hz, 1H), 7.72 (d, J=8.8 Hz, 1H), 7.40 (dd, J=8.7, 2.2 Hz, 1H), 4.48 (s, 1H), 4.35 (sxt, J=8.0 Hz, 1H), 3.75 (quin, J=8.5 Hz, 1H), 2.84-2.91 (m, 1H), 2.52-2.68 (m, 3H), 2.33-2.49 (m, 3H), 2.08-2.28 (m, 2H), 1.06-1.17 (m, 1H), 0.91-1.02 (m, 3H).

### Example 87-a, Example 87-b

### (Rₐ)—N-[6-(5-chloro-1,3-benzoxazol-2-yl)spiro[3.3]heptan-2-yl]-2-methylsulfonyl-pyridine-4-carboxamide

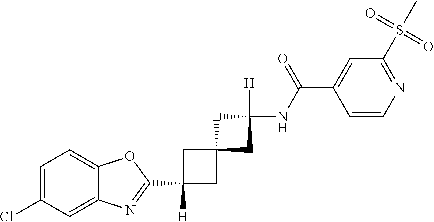

### (Sₐ)—N-[6-(5-chloro-1,3-benzoxazol-2-yl)spiro[3.3]heptan-2-yl]-2-methylsulfonyl-pyridine-4-carboxamide

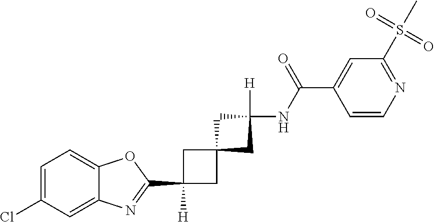

The two enantiomers (Example 84-a, Example 84-b) were obtained through SFC [Instrument: Shimadzu LC-20AT, Column: CHIRALPAK IQ 5.0 cm I.D.×25 cmL, 10 μm; Mobile phase: MeOH=100%; Flow rate: 60 mL/min; Wave length: UV 254 nm; Column temperature: 35° C.; elution order was Example 87-a, Example 87-b] chiral separation of N-[6-(5-chloro-1,3-benzoxazol-2-yl)spiro[3.3]heptan-2-yl]-2-methylsulfonyl-pyridine-4-carboxamide (Example 23).

Example 87-a: MS obsd. (ESI⁺) [(M+H)⁺]: 446.0. ¹H NMR (DMSO-d₆, 400 MHz) δ: 9.23 (d, J=7.2 Hz, 1H), 8.94 (d, J=4.9 Hz, 1H), 8.43 (s, 1H), 8.11 (dd, J=4.9, 1.5 Hz, 1H), 7.81 (d, J=2.1 Hz, 1H), 7.72 (d, J=8.7 Hz, 1H), 7.40 (dd, J=8.7, 2.1 Hz, 1H), 4.29-4.43 (m, 1H), 3.71-3.80 (m, 1H), 3.32 (s, 3H), 2.52-2.69 (m, 3H), 2.32-2.48 (m, 3H), 2.13-2.29 (m, 2H).

Example 87-b: MS obsd. (ESI⁺) [(M+H)⁺]: 446.1. ¹H NMR (DMSO-d₆, 400 MHz) δ: 9.22 (d, J=7.1 Hz, 1H), 8.91-8.97 (m, 1H), 8.40-8.47 (m, 1H), 8.11 (dd, J=4.9, 1.6 Hz, 1H), 7.80 (d, J=2.1 Hz, 1H), 7.72 (d, J=8.7 Hz, 1H), 7.40 (dd, J=8.7, 2.1 Hz, 1H), 4.36 (sxt, J=8.0 Hz, 1H), 3.76 (quin, J=8.5 Hz, 1H), 3.33 (s, 3H), 2.54-2.66 (m, 3H), 2.32-2.48 (m, 3H), 2.08-2.28 (m, 2H).

### Example 88

### N-[6-(6-chlorooxazolo[5,4-b]pyridin-2-yl)spiro[3.3]heptan-2-yl]-2-methylsulfonyl-pyridine-4-carboxamide

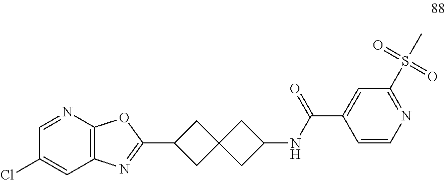 [SMILES: CS(=O)(=O)c1cc(C(=O)NC2CC3(C2)CC(c2nc4cc(Cl)cnc4o2)C3)ccn1]

The title compound was prepared in analogy to the procedure described for the preparation of Example 83, by using 2-methylsulfanylpyridine-4-carboxylic acid (Int-29) instead of 2-cyclopropylsulfanylpyridine-4-carboxylic acid (Int-28) and 6-(6-chlorooxazolo[5,4-b]pyridin-2-yl)spiro[3.3]heptan-2-amine (Int-3) instead of 6-(5-chloro-1,3-benzoxazol-2-yl)spiro[3.3]heptan-2-amine (Int-1) to afford Example 88 as a white solid.

MS obsd. (ESI⁺) [(M+H)⁺]: 447.1. ¹H NMR (MeOH-d₄, 400 MHz) δ: 8.88 (dd, J=5.0, 0.8 Hz, 1H), 8.43 (dd, J=1.6, 0.8 Hz, 1H), 8.30 (d, J=2.3 Hz, 1H), 8.12 (d, J=2.3 Hz, 1H), 8.03 (dd, J=4.9, 1.6 Hz, 1H), 4.44 (t, J=7.8 Hz, 1H), 3.78 (dd, J=16.9, 8.4 Hz, 1H), 3.27 (s, 3H), 2.74-2.48 (m, 6H), 2.31-2.18 (m, 2H).

### Example 89

### N-[6-(5-chloro-1,3-benzoxazol-2-yl)spiro[3.3]heptan-2-yl]-2-(methylsulfonimidoyl)pyridine-4-carboxamide

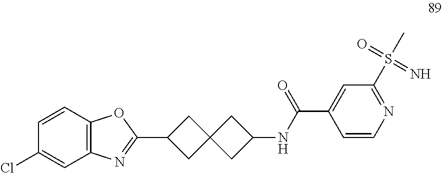 [SMILES: CS(=N)(=O)c1cc(C(=O)NC2CC3(C2)CC(c2nc4cc(Cl)ccc4o2)C3)ccn1]

The title compound was prepared in analogy to the procedure described for the preparation of Example 86, by using N-[6-(5-chloro-1,3-benzoxazol-2-yl)spiro[3.3]heptan-2-yl]-2-methylsulfanyl-pyridine-4-carboxamide instead of N-[6-(5-chloro-1,3-benzoxazol-2-yl)spiro[3.3]heptan-2-yl]-2-cyclopropylsulfanyl-pyridine-4-carboxamide to afford Example 89 as a pink solid.

MS obsd. (ESI⁺) [(M+H)⁺]: 445.1. ¹H NMR (DMSO-d₆, 400 MHz) δ: 9.16 (d, J=7.2 Hz, 1H), 8.86 (d, J=4.9 Hz, 1H), 8.44 (s, 1H), 7.99 (dd, J=4.9, 1.5 Hz, 1H), 7.81 (d, J=2.1 Hz, 1H), 7.72 (d, J 8.7 Hz, 1H), 7.40 (dd, J=8.7, 2.1 Hz, 1H), 4.52 (s, 1H), 4.30-4.44 (m, 1H), 3.76 (quin, J=8.5 Hz, 1H), 3.19 (s, 3H), 2.52-2.68 (m, 3H), 2.31-2.48 (m, 3H), 2.10-2.28 (m, 2H).

### Example 90

### 5-Chloro-N-[6-(5-chloro-1,3-benzoxazol-2-yl)spiro[3.3]heptan-2-yl]-2-methylsulfonyl-pyridine-4-carboxamide

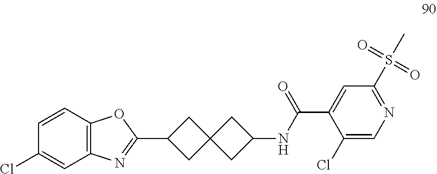 [SMILES: CS(=O)(=O)c1cc(C(=O)NC2CC3(C2)CC(c2nc4cc(Cl)ccc4o2)C3)c(Cl)cn1]

The title compound was prepared in analogy to the procedure described for the preparation of Example 83, by using 5-chloro-2-methylsulfanyl-pyridine-4-carboxylic acid (Int-30) instead of 2-cyclopropylsulfanylpyridine-4-carboxylic acid (Int-28) to afford Example 90 as a white solid.

MS obsd. (ESI⁺) [(M+H)⁺]: 480.0. ¹H NMR (DMSO-d₆, 400 MHz) δ: 9.07 (d, J=7.0 Hz, 1H), 8.96 (s, 1H), 8.05 (s, 1H), 7.80 (d, J=2.0 Hz, 1H), 7.72 (d, J=8.7 Hz, 1H), 7.40 (dd, J=8.6, 2.1 Hz, 1H), 4.21-4.32 (m, 1H), 3.69-3.79 (m, 1H), 3.33-3.34 (m, 3H), 2.53-2.64 (m, 3H), 2.35-2.47 (m, 3H), 2.00-2.20 (m, 2H).

### Example 91

### N-[6-(5-chloro-1,3-benzoxazol-2-yl)spiro[3.3]heptan-2-yl]-2-(1,1-dioxo-1,4-thiazinan-4-yl)pyridine-4-carboxamide

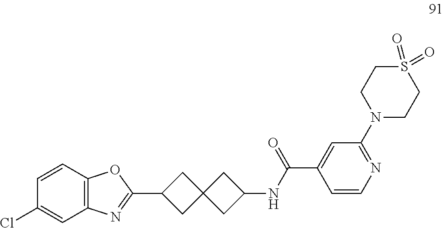 [SMILES: O=C(NC1CC2(C1)CC(c1nc3cc(Cl)ccc3o1)C2)c1ccnc(N2CCS(=O)(=O)CC2)c1]

The title compound was prepared in analogy to the procedure described for the preparation of Example 83, by using 2-thiomorpholinopyridine-4-carboxylic acid (Int-31) instead of 2-cyclopropylsulfanylpyridine-4-carboxylic acid (Int-28) to afford Example 91 as a white solid.

MS obsd. (ESI⁺) [(M+H)⁺]: 501.1. ¹H NMR (DMSO-d₆, 400 MHz) δ: 8.69 (br d, J=7.3 Hz, 1H), 8.24 (d, J=5.1 Hz, 1H), 7.80 (d, J=2.0 Hz, 1H), 7.72 (d, J=8.7 Hz, 1H), 7.40 (dd, J=8.7, 2.1 Hz, 1H), 7.28 (s, 1H), 7.06 (d, J=5.3 Hz, 1H), 4.26-4.39 (m, 1H), 4.10 (br s, 4H), 3.75 (quin, J=8.5 Hz, 1H), 3.08-3.19 (m, 4H), 2.54-2.65 (m, 3H), 2.46 (br d, J=8.3 Hz, 2H), 2.31-2.38 (m, 1H), 2.07-2.27 (m, 2H).

### Example 92 & Example 93

### N-[6-(5-chloro-1,3-benzoxazol-2-yl)spiro[3.3]heptan-2-yl]-N-methyl-2-methylsulfonyl-pyridine-4-carboxamide and N-[6-(5-chloro-1,3-benzoxazol-2-yl)spiro[3.3]heptan-2-yl]-2-ethylsulfonyl-N-methyl-pyridine-4-carboxamide

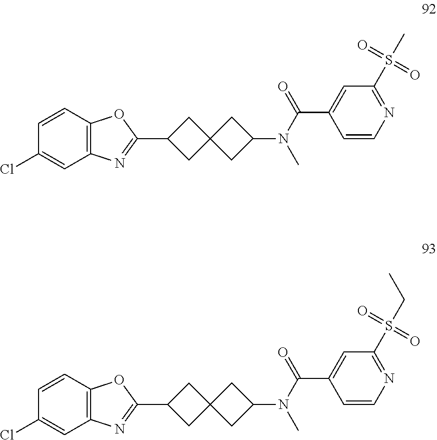 [SMILES: CN(C(=O)c1ccnc(S(C)(=O)=O)c1)C1CC2(CC(c3nc4cc(Cl)ccc4o3)C2)C1 ; CCS(=O)(=O)c1cc(C(=O)N(C)C2CC3(CC(c4nc5cc(Cl)ccc5o4)C3)C2)ccn1]

### Preparation of N-[6-(5-chloro-1,3-benzoxazol-2-yl)spiro[3.3]heptan-2-yl]-N-methyl-2-methylsulfonyl-pyridine-4-carboxamide and N-[6-(5-chloro-1,3-benzoxazol-2-yl)spiro[3.3]heptan-2-yl]-2-ethylsulfonyl-N-methyl-pyridine-4-carboxamide

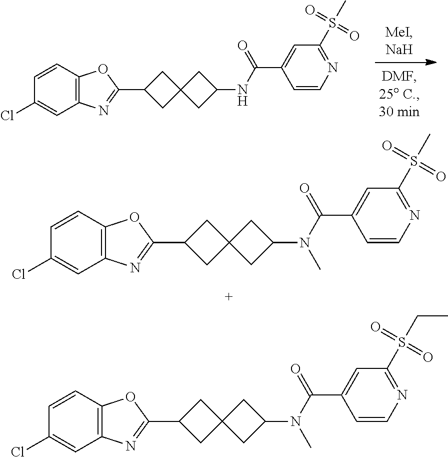 [SMILES: CS(=O)(=O)c1cc(C(=O)NC2CC3(C2)CC(c2nc4cc(Cl)ccc4o2)C3)ccn1 ; CN(C(=O)c1ccnc(S(C)(=O)=O)c1)C1CC2(CC(c3nc4cc(Cl)ccc4o3)C2)C1 ; CCS(=O)(=O)c1cc(C(=O)N(C)C2CC3(CC(c4nc5cc(Cl)ccc5o4)C3)C2)ccn1]

A mixture of N-[6-(5-chloro-1,3-benzoxazol-2-yl)spiro[3.3]heptan-2-yl]-2-methylsulfonyl-pyridine-4-carboxamide (Example 23) (330 mg, 0.74 mmol), NaH (53 mg, 2.22 mmol) and Mel (840 mg, 5.92 mmol) in DMF (14 mL) was stirred at 25° C. for 30 min. The reaction was quenched by water and the residue was extracted with EtOAc (50 mL×3). The organic layers were washed with brine (50 mL×3), dried over MgSO₄ and concentrated in vacuum. The residue was purified by pre-HPLC to give N-[6-(5-chloro-1,3-benzoxazol-2-yl)spiro[3.3]heptan-2-yl]-N-methyl-2-methylsulfonyl-pyridine-4-carboxamide (Example 92) (24.6 mg) and N-[6-(5-chloro-1,3-benzoxazol-2-yl)spiro[3.3]heptan-2-yl]-2-ethylsulfonyl-N-methyl-pyridine-4-carboxamide (Example 93) (96.3 mg) as a white solid.

MS obsd. (ESI⁺) [(M+H)⁺]: 460.1. ¹H NMR (DMSO-d₆, 400 MHz) δ: 8.87-8.88 (m, 1H), 7.94-8.01 (m, 1H), 7.66-7.82 (m, 3H), 7.40 (br t, J=7.0 Hz, 1H), 4.76 (br t, J=8.3 Hz, 0.4H), 3.98 (quin, J=8.1 Hz, 0.6H), 3.61-3.83 (m, 1H), 3.32-3.34 (m, 3H), 2.98 (s, 1.7H), 2.81 (s, 1.3H), 2.53-2.71 (m, 1H), 2.23-2.49 (m, 7H).

MS obsd. (ESI⁺) [(M+H)⁺]: 474.1. ¹H NMR (DMSO-d₆, 400 MHz) δ: 8.83-8.96 (m, 1H), 7.94-8.01 (m, 1H), 7.65-7.84 (m, 3H), 7.40 (br t, J=6.8 Hz, 1H), 4.67-4.86 (m, 0.4H), 3.95 (br t, J=8.4 Hz, 0.6H), 3.61-3.84 (m, 1H), 3.49 (q, J=7.5 Hz, 2H), 2.98 (s, 1.7H), 2.80 (s, 1.3H), 2.53-2.69 (m, 1H), 2.03-2.49 (m, 7H), 1.15 (t, J=7.3 Hz, 3H).

### Example 94-a, Example 94-b, Example 94-c & Example 94-d

### (Rₐ)-(3R)—N-[6-(5-chloro-1,3-benzoxazol-2-yl)spiro[3.3]heptan-2-yl]-1-methyl-5-oxo-pyrrolidine-3-carboxamide

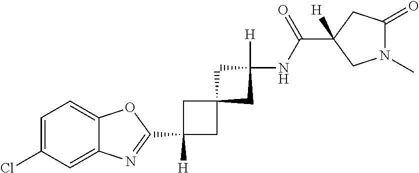

### (Rₐ)-(3S)—N-[6-(5-chloro-1,3-benzoxazol-2-yl)spiro[3.3]heptan-2-yl]-1-methyl-5-oxo-pyrrolidine-3-carboxamide

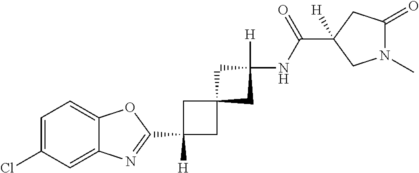

### (Sₐ)-(3R)—N-[6-(5-chloro-1,3-benzoxazol-2-yl)spiro[3.3]heptan-2-yl]-1-methyl-5-oxo-pyrrolidine-3-carboxamide

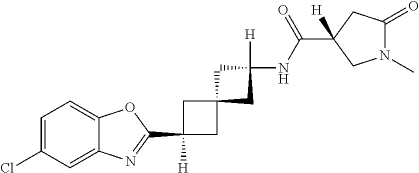

### (Sₐ)-(3S)—N-[6-(5-chloro-1,3-benzoxazol-2-yl)spiro[3.3]heptan-2-yl]-1-methyl-5-oxo-pyrrolidine-3-carboxamide

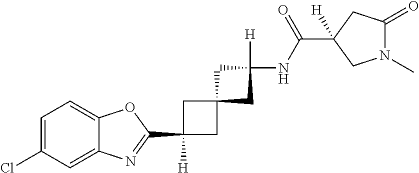

The four diastereomers (Example 94-a, Example 94-b, Example 94-c and Example 94-d) were obtained through SFC [Instrument: SFC 80, Column: AD, 250×20 mm I.D., 5 μm; Mobile phase: A for CO₂ and B for ethanol (0.25% DEA); Gradient: B 30%; Flow rate: 50 mL/min; Back pressure: 100 bar; Column temperature: 35° C.; elution order was Example 94-a, Example 94-b, Example 94-c and Example 94-d] chiral separation of N-[6-(5-chloro-1,3-benzoxazol-2-yl)spiro[3.3]heptan-2-yl]-1-methyl-5-oxo-pyrrolidine-3-carboxamide (Example 30).

Example 94-a: MS obsd. (ESI⁺) [(M+H)⁺]: 388.1. ¹H NMR (DMSO-d₆, 400 MHz) δ: 8.24 (d, J=7.3 Hz, 1H), 7.79 (d, J=2.1 Hz, 1H), 7.71 (d, J=8.7 Hz, 1H), 7.40 (dd, J=8.7, 2.1 Hz, 1H), 4.08 (sxt, J=8.0 Hz, 1H), 3.71 (quin, J=8.5 Hz, 1H), 3.36-3.53 (m, 1H), 3.27-3.31 (m, 1H), 2.93-3.09 (m, 1H), 2.68 (s, 3H), 2.51-2.59 (m, 2H), 2.44-2.48 (m, 1H), 2.41 (br dd, J=8.5, 4.8 Hz, 2H), 2.31-2.38 (m, 2H), 2.27 (ddd, J=11.7, 7.2, 4.8 Hz, 1H), 1.95-2.06 (m, 1H), 1.91 (dd, J=11.1, 8.8 Hz, 1H).

Example 94-b: MS obsd. (ESI⁺) [(M+H)⁺]: 388.1. ¹H NMR (DMSO-d₆, 400 MHz) δ: 8.25 (d, J=7.3 Hz, 1H), 7.79 (d, J=2.0 Hz, 1H), 7.71 (d, J=8.6 Hz, 1H), 7.40 (dd, J=8.7, 2.1 Hz, 1H), 4.09 (sxt, J=8.0 Hz, 1H), 3.72 (quin, J=8.4 Hz, 1H), 3.46 (t, J=9.3 Hz, 1H), 3.29 (dd, J=9.6, 6.2 Hz, 1H), 2.96-3.07 (m, 1H), 2.68 (s, 3H), 2.51-2.60 (m, 2H), 2.44-2.48 (m, 1H), 2.32-2.43 (m, 4H), 2.24-2.31 (m, 1H), 2.00 (dd, J=10.7, 8.9 Hz, 1H), 1.91 (dd, J=11.2, 8.8 Hz, 1H).

Example 94-c: MS obsd. (ESI⁺) [(M+H)⁺]: 388.1. ¹H NMR (DMSO-d₆, 400 MHz) δ: 8.24 (d, J=7.3 Hz, 1H), 7.79 (d, J=2.1 Hz, 1H), 7.71 (d, J=8.7 Hz, 1H), 7.40 (dd, J=8.7, 2.2 Hz, 1H), 4.08 (sxt, J=8.0 Hz, 1H), 3.72 (quin, J=8.5 Hz, 1H), 3.48-3.51 (m, 1H), 3.42-3.45 (m, 1H), 2.85-3.07 (m, 1H), 2.69 (s, 3H), 2.51-2.60 (m, 2H), 2.44-2.48 (m, 1H), 2.38-2.44 (m, 2H), 2.31-2.38 (m, 2H), 2.24-2.31 (m, 1H), 2.00 (dd, J=10.7, 8.9 Hz, 1H), 1.91 (dd, J=11.2, 8.7 Hz, 1H).

Example 94-d: MS obsd. (ESI⁺) [(M+H)⁺]: 388.1. ¹H NMR (DMSO-d₆, 400 MHz) δ: 8.25 (d, J=7.3 Hz, 1H), 7.79 (d, J=2.1 Hz, 1H), 7.71 (d, J=8.7 Hz, 1H), 7.40 (dd, J=8.7, 2.1 Hz, 1H), 4.08 (sxt, J=7.9 Hz, 1H), 3.72 (quin, J=8.5 Hz, 1H), 3.41-3.51 (m, 1H), 3.29 (dd, J=9.7, 6.2 Hz, 1H), 2.95-3.08 (m, 1H), 2.68 (s, 3H), 2.52-2.60 (m, 2H), 2.44-2.48 (m, 1H), 2.31-2.44 (m, 4H), 2.24-2.31 (m, 1H), 2.00 (dd, J=10.6, 8.9 Hz, 1H), 1.91 (dd, J=11.2, 8.8 Hz, 1H).

### Example 95

### N-[6-(5-chloro-1,3-benzoxazol-2-yl)spiro[3.3]heptan-2-yl]-2-(N-hydroxycarbamimidoyl)pyridine-4-carboxamide

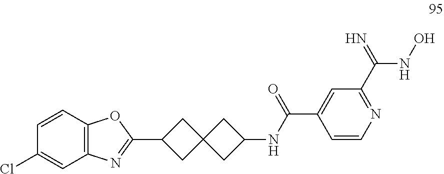 [SMILES: N=C(NO)c1cc(C(=O)NC2CC3(C2)CC(c2nc4cc(Cl)ccc4o2)C3)ccn1]

### Preparation of N-[6-(5-chloro-1,3-benzoxazol-2-yl)spiro[3.3]heptan-2-yl]-2-cyano-pyridine-4-carboxamide

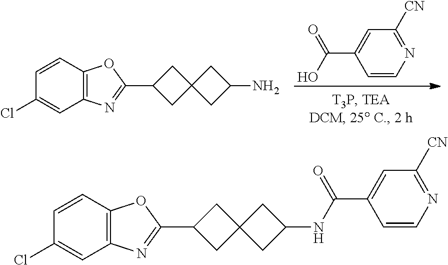 [SMILES: NC1CC2(C1)CC(c1nc3cc(Cl)ccc3o1)C2 ; N#Cc1cc(C(=O)O)ccn1 ; N#Cc1cc(C(=O)NC2CC3(C2)CC(c2nc4cc(Cl)ccc4o2)C3)ccn1]

To a solution of 6-(5-chloro-1,3-benzoxazol-2-yl)spiro[3.3]heptan-2-amine (Int-1) (200 mg, 0.76 mmol) and 2-cyanopyridine-4-carboxylic acid (112 mg, 0.76 mmol) in DCM (10 mL) was added T₃P (1453 mg, 2.28 mmol) and TEA (0.32 mL, 2.28 mmol). The reaction mixture was stirred at 25° C. for 2 h. Then the reaction was diluted with DCM (30 ml) and water (20 mL). The organic layer was separated and washed with brine, dried over Na₂SO₄. The solvent was concentrated to dryness and the residue was purified by silica gel column eluted with DCM/MeOH=15/1 to give N-[6-(5-chloro-1,3-benzoxazol-2-yl)spiro[3.3]heptan-2-yl]-2-cyano-pyridine-4-carboxamide (134 mg) as a white powder.

MS obsd. (ESI⁺) [(M+H)⁺]: 393.1.

### Preparation of N-[6-(5-chloro-1,3-benzoxazol-2-yl)spiro[3.3]heptan-2-yl]-2-(N-hydroxycarbamimidoyl)pyridine-4-carboxamide

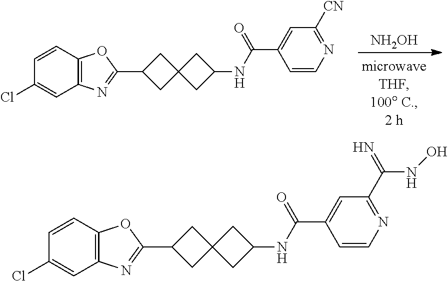

To a 20 mL microwave vial was added hydroxylamine (33.6 mg, 509 μmol, 50% in water) and N-(6-(5-chlorobenzo[d]oxazol-2-yl)spiro[3.3]heptan-2-yl)-2-cyanoisonicotinamide (100 mg, 255 μmol) in THF (3 ml). The vial was capped and heated under microwave at 100° C. for 2 h. The reaction solution was concentrated in vacuum to give N-[6-(5-chloro-1,3-benzoxazol-2-yl)spiro[3.3]heptan-2-yl]-2-(N-hydroxycarbamimidoyl)pyridine-4-carboxamide (105 mg) as an off-white solid.

MS obsd. (ESI⁺) [(M+H)⁺]: 426.3. ¹H NMR (DMSO-d₆, 400 MHz) δ: 9.98 (s, 1H), 9.00 (s, 1H), 8.64-8.70 (m, 1H), 8.21 (s, 1H), 7.80 (d, J=2.0 Hz, 1H), 7.69-7.77 (m, 2H), 7.40 (dd, J=8.7, 2.2 Hz, 1H), 4.25-4.39 (m, 1H), 3.75 (s, 1H), 2.52-2.67 (m, 3H), 2.31-2.48 (m, 3H), 1.92-2.26 (m, 2H).

### Example 96

### 2-Carbamimidoyl-N-[6-(5-chloro-1,3-benzoxazol-2-yl)spiro[3.3]heptan-2-yl]pyridine-4-carboxamide

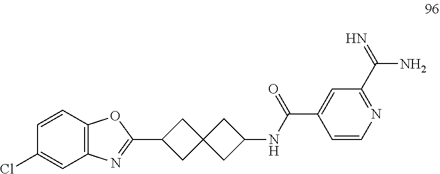 [SMILES: N=C(N)c1cc(C(=O)NC2CC3(C2)CC(c2nc4cc(Cl)ccc4o2)C3)ccn1]

### Preparation of 2-carbamimidoyl-N-[6-(5-chloro-1,3-benzoxazol-2-yl)spiro[3.3]heptan-2-yl]pyridine-4-carboxamide

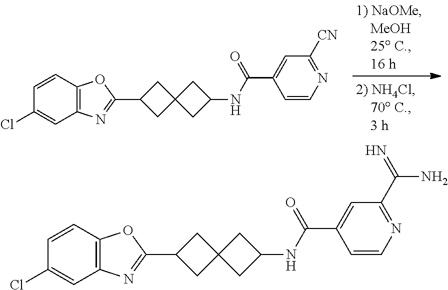 [SMILES: N#Cc1cc(C(=O)NC2CC3(C2)CC(c2nc4cc(Cl)ccc4o2)C3)ccn1 ; N=C(N)c1cc(C(=O)NC2CC3(C2)CC(c2nc4cc(Cl)ccc4o2)C3)ccn1]

To a solution of N-[6-(5-chloro-1,3-benzoxazol-2-yl)spiro[3.3]heptan-2-yl]-2-cyano-pyridine-4-carboxamide (134 mg, 0.34 mmol) in methanol (3 mL) was added sodium methanolate (18 mg, 0.34 mmol). The reaction mixture was stirred at room temperature for 16 h. Then ammonium chloride (22 mg, 0.410 mmol) was added and the reaction was heated to 70° C. for 3 h. After cooing to room temperature, the reaction was concentrated to dryness and the residue was purified by silica gel column eluted with DCM/MeOH=10/1 to give 2-carbamimidoyl-N-[6-(5-chloro-1,3-benzoxazol-2-yl)spiro[3.3]heptan-2-yl]pyridine-4-carboxamide (46 mg) as a white solid.

MS obsd. (ESI⁺) [(M+H)⁺]: 410.1. ¹H NMR (CDCl₃, 400 MHz) δ: 9.76 (br s, 2H), 9.56 (s, 1H), 9.15 (br s, 1H), 8.85 (d, J=4.9 Hz, 1H), 8.44 (br d, J=6.8 Hz, 1H), 8.24-8.31 (m, 1H), 7.64 (d, J 1.7 Hz, 1H), 7.39 (d, J=8.7 Hz, 1H), 7.27-7.31 (m, 1H), 4.48-4.59 (m, 1H), 3.67 (quin, J=8.5 Hz, 1H), 2.45-2.74 (m, 6H), 2.33-2.43 (m, 2H).

### Example 97

### N-[6-(5-chloro-1,3-benzoxazol-2-yl)spiro[3.3]heptan-2-yl]-2-(methylamino)pyridine-4-carboxamide

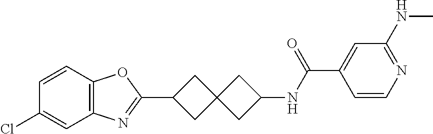 [SMILES: CNc1cc(C(=O)NC2CC3(C2)CC(c2nc4cc(Cl)ccc4o2)C3)ccn1]

The title compound was prepared in analogy to the procedure described for the preparation of Example 95, by using 2-chloropyridine-4-carboxylic acid instead of 2-cyanopyridine-4-carboxylic acid and methylamine instead of hydroxylamine to afford Example 97 as a light yellow solid.

MS obsd. (ESI⁺) [(M+H)⁺]: 397.2. ¹H NMR (DMSO-d₆, 400 MHz) δ: 8.62 (br d, J=7.5 Hz, 1H), 8.05 (d, J=5.3 Hz, 1H), 7.80 (d, J=2.0 Hz, 1H), 7.72 (d, J=8.7 Hz, 1H), 7.40 (dd, J=8.7, 2.1 Hz, 1H), 6.81 (dd, J=5.3, 1.1 Hz, 1H), 6.78 (s, 1H), 6.65 (br d, J=4.8 Hz, 1H), 4.29 (sxt, J=8.0 Hz, 1H), 3.74 (quin, J=8.4 Hz, 1H), 2.78 (d, J=4.8 Hz, 3H), 2.53-2.69 (m, 3H), 2.38-2.47 (m, 2H), 2.26-2.37 (m, 1H), 2.07-2.24 (m, 2H).

### Example 98

### 2-(Azetidin-3-ylsulfonyl)-N-[6-(5-chloro-1,3-benzoxazol-2-yl)spiro[3.3]heptan-2-yl]pyridine-4-carboxamide

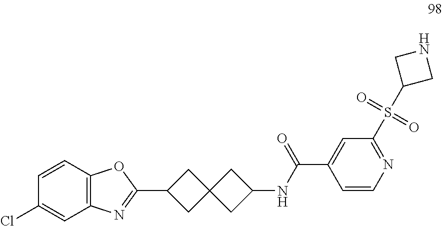 [SMILES: O=C(NC1CC2(C1)CC(c1nc3cc(Cl)ccc3o1)C2)c1ccnc(S(=O)(=O)C2CNC2)c1]

### Preparation of tert-butyl 3-[[4-[[6-(5-chloro-1,3-benzoxazol-2-yl)spiro[3.3]heptan-2-yl]carbamoyl]-2-pyridyl]sulfonyl]azetidine-1-carboxylate

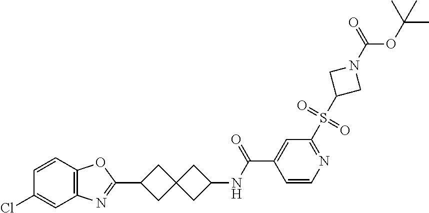 [SMILES: CC(C)(C)OC(=O)N1CC(S(=O)(=O)c2cc(C(=O)NC3CC4(C3)CC(c3nc5cc(Cl)ccc5o3)C4)ccn2)C1]

The title compound was prepared in analogy to the procedure described for the preparation of Example 1, by using 2-(1-tert-butoxycarbonylazetidin-3-yl)sulfonylpyridine-4-carboxylic acid (Int-32) instead of pyridine-2-carboxylic acid to afford tert-butyl 3-[[4-[[6-(5-chloro-1,3-benzoxazol-2-yl)spiro[3.3]heptan-2-yl]carbamoyl]-2-pyridyl]sulfonyl]azetidine-1-carboxylate as a yellow solid.

MS obsd. (ESI⁺) [(M+H)⁺]: 587.1

### Preparation of 2-(azetidin-3-ylsulfonyl)-N-[6-(5-chloro-1,3-benzoxazol-2-yl)spiro[3.3]heptan-2-yl]pyridine-4-carboxamide

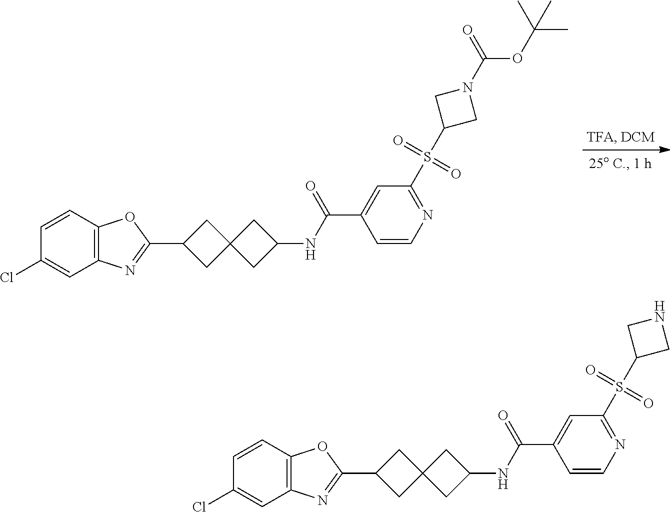 [SMILES: CC(C)(C)OC(=O)N1CC(S(=O)(=O)c2cc(C(=O)NC3CC4(C3)CC(c3nc5cc(Cl)ccc5o3)C4)ccn2)C1 ; O=C(NC1CC2(C1)CC(c1nc3cc(Cl)ccc3o1)C2)c1ccnc(S(=O)(=O)C2CNC2)c1]

To a solution of tert-butyl 3-[[4-[[6-(5-chloro-1,3-benzoxazol-2-yl)spiro[3.3]heptan-2-yl]carbamoyl]-2-pyridyl]sulfonyl]azetidine-1-carboxylate (100 mg, 0.17 mmol) was added TFA (0.01 mL, 0.18 mmol). Then the reaction was stirred at 25° C. for 1 h. The mixture was diluted with DCM (30 mL×3) and washed with saturated aqueous NaHCO₃ (10 mL×2). The organic layers were dried over Na₂SO₄ and concentrated. The residue was purified by prep-HPLC to give 2-(azetidin-3-ylsulfonyl)-N-[6-(5-chloro-1,3-benzoxazol-2-yl)spiro[3.3]heptan-2-yl]pyridine-4-carboxamide (45 mg) as a white solid.

MS obsd. (ESI⁺) [(M+H)⁺]: 487.1. ¹H NMR (DMSO-d₆, 400 MHz) δ: 9.23 (br d, J=7.1 Hz, 1H), 8.91 (d, J=4.9 Hz, 1H), 8.44 (s, 1H), 8.10 (d, J=4.9 Hz, 1H), 7.81 (d, J=2.0 Hz, 1H), 7.72 (d, J=8.7 Hz, 1H), 7.40 (dd, J=8.6, 1.9 Hz, 1H), 4.71 (quin, J=7.5 Hz, 1H), 4.35 (sxt, J=7.9 Hz, 1H), 3.71-3.88 (m, 3H), 3.61 (t, J=8.7 Hz, 2H), 2.53-2.68 (m, 3H), 2.33-2.48 (m, 3H), 2.12-2.28 (m, 2H).

### Example 99

### 2-(Acetylsulfamoyl)-N-[6-(5-chloro-1,3-benzoxazol-2-yl)spiro[3.3]heptan-2-yl]pyridine-4-carboxamide

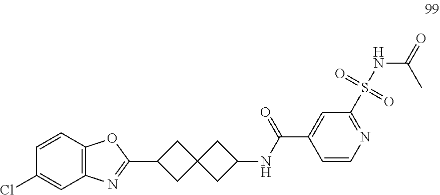 [SMILES: CC(=O)NS(=O)(=O)c1cc(C(=O)NC2CC3(C2)CC(c2nc4cc(Cl)ccc4o2)C3)ccn1]

### Preparation of 2-(acetylsulfamoyl)-N-[6-(5-chloro-1,3-benzoxazol-2-yl)spiro[3.3]heptan-2-yl]pyridine-4-carboxamide

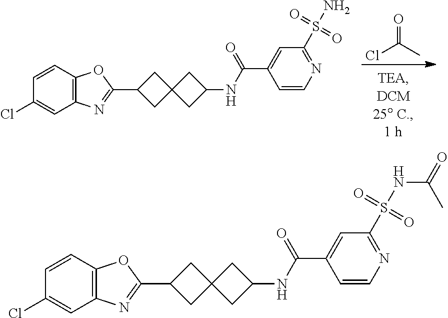

To a solution of N-[6-(5-chloro-1,3-benzoxazol-2-yl)spiro[3.3]heptan-2-yl]-2-sulfamoyl-pyridine-4-carboxamide (27.0 mg, 0.060 mmol) and triethylamine (0.03 mL, 0.18 mmol) in DCM (4 mL) was added acetyl chloride (0.01 mL, 0.070 mmol). Then the reaction was stirred at 25° C. for 1 h. The mixture was diluted with EtOAc (30 mL×3) and washed with water (20 mL×2). The organic layers were dried over Na₂SO₄ and concentrated. The residue was purified by prep-HPLC to give 2-(acetylsulfamoyl)-N-[6-(5-chloro-1,3-benzoxazol-2-yl)spiro[3.3]heptan-2-yl]pyridine-4-carboxamide (6.1 mg) as a white solid.

MS obsd. (ESI⁺) [(M+H)⁺]: 489.1. ¹H NMR (DMSO-d₆, 400 MHz) δ: 11.94-12.79 (br, 1H), 9.14 (d, J=7.3 Hz, 1H), 8.73 (d, J=5.0 Hz, 1H), 8.30 (s, 1H), 7.88 (d, J=4.9 Hz, 1H), 7.81 (d, J=2.0 Hz, 1H), 7.69-7.75 (m, 1H), 7.40 (dd, J=8.7, 2.2 Hz, 1H), 4.35 (sxt, J=8.0 Hz, 1H), 3.75 (quin, J=8.5 Hz, 1H), 2.52-2.68 (m, 3H), 2.32-2.48 (m, 3H), 2.13-2.28 (m, 2H), 1.78 (s, 3H).

### Example 100

### N-[6-(5-chloro-1,3-benzoxazol-2-yl)spiro[3.3]heptan-2-yl]-2-(cyclopropylmethylsulfonyl)pyridine-4-carboxamide

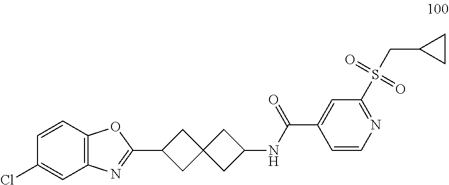 [SMILES: O=C(NC1CC2(C1)CC(c1nc3cc(Cl)ccc3o1)C2)c1ccnc(S(=O)(=O)CC2CC2)c1]

### Preparation of 2-bromo-N-[6-(5-chloro-1,3-benzoxazol-2-yl)spiro[3.3]heptan-2-yl]pyridine-4-carboxamide

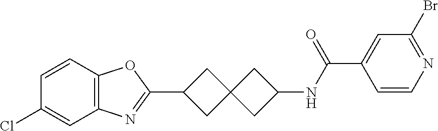 [SMILES: O=C(NC1CC2(C1)CC(c1nc3cc(Cl)ccc3o1)C2)c1ccnc(Br)c1]

The title compound was prepared in analogy to the procedure described for the preparation of Example 1, by using 2-bromopyridine-4-carboxylic acid instead of pyridine-2-carboxylic acid to afford 2-bromo-N-[6-(5-chloro-1,3-benzoxazol-2-yl)spiro[3.3]heptan-2-yl]pyridine-4-carboxamide as a white solid.

MS obsd. (ESI⁺) [(M+H)⁺]: 446.0

### Preparation of N-[6-(5-chloro-1,3-benzoxazol-2-yl)spiro[3.3]heptan-2-yl]-2-(cyclopropylmethylsulfonyl)pyridine-4-carboxamide

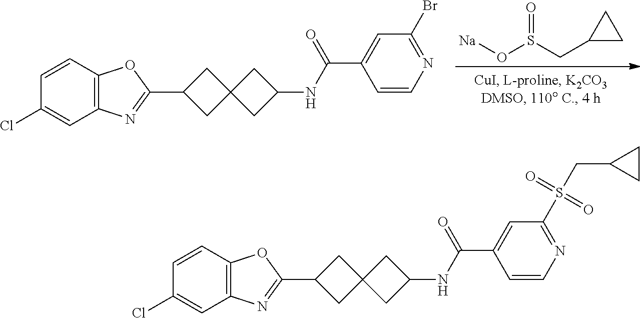

To a 50 mL of three necked flash was added 2-bromo-N-[6-(5-chloro-1,3-benzoxazol-2-yl)spiro[3.3]heptan-2-yl]pyridine-4-carboxamide (100 mg, 0.22 mmol), cyclopropylmethylsulfinyloxysodium (38 mg, 0.27 mmol), cuprous iodide (42 mg, 0.22 mmol), L-proline (25 mg, 0.22 mmol) and potassium carbonate (31 mg, 0.22 mmol). DMSO (5 mL) was added by syringe and the reaction was bubbled with N₂. Then the reaction was stirred at 110° C. for 4 h. After cooling to room temperature, 1 N HCl (20 mL) was added and extracted with DCM (20 mL×3). The combined organic phase was washed with water (50 mL), brine (50 mL) and dried over Na₂SO₄. The solvent was concentrated to dryness and the residue was purified by prep-HPLC to give N-[6-(5-chloro-1,3-benzoxazol-2-yl)spiro[3.3]heptan-2-yl]-2-(cyclopropylmethylsulfonyl)pyridine-4-carboxamide (Example 100) (21 mg) as a white solid.

MS obsd. (ESI⁺) [(M+H)⁺]: 486.1. ¹H NMR (DMSO-d₆, 400 MHz) δ: 8.87 (br d, J=4.4 Hz, 1H), 8.31 (s, 1H), 7.99 (br d, J=3.9 Hz, 1H), 7.66 (br s, 1H), 7.41 (br d, J=8.7 Hz, 1H), 7.28-7.31 (m, 1H), 6.61 (br d, J=6.4 Hz, 1H), 4.54 (sxt, J=7.9 Hz, 1H), 3.71 (br t, J=8.4 Hz, 1H), 3.37 (d, J=7.2 Hz, 2H), 2.70-2.81 (m, 1H), 2.46-2.70 (m, 5H), 2.06-2.25 (m, 2H), 0.97-1.10 (m, 1H), 0.49-0.59 (m, 2H), 0.14-0.24 (m, 2H).

### Example 101

### N4-[6-(5-chloro-1,3-benzoxazol-2-yl)spiro[3.3]heptan-2-yl]pyridine-2,4-dicarboxamide

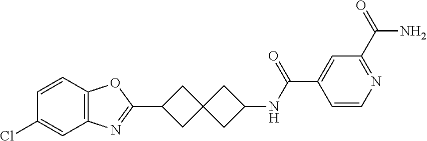 [SMILES: NC(=O)c1cc(C(=O)NC2CC3(C2)CC(c2nc4cc(Cl)ccc4o2)C3)ccn1]

### Preparation of methyl 4-[[6-(5-chloro-1,3-benzoxazol-2-yl)spiro[3.3]heptan-2-yl]carbamoyl]pyridine-2-carboxylate

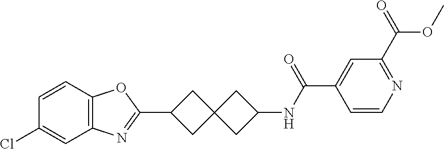 [SMILES: COC(=O)c1cc(C(=O)NC2CC3(C2)CC(c2nc4cc(Cl)ccc4o2)C3)ccn1]

The title compound was prepared in analogy to the procedure described for the preparation of Example 1, by using 2-methoxycarbonylpyridine-4-carboxylic acid instead of pyridine-2-carboxylic acid to afford methyl 4-[[6-(5-chloro-1,3-benzoxazol-2-yl)spiro[3.3]heptan-2-yl]carbamoyl]pyridine-2-carboxylate as colorless oil.

MS obsd. (ESI⁺) [(M+H)⁺]: 426.0

### Preparation of 4-[[6-(5-chloro-1,3-benzoxazol-2-yl)spiro[3.3]heptan-2-yl]carbamoyl]pyridine-2-carboxylic acid

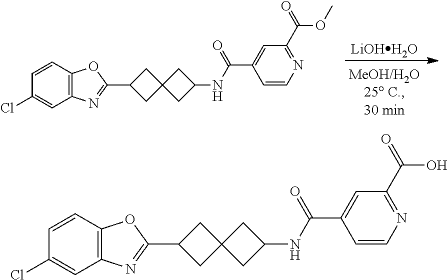

To a solution of methyl 4-[[6-(5-chloro-1,3-benzoxazol-2-yl)spiro[3.3]heptan-2-yl]carbamoyl]pyridine-2-carboxylate (1.2 g, 2.82 mmol) in methanol (15 mL) was added a 2 M LiOH solution (5.0 mL, 2.82 mmol), then the reaction was stirred at 25° C. for 30 min. The reaction mixture was concentrated to remove MeOH. The aqueous solution was acidified with 2 N HCl solution to adjust pH=4, extracted with EtOAc (50 mL×2). The organic layers were concentrated to give 4-[[6-(5-chloro-1,3-benzoxazol-2-yl)spiro[3.3]heptan-2-yl]carbamoyl]pyridine-2-carboxylic acid (1 g) as colorless oil.

MS obsd. (ESI⁺) [(M+H)⁺]: 412.0.

### Preparation of N4-[6-(5-chloro-1,3-benzoxazol-2-yl)spiro[3.3]heptan-2-yl]pyridine-2,4-dicarboxamide

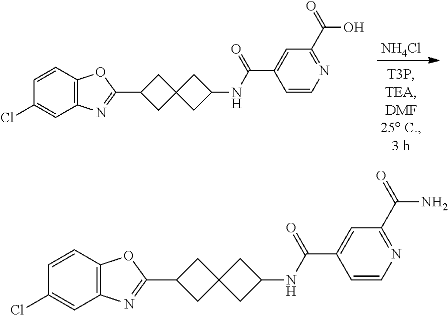 [SMILES: O=C(NC1CC2(C1)CC(c1nc3cc(Cl)ccc3o1)C2)c1ccnc(C(=O)O)c1 ; "NH4Cl" ; NC(=O)c1cc(C(=O)NC2CC3(C2)CC(c2nc4cc(Cl)ccc4o2)C3)ccn1]

To a solution of T₃P (224 mg, 0.70 mmol) in DMF (5 mL) was added 4-[[6-(5-chloro-1,3-benzoxazol-2-yl)spiro[3.3]heptan-2-yl]carbamoyl]pyridine-2-carboxylic acid (96 mg, 0.23 mmol). There after TEA (0.1 mL, 0.70 mmol) and ammonium chloride (37 mg, 0.70 mmol) was added and the mixture was stirred at 25° C. for 3 h. The reaction was diluted with DCM (20 ml) and washed with water (10 ml×2). The organic layer was dried over Na₂SO₄ and concentrated in vacuum. The residue was purified by pre-HPLC to give N4-[6-(5-chloro-1,3-benzoxazol-2-yl)spiro[3.3]heptan-2-yl]pyridine-2,4-dicarboxamide (36 mg) as a white solid.

MS obsd. (ESI⁺) [(M+H)⁺]: 411.1. ¹H NMR (DMSO-d₆, 400 MHz) δ: 9.12 (br d, J=7.3 Hz, 1H), 8.77 (d, J=5.0 Hz, 1H), 8.46 (s, 1H), 8.21 (br s, 1H), 7.94 (d, J=5.3 Hz, 1H), 7.66-7.82 (m, 3H), 7.40 (dd, J=8.6, 2.0 Hz, 1H), 4.27-4.41 (m, 1H), 3.75 (quin, J=8.5 Hz, 1H), 2.55-2.68 (m, 3H), 2.31-2.48 (m, 3H), 2.12-2.27 (m, 2H).

### Example 102

### N4-[6-(5-chloro-1,3-benzoxazol-2-yl)spiro[3.3]heptan-2-yl]-N2-methyl-pyridine-2,4-dicarboxamide

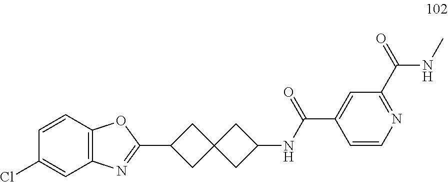 [SMILES: CNC(=O)c1cc(C(=O)NC2CC3(C2)CC(c2nc4cc(Cl)ccc4o2)C3)ccn1]

The title compound was prepared in analogy to the procedure described for the preparation of Example 101, by using methanamine instead of ammonium chloride to afford Example 102 as a white solid.

MS obsd. (ESI⁺) [(M+H)⁺]: 425.1. ¹H NMR (CDCl₃, 400 MHz) δ: 8.68 (d, J=4.6 Hz, 1H), 8.36 (s, 1H), 8.05 (br d, J=4.4 Hz, 1H), 7.94 (dd, J=4.9, 1.7 Hz, 1H), 7.65 (d, J=2.0 Hz, 1H), 7.40 (d, J=8.6 Hz, 1H), 7.29 (s, 1H), 6.62 (br d, J=7.3 Hz, 1H), 4.54 (sxt, J=8.0 Hz, 1H), 3.71 (quin, J=8.6 Hz, 1H), 3.07 (d, J=5.1 Hz, 3H), 2.71-2.81 (m, 1H), 2.55-2.67 (m, 4H), 2.44-2.53 (m, 1H), 1.99-2.24 (m, 2H).

### Example 103

### N4-[6-(5-chloro-1,3-benzoxazol-2-yl)spiro[3.3]heptan-2-yl]-N2,N2-dimethyl-pyridine-2,4-dicarboxamide

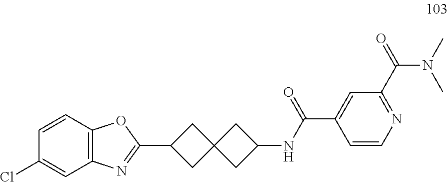 [SMILES: CN(C)C(=O)c1cc(C(=O)NC2CC3(C2)CC(c2nc4cc(Cl)ccc4o2)C3)ccn1]

The title compound was prepared in analogy to the procedure described for the preparation of Example 101, by using N-methylmethanamine hydrochloride instead of ammonium chloride to afford Example 103 as a white solid.

MS obsd. (ESI⁺) [(M+H)⁺]: 439.1. ¹H NMR (DMSO-d₆, 400 MHz) δ: 8.99 (d, J=7.2 Hz, 1H), 8.72 (d, J=5.0 Hz, 1H), 7.94 (s, 1H), 7.79-7.83 (m, 2H), 7.72 (d, J=8.7 Hz, 1H), 7.40 (dd, J=8.7, 2.1 Hz, 1H), 4.34 (sxt, J=7.9 Hz, 1H), 3.75 (quin, J=8.5 Hz, 1H), 3.03 (s, 3H), 2.93 (s, 3H), 2.53-2.68 (m, 3H), 2.42-2.48 (m, 2H), 2.31-2.38 (m, 1H), 2.07-2.26 (m, 2H).

### Example 104

### PHH Natural Infection Assay

Detailed procedures regarding primary human hepatocyte (PHH) HBV natural infection assay are described as below. One tube of frozen PHH (10 million cells) is thawed in 37° C. water bath and then transferred to 20 mL of PHH thawing medium (Sigma, InVitroGRO HT Medium, Cat. S03319) with gently mixing. The cells were then centrifuged at 80 g/min for 5 min, the supernatant was discarded and the tube was refilled with 25 mL of PHH plating medium (Sigma, InVitroGRO CP Medium, Cat. S03317). The tube was shaken very gently to re-suspend all cells, and then 50 μL of cells were transferred to each well 384-well collagen I coated plate with appropriate liquid handling equipment, e.g. Integra VIAFLO384 or Agilent Bravo. The cells were then cultured for 24 hours in a cell incubator. For HBV infection, after PHH attachment on the culture plate, the plating medium was removed and replenished with PHH culture medium containing HBV virus. The PHH culture medium was prepared with Dulbecco's Modified Eagle Medium (DMEM)/F12 (1:1 in volume ratio) containing 10% fetal bovine serum (Gibco, Cat. 10099141), 5 ng/mL human epidermal growth factor (Gibco, Cat.PHG0311L), 20 ng/mL dexamethasone (Sigma, Cat.D4902-100 mg), 250 ng/mL human recombinant insulin (Gibco, Cat.41400045) and 100 U/mL penicillin. HBV virus at 200 genome equivalent (GE) per cell with 4% PEG8000 (Sigma, Cat.P1458) containing culture medium were added to the PHH culture medium for infection. The cells were then cultured for 24 hours in cell incubator. Then the cell culture supernatant was removed. The HBV-infected PHH were cultured with sandwich culture method with PHH culture medium containing 10 DMSO and 0.25 mg/mL matrix gel for 72 hours. The supernatant was then refreshed with PHH culture medium containing different concentrations of testing compounds for two times with 72-hour interval. At the end of treatment, the supernatant was collected for viral markers measurements, including HBsAg, HBeAg, HBV DNA and cytotoxicity. HBsAg and HBeAg were detected using alphalisa method using their specific antibodies. For HBV DNA detection, HBV DNA Quantitative Fluorescence Diagnostic Kit (Sansure Biotech Inc.) was used following the manufacture's protocol. Cytotoxicity was determined using Cell Counting Kit-8 (CCK8, Dojindo Molecular Technologies, Inc.).

The compounds of the present invention were tested for their capacity to inhibit HBsAg and HBeAg as described herein. The Examples were tested in the above assay and found to have IC₅₀ below 10 μM. Results of PHH assay are given in Table 4.

TABLE 4
Activity data of compounds of this invention

| Example No. | HBsAg IC₅₀ (μM) | HBeAg IC₅₀ (μM) | CC₅₀ (μM) |
|---|---|---|---|
| Example 1 | 9.75 | 7.89 | 74.8 |
| Example 2 | 6.20 | 5.25 | 68.3 |
| Example 3 | 8.19 | 4.39 | >100.0 |
| Example 4 | 5.51 | 4.59 | >10.0 |
| Example 5 | 7.19 | 5.42 | >10.0 |
| Example 6 | 6.56 | 4.87 | >10.0 |
| Example 7 | 4.19 | 4.34 | >10.0 |
| Example 8 | 4.73 | 4.05 | >10.0 |
| Example 9 | 3.83 | 4.37 | >10.0 |
| Example 10 | 3.77 | 3.92 | >10.0 |
| Example 11 | 2.85 | 2.67 | >100.0 |
| Example 12 | 2.03 | 1.55 | >10.0 |
| Example 13 | 3.75 | 3.94 | >10.0 |
| Example 14 | 6.36 | 4.08 | >10.0 |
| Example 15 | 2.25 | 1.47 | >10.0 |
| Example 16 | 5.78 | 4.73 | >10.0 |
| Example 17 | 5.45 | 4.48 | >10.0 |
| Example 18 | 4.86 | 4.87 | >10.0 |
| Example 19 | 2.25 | 1.24 | >10.0 |
| Example 20 | 1.92 | 1.26 | >10.0 |
| Example 21 | 1.82 | 1.98 | 82.0 |
| Example 22 | 4.94 | 4.38 | >10.0 |
| Example 23 | 0.57 | 0.49 | 94.1 |
| Example 24 | 5.99 | 3.73 | >100.0 |
| Example 25 | 4.18 | 2.53 | >10.0 |
| Example 26 | 0.28 | 0.19 | 74.1 |
| Example 27 | 9.77 | 9.75 | >100.0 |
| Example 28 | 2.54 | 2.28 | >100.0 |
| Example 29 | 4.67 | 3.82 | >100.0 |
| Example 30 | 3.89 | 2.65 | >100.0 |
| Example 31 | 7.87 | 5.24 | >100.0 |
| Example 32 | 8.27 | 5.44 | >100.0 |
| Example 33 | 3.24 | 3.07 | 42.8 |
| Example 34 | 8.69 | 4.42 | >100.0 |
| Example 35 | 9.93 | 7.28 | >10.0 |
| Example 36 | 4.05 | 3.72 | >10.0 |
| Example 37 | 6.00 | 3.75 | >10.0 |
| Example 38 | 9.48 | 5.49 | >10.0 |
| Example 39 | 5.98 | 4.80 | >10.0 |
| Example 40 | 5.72 | 5.09 | >100.0 |
| Example 41 | 5.28 | 2.32 | >100.0 |
| Example 42 | 9.70 | 5.67 | >100.0 |
| Example 43 | 9.04 | 3.81 | >10.0 |
| Example 44 | 4.44 | 4.60 | >10.0 |
| Example 45 | 2.41 | 1.36 | >100.0 |
| Example 46 | 3.90 | 3.41 | >10.0 |
| Example 47 | 3.46 | 2.10 | >100.0 |
| Example 48 | 2.62 | 1.10 | >100.0 |
| Example 49 | 3.27 | 2.61 | 96.1 |
| Example 50 | 1.96 | 1.36 | >100.0 |
| Example 51 | 4.30 | 3.56 | >100.0 |
| Example 52 | 9.99 | 5.71 | >100.0 |
| Example 53 | 6.70 | 5.23 | >100.0 |
| Example 54 | 1.94 | 1.86 | >100.0 |
| Example 55 | 3.39 | 2.60 | 78.9 |
| Example 56 | 2.29 | 2.06 | >100.0 |
| Example 57 | 4.47 | 3.59 | >100.0 |
| Example 58 | 6.19 | 5.97 | >100.0 |
| Example 59 | 8.60 | 3.94 | >100.0 |
| Example 60 | 7.19 | 5.10 | >100.0 |
| Example 61 | 4.71 | 3.82 | >100.0 |
| Example 62 | 3.23 | 2.12 | >100.0 |
| Example 63 | 8.02 | 5.30 | >100.0 |
| Example 64 | 5.62 | 4.47 | 84.8 |
| Example 65 | 3.94 | 3.40 | >100.0 |
| Example 66 | 2.50 | 1.97 | 92.1 |
| Example 67 | 3.95 | 3.76 | >100.0 |
| Example 68 | 6.65 | 4.12 | >100.0 |
| Example 69 | 1.79 | 1.31 | >100.0 |
| Example 70 | 5.42 | 3.62 | >10.0 |
| Example 71 | 9.73 | 9.43 | >10.0 |
| Example 72 | 9.56 | 4.43 | >10.0 |
| Example 73 | 9.05 | 6.39 | >10.0 |
| Example 74 | 3.20 | 2.17 | >10.0 |
| Example 75 | 9.58 | 8.96 | >10.0 |
| Example 76 | 2.89 | 4.00 | >10.0 |
| Example 77 | 7.08 | 4.35 | >10.0 |
| Example 78 | 4.77 | 4.37 | >10.0 |
| Example 79 | 6.72 | 3.60 | >10.0 |
| Example 80 | 3.64 | 3.93 | >10.0 |
| Example 81 | 4.60 | 6.06 | >10.0 |
| Example 82a | 6.57 | 5.13 | >100.0 |
| Example 82b | 2.04 | 1.47 | >100.0 |
| Example 83 | 0.14 | 0.05 | 90.5 |
| Example 84a | 0.07 | 0.13 | 84.3 |
| Example 84b | 1.90 | 1.61 | >10.0 |
| Example 85 | 0.81 | 0.73 | >10.0 |
| Example 86 | 0.64 | 0.42 | >100.0 |
| Example 87-a | 0.20 | 0.22 | >10.0 |
| Example 87-b | 3.68 | 3.29 | >10.0 |
| Example 88 | 2.42 | 1.65 | >10.0 |
| Example 89 | 2.45 | 0.86 | >100.0 |
| Example 90 | 2.99 | 2.26 | >10.0 |
| Example 91 | 1.10 | 0.95 | >10.0 |
| Example 92 | 3.05 | 2.02 | >10.0 |
| Example 93 | 3.08 | 2.21 | >10.0 |
| Example 94c | 1.24 | 0.61 | >100.0 |
| Example 94d | 2.57 | 1.46 | >100.0 |
| Example 95 | 8.25 | 6.25 | >10.0 |
| Example 96 | 7.60 | 5.65 | >10.0 |
| Example 97 | 7.05 | 5.97 | >10.0 |
| Example 98 | 3.59 | 3.08 | >10.0 |
| Example 99 | 2.10 | 1.33 | >10.0 |
| Example 100 | 0.59 | 0.44 | >10.0 |
| Example 101 | 0.32 | 0.22 | >100.0 |
| Example 102 | 2.69 | 2.28 | >10.0 |
| Example 103 | 5.63 | 4.02 | >10.0 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the invention is not limited to the particular embodiments and aspects of the disclosure described above, as variations of the particular embodiments and aspects may be made and still fall within the scope of the appended claims. All documents cited to or relied upon herein are expressly incorporated by reference.

We claim:

1. A compound of the formula (I), 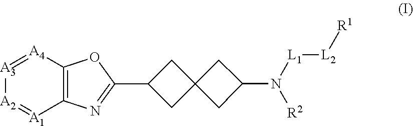
wherein
R¹ is C₁₋₆alkyl, C₃₋₇cycloalkyl, pyridyl, oxopyrrolidinyl, oxopiperidyl, aminosulfonyl, dioxothiolanyl, dioxothiazolidinyl, dioxothietanyl, azetidinyl, dioxo-2lambda6-thiaspiro[3.3]heptanyl, dioxothianyl, dioxothiazinanyl, tetrahydrofuranyl, pyrrolidinyl, 2-oxa-7-azaspiro[3.4]octanyl, piperidyl or C₃₋₇cycloalkylcarbonylaminosulfonyl; wherein C₃₋₇ cycloalkyl, pyridyl, oxopyrrolidinyl, oxopiperidyl, dioxothiazolidinyl, dioxothiolanyl, azetidinyl, dioxothianyl, pyrrolidinyl and piperidyl are unsubstituted or substituted by one or two or three substituents independently selected from OH, C₁₋₆alkyl, haloC₁₋₆alkyl, C₁₋₆alkoxy, oxetanylamino, (C₁₋₆alkyl)₂amino, piperidyl, morpholino, C₃₋₇cycloalkylamino, oxopyrrolidinyl, C₁₋₆alkylthiazolyl, thiazolyl, cyano, aminocarbonyl, aminosulfonyl, C₁₋₆ alkylaminosulfonylamino, C₁₋₆alkylsulfonyl, oxetanylsulfonyl, phenyl, halogen(C₁₋₆alkyl)phenyl, phenylC₁₋₆alkyl, C₃₋₇cycloalkylsulfonyl, C₁₋₆alkylcarbonyl, C₃₋₇cycloalkylcarbonyl, C₁₋₆alkylphenylsulfonyl, C₃₋₇cycloalkylsulfonimidoyl, C₁₋₆ alkylsulfonimidoyl, dioxothiazinanyl, N-hydroxycarbamimidoyl, carbamimidoyl, C₁₋₆ alkylamino, azetidinylsulfonyl, C₁₋₆alkylcarbonylaminosulfonyl, C₃₋₇cycloalkylC₁₋₆alkylsulfonyl, C₁₋₆alkylaminocarbonyl and (C₁₋₆alkyl)₂aminocarbonyl;
R² is H or C₁₋₆alkyl;
L₁ is —C(O)— or —S(O)₂—;
L₂ is a bond, —(CH₂)ₘ—, —NH—, N(CH₃)— or —NH—CH₂—; wherein m is 1, 2 or 3;
A₁ is N or CR³; wherein R³ is H, halogen, C₁₋₆alkyl or haloC₁₋₆alkyl;
A₂ is N or CR⁴; wherein R⁴ is H, halogen, C₁₋₆alkyl or haloC₁₋₆alkyl;
A₃ is N or CR⁵; wherein R⁵ is H, halogen, C₁₋₆alkyl or haloC₁₋₆alkyl;
A₄ is N or CR⁶; wherein R⁶ is H, halogen, C₁₋₆alkyl or haloC₁₋₆alkyl;
wherein with the proviso that A₁, A₂, A₃ and A₄ are not CH simultaneously;
or a pharmaceutically acceptable salt thereof.

2. A compound according to claim 1, or a pharmaceutically acceptable salt thereof, wherein A₁ is N or CH.

3. A compound according to claim 1, or a pharmaceutically acceptable salt thereof, wherein A₁ is CH.

4. A compound according to claim 1, or a pharmaceutically acceptable salt thereof, wherein A₂ is CR⁴; wherein R⁴ is H, halogen or haloC₁₋₆alkyl.

5. A compound according to claim 1, or a pharmaceutically acceptable salt thereof, wherein A₂ is CR⁴; wherein R⁴ is halogen.

6. A compound according to claim 5, or a pharmaceutically acceptable salt thereof, wherein A₂ is CCl.

7. A compound according to claim 1, or a pharmaceutically acceptable salt thereof, wherein A₃ is CH.

8. A compound according to claim 1, or a pharmaceutically acceptable salt thereof, wherein A₄ is N or CH.

9. A compound according to claim 1, or a pharmaceutically acceptable salt thereof, wherein R² is H or methyl.

10. A compound according to claim 9, or a pharmaceutically acceptable salt thereof, wherein R² is H.

11. A compound according to claim 1, or a pharmaceutically acceptable salt thereof, wherein L₁ is —C(O)—.

12. A compound according to claim 1, or a pharmaceutically acceptable salt thereof, wherein L₂ is a bond.

13. A compound according to claim 1, or a pharmaceutically acceptable salt thereof, wherein R¹ is pyridyl; wherein pyridyl is substituted by one substituent selected from aminocarbonyl, C₁₋₆alkylsulfonyl, C₃₋₇cycloalkylsulfonyl, C₃₋₇cycloalkylsulfonimidoyl and C₃₋₇cycloalkylC₁₋₆alkylsulfonyl.

14. A compound according to claim 1, or a pharmaceutically acceptable salt thereof, wherein R¹ is pyridyl; wherein pyridyl is substituted by one substituent selected from aminocarbonyl, methylsulfonyl, ethylsulfonyl, cyclopropylsulfonyl, cyclopropylsulfonimidoyl and cyclopropylmethylsulfonyl.

15. A compound according to claim 1, wherein
R¹ is C₁₋₆alkyl, C₃₋₇cycloalkyl, pyridyl, oxopyrrolidinyl, oxopiperidyl, aminosulfonyl, dioxothiolanyl, dioxothiazolidinyl, dioxothietanyl, azetidinyl, dioxo-2lambda6-thiaspiro[3.3]heptanyl, dioxothianyl, dioxothiazinanyl, tetrahydrofuranyl, pyrrolidinyl, 2-oxa-7-azaspiro[3.4]octanyl, piperidyl or C₃₋₇cycloalkylcarbonylaminosulfonyl; wherein C₃₋₇cycloalkyl, pyridyl, oxopyrrolidinyl, oxopiperidyl, dioxothiazolidinyl, dioxothiolanyl, azetidinyl, dioxothianyl, pyrrolidinyl and piperidyl are unsubstituted or substituted by one or two or three substituents independently selected from OH, C₁₋₆alkyl, haloC₁₋₆alkyl, C₁₋₆alkoxy, oxetanylamino, (C₁₋₆alkyl)₂amino, piperidyl, morpholino, C₃₋₇cycloalkylamino, oxopyrrolidinyl, C₁₋₆alkylthiazolyl, thiazolyl, cyano, aminocarbonyl, aminosulfonyl, C₁₋₆alkylaminosulfonylamino, C₁₋₆alkylsulfonyl, oxetanylsulfonyl, phenyl, halogen(C₁₋₆ alkyl)phenyl, phenylC₁₋₆alkyl, C₃₋₇cycloalkylsulfonyl, C₁₋₆alkylcarbonyl, C₃₋₇cycloalkylcarbonyl, C₁₋₆alkylphenylsulfonyl, C₃₋₇cycloalkylsulfonimidoyl, C₁₋₆ alkylsulfonimidoyl, dioxothiazinanyl, N-hydroxycarbamimidoyl, carbamimidoyl, C₁₋₆alkylamino, azetidinylsulfonyl, C₁₋₆alkylcarbonylaminosulfonyl, C₃₋₇cycloalkylC₁₋₆alkylsulfonyl, C₁₋₆alkylaminocarbonyl and (C₁₋₆alkyl)₂aminocarbonyl;
R² is H or C₁₋₆alkyl;
L₁ is —C(O)— or —S(O)₂—;
L₂ is a bond, —(CH₂)ₘ—, —NH—, N(CH₃)— or —NH—CH₂—; wherein m is 1, 2 or 3;
A₁ is N or CH;
A₂ is CR⁴; wherein R⁴ is H, halogen or haloC₁₋₆alkyl;
A₃ is CH;
A₄ is N or CH;
wherein with the proviso that A₁, A₂, A₃ and A₄ are not CH simultaneously;
or a pharmaceutically acceptable salt thereof.

16. A compound according to claim 1, wherein
R¹ pyridyl; wherein pyridyl is substituted by one substituent selected from aminocarbonyl, C₁₋₆alkylsulfonyl, C₃₋₇cycloalkylsulfonyl, C₃₋₇cycloalkylsulfonimidoyl and C₃₋₇cycloalkylC₁₋₆alkylsulfonyl;
R² is H;
L₁ is —C(O)—;
L₂ is a bond;
A₁ is CH;
A₂ is CR⁴; wherein R⁴ is halogen;
A₃ is CH;
A₄ is N or CH;
wherein with the proviso that A₁, A₂, A₃ and A₄ are not CH simultaneously;
or a pharmaceutically acceptable salt thereof.

17. A compound according to claim 1, wherein
R¹ is pyridyl; wherein pyridyl is substituted by one substituent selected from aminocarbonyl, methylsulfonyl, ethylsulfonyl, cyclopropylsulfonyl, cyclopropylsulfonimidoyl and cyclopropylmethylsulfonyl;
R² is H;
L₁ is —C(O)—;
L₂ is a bond;
A₁ is CH;
A₂ is CCl;
A₃ is CH;
A₄ is N or CH;
wherein with the proviso that A₁, A₂, A₃ and A₄ are not CH simultaneously;
or a pharmaceutically acceptable salt thereof.

18. A compound according to claim 1, selected from the group consisting of:
N-[6-(5-chloro-1,3-benzoxazol-2-yl)spiro[3.3]heptan-2-yl]pyridine-2-carboxamide;
N-[6-(5-chloro-1,3-benzoxazol-2-yl)spiro[3.3]heptan-2-yl]pyridine-3-carboxamide;
N-[6-(5-chloro-1,3-benzoxazol-2-yl)spiro[3.3]heptan-2-yl]pyridine-4-carboxamide;
N-[6-(5-chloro-1,3-benzoxazol-2-yl)spiro[3.3]heptan-2-yl]-2-methyl-pyridine-4-carboxamide;
N-[6-(5-chloro-1,3-benzoxazol-2-yl)spiro[3.3]heptan-2-yl]-2-(oxetan-3-ylamino)pyridine-4-carboxamide;
N-[6-(5-chloro-1,3-benzoxazol-2-yl)spiro[3.3]heptan-2-yl]-2-(diethylamino)pyridine-4-carboxamide;
N-[6-(5-chloro-1,3-benzoxazol-2-yl)spiro[3.3]heptan-2-yl]-2-[ethyl(methyl)amino]pyridine-4-carboxamide;
N-[6-(5-chloro-1,3-benzoxazol-2-yl)spiro[3.3]heptan-2-yl]-2-(1-piperidyl)pyridine-4-carboxamide;
N-[6-(5-chloro-1,3-benzoxazol-2-yl)spiro[3.3]heptan-2-yl]-2-morpholino-pyridine-4-carboxamide;
N-[6-(5-chloro-1,3-benzoxazol-2-yl)spiro[3.3]heptan-2-yl]-2-(cyclopropylamino)pyridine-4-carboxamide;
N-[6-(5-chloro-1,3-benzoxazol-2-yl)spiro[3.3]heptan-2-yl]-2-(dimethylamino)pyridine-4-carboxamide;
N-[6-(5-chloro-1,3-benzoxazol-2-yl)spiro[3.3]heptan-2-yl]-2-[isopropyl(methyl)amino]pyridine-4-carboxamide;
N-[6-(5-chloro-1,3-benzoxazol-2-yl)spiro[3.3]heptan-2-yl]-2-(2-oxopyrrolidin-1-yl)pyridine-4-carboxamide;
N-[6-(5-chloro-1,3-benzoxazol-2-yl)spiro[3.3]heptan-2-yl]-2-(4-methylthiazol-2-yl)pyridine-4-carboxamide;
N-[6-(5-chloro-1,3-benzoxazol-2-yl)spiro[3.3]heptan-2-yl]-2-(5-methylthiazol-2-yl)pyridine-4-carboxamide;
N-[6-(5-chloro-1,3-benzoxazol-2-yl)spiro[3.3]heptan-2-yl]-2-thiazol-2-yl-pyridine-4-carboxamide;
N-[6-(5-chloro-1,3-benzoxazol-2-yl)spiro[3.3]heptan-2-yl]-2-methoxy-pyridine-4-carboxamide;
N-[6-(5-chloro-1,3-benzoxazol-2-yl)spiro[3.3]heptan-2-yl]-2-cyano-pyridine-4-carboxamide;
N-[6-(5-chloro-1,3-benzoxazol-2-yl)spiro[3.3]heptan-2-yl]-2-sulfamoyl-pyridine-4-carboxamide;
N-[6-(5-chloro-1,3-benzoxazol-2-yl)spiro[3.3]heptan-2-yl]-2-isopropoxy-pyridine-4-carboxamide;
N-[6-(5-chloro-1,3-benzoxazol-2-yl)spiro[3.3]heptan-2-yl]-2-(trifluoromethyl)pyridine-4-carboxamide;
N-[6-(5-chloro-1,3-benzoxazol-2-yl)spiro[3.3]heptan-2-yl]-2-(methylsulfamoylamino)pyridine-4-carboxamide;
N-[6-(5-chloro-1,3-benzoxazol-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-5-methylsulfonyl-pyridine-3-carboxamide;
N-[6-(5-chloro-1,3-benzoxazol-2-yl)spiro[3.3]heptan-2-yl]-2-(oxetan-3-ylsulfonyl)pyridine-4-carboxamide;
N-[6-(5-chloro-1,3-benzoxazol-2-yl)spiro[3.3]heptan-2-yl]-2-ethylsulfonyl-pyridine-4-carboxamide;
N-[6-(5-chloro-1,3-benzoxazol-2-yl)spiro[3.3]heptan-2-yl]-2,2-dimethyl-propanamide;
N-[6-(5-chloro-1,3-benzoxazol-2-yl)spiro[3.3]heptan-2-yl]-3-methyl-butanamide;
N-[6-(5-chloro-1,3-benzoxazol-2-yl)spiro[3.3]heptan-2-yl]-3-(trifluoromethyl)cyclobutanecarboxamide;
N-[6-(5-chloro-1,3-benzoxazol-2-yl)spiro[3.3]heptan-2-yl]-1-methyl-5-oxo-pyrrolidine-3-carboxamide;
1-tert-butyl-N-[6-(5-chloro-1,3-benzoxazol-2-yl)spiro[3.3]heptan-2-yl]-5-oxo-pyrrolidine-3-carboxamide;
N-[6-(5-chloro-1,3-benzoxazol-2-yl)spiro[3.3]heptan-2-yl]-5-oxo-1-phenyl-pyrrolidine-3-carboxamide;
N-[6-(5-chloro-1,3-benzoxazol-2-yl)spiro[3.3]heptan-2-yl]-1-(3-chloro-2-methyl-phenyl)-5-oxo-pyrrolidine-3-carboxamide;
N-[6-(5-chloro-1,3-benzoxazol-2-yl)spiro[3.3]heptan-2-yl]-1-methyl-2-oxo-piperidine-4-carboxamide;
N-[6-(5-chloro-1,3-benzoxazol-2-yl)spiro[3.3]heptan-2-yl]-2-(1-methyl-5-oxo-pyrrolidin-3-yl)acetamide;
N-[6-(5-chloro-1,3-benzoxazol-2-yl)spiro[3.3]heptan-2-yl]-2-(1-methyl-5-oxo-pyrrolidin-2-yl)acetamide;
N-[6-(5-chloro-1,3-benzoxazol-2-yl)spiro[3.3]heptan-2-yl]-2-(5-oxopyrrolidin-2-yl)acetamide;
N-[6-(5-chloro-1,3-benzoxazol-2-yl)spiro[3.3]heptan-2-yl]-3-sulfamoyl-propanamide;
N-[6-(5-chloro-1,3-benzoxazol-2-yl)spiro[3.3]heptan-2-yl]-4-sulfamoyl-butanamide;
N-[6-(5-chloro-1,3-benzoxazol-2-yl)spiro[3.3]heptan-2-yl]-1,1-dioxo-thiolane-3-carboxamide;
N-(6-oxazolo[4,5-b]pyridin-2-ylspiro[3.3]heptan-2-yl)-1,1-dioxo-thiolane-3-carboxamide;
N-[6-(5-chloro-1,3-benzoxazol-2-yl)spiro[3.3]heptan-2-yl]-1,1-dioxo-thiolane-2-carboxamide;
N-[6-(5-chloro-1,3-benzoxazol-2-yl)spiro[3.3]heptan-2-yl]-1,1-dioxo-1,2-thiazolidine-4-carboxamide;
2-benzyl-N-[6-(5-chloro-1,3-benzoxazol-2-yl)spiro[3.3]heptan-2-yl]-1,1-dioxo-1,2-thiazolidine-4-carboxamide;
N-[6-(5-chloro-1,3-benzoxazol-2-yl)spiro[3.3]heptan-2-yl]-1,1-dioxo-1,2-thiazolidine-3-carboxamide;
N-[6-(5-chloro-1,3-benzoxazol-2-yl)spiro[3.3]heptan-2-yl]-2-methyl-1,1-dioxo-1,2-thiazolidine-3-carboxamide;
N-[6-(5-chloro-1,3-benzoxazol-2-yl)spiro[3.3]heptan-2-yl]-2-(1,1-dioxothiolan-2-yl)acetamide;
N-(6-(5-chlorobenzo[d]oxazol-2-yl)spiro[3.3]heptan-2-yl)-3-methyltetrahydrothiophene-3-carboxamide 1,1-dioxide;
N-[6-(5-chloro-1,3-benzoxazol-2-yl)spiro[3.3]heptan-2-yl]-1,1-dioxo-thietane-3-carboxamide;
N-[6-(5-chloro-1,3-benzoxazol-2-yl)spiro[3.3]heptan-2-yl]-2-(1,1-dioxothietan-3-yl)acetamide;
N-[6-(5-chloro-1,3-benzoxazol-2-yl)spiro[3.3]heptan-2-yl]oxetane-3-carboxamide;
N-[6-(5-chloro-1,3-benzoxazol-2-yl)spiro[3.3]heptan-2-yl]-2,2-dioxo-2-thiaspiro[3.3]heptane-6-carboxamide;
N-[6-(5-chloro-1,3-benzoxazol-2-yl)spiro[3.3]heptan-2-yl]-1,1-dioxo-thiane-4-carboxamide;
N-(6-(5-chlorobenzo[d]oxazol-2-yl)spiro[3.3]heptan-2-yl)-4-methyltetrahydro-2H-thiopyran-4-carboxamide 1,1-dioxide;
N-[6-(5-chloro-1,3-benzoxazol-2-yl)spiro[3.3]heptan-2-yl]-2-(1,1-dioxothian-3-yl)acetamide;
N-[6-(5-chloro-1,3-benzoxazol-2-yl)spiro[3.3]heptan-2-yl]-2-(1,1-dioxothiazinan-2-yl)acetamide;
1,1-dioxo-N-[6-[5-(trifluoromethyl)-1,3-benzoxazol-2-yl]spiro[3.3]heptan-2-yl]thiane-4-carboxamide;
N-[6-(5-chloro-1,3-benzoxazol-2-yl)spiro[3.3]heptan-2-yl]propane-2-sulfonamide;
1-[6-(5-chloro-1,3-benzoxazol-2-yl)spiro[3.3]heptan-2-yl]-3-ethyl-urea;
1-[6-(5-chloro-1,3-benzoxazol-2-yl)spiro[3.3]heptan-2-yl]-3-propyl-urea;
1-[6-(5-chloro-1,3-benzoxazol-2-yl)spiro[3.3]heptan-2-yl]-3-isopropyl-urea;
3-[6-(5-chloro-1,3-benzoxazol-2-yl)spiro[3.3]heptan-2-yl]-1-ethyl-1-methyl-urea;
1-[6-(5-chloro-1,3-benzoxazol-2-yl)spiro[3.3]heptan-2-yl]-3-(tetrahydrofuran-3-ylmethyl)urea;
1-[6-(5-chloro-1,3-benzoxazol-2-yl)spiro[3.3]heptan-2-yl]-3-[(3-hydroxycyclobutyl)methyl]urea;
1-[6-(5-chloro-1,3-benzoxazol-2-yl)spiro[3.3]heptan-2-yl]-3-tetrahydrofuran-3-yl-urea;
N-[6-(5-chloro-1,3-benzoxazol-2-yl)spiro[3.3]heptan-2-yl]pyrrolidine-1-carboxamide;
N-[6-(5-chloro-1,3-benzoxazol-2-yl)spiro[3.3]heptan-2-yl]-2-oxa-7-azaspiro[3.4]octane-7-carboxamide;
N-[6-(5-chloro-1,3-benzoxazol-2-yl)spiro[3.3]heptan-2-yl]-1,1-dioxo-1,4-thiazinane-4-carboxamide;
N-[6-(5-chloro-1,3-benzoxazol-2-yl)spiro[3.3]heptan-2-yl]-1-methylsulfonyl-pyrrolidine-3-carboxamide;
N-[6-(5-chloro-1,3-benzoxazol-2-yl)spiro[3.3]heptan-2-yl]-1-ethylsulfonyl-pyrrolidine-3-carboxamide;
N-[6-(5-chloro-1,3-benzoxazol-2-yl)spiro[3.3]heptan-2-yl]-1-propylsulfonyl-pyrrolidine-3-carboxamide;
N-[6-(5-chloro-1,3-benzoxazol-2-yl)spiro[3.3]heptan-2-yl]-1-cyclopropylsulfonyl-pyrrolidine-3-carboxamide;
1-acetyl-N-[6-(5-chloro-1,3-benzoxazol-2-yl)spiro[3.3]heptan-2-yl]pyrrolidine-3-carboxamide;
N-[6-(5-chloro-1,3-benzoxazol-2-yl)spiro[3.3]heptan-2-yl]-1-(cyclopropanecarbonyl)pyrrolidine-3-carboxamide;
N-[6-(5-chloro-1,3-benzoxazol-2-yl)spiro[3.3]heptan-2-yl]-1-(cyclobutanecarbonyl)pyrrolidine-3-carboxamide;
N-[6-(5-chloro-1,3-benzoxazol-2-yl)spiro[3.3]heptan-2-yl]-2-[1-(p-tolylsulfonyl)pyrrolidin-3-yl]acetamide;
N-[6-(5-chloro-1,3-benzoxazol-2-yl)spiro[3.3]heptan-2-yl]-2-(1-methylsulfonylazetidin-3-yl)acetamide;
N-[6-(5-chloro-1,3-benzoxazol-2-yl)spiro[3.3]heptan-2-yl]-2-[1-(p-tolylsulfonyl)azetidin-3-yl]acetamide;
N-[6-(5-chloro-1,3-benzoxazol-2-yl)spiro[3.3]heptan-2-yl]-2-(1-methylsulfonyl-4-piperidyl)acetamide;
N-[6-(5-chloro-1,3-benzoxazol-2-yl)spiro[3.3]heptan-2-yl]-2-[1-(p-tolylsulfonyl)-4-piperidyl]acetamide;
N-[4-[[6-(5-chloro-1,3-benzoxazol-2-yl)spiro[3.3]heptan-2-yl]amino]-4-oxo-butyl]sulfonylcyclobutanecarboxamide;
(Rₐ)—N-[6-(5-chloro-1,3-benzoxazol-2-yl)spiro[3.3]heptan-2-yl]-1,1-dioxo-thiane-4-carboxamide;
(Sₐ)—N-[6-(5-chloro-1,3-benzoxazol-2-yl)spiro[3.3]heptan-2-yl]-1,1-dioxo-thiane-4-carboxamide;
N-[6-(5-chloro-1,3-benzoxazol-2-yl)spiro[3.3]heptan-2-yl]-2-cyclopropylsulfonyl-pyridine-4-carboxamide;
(Rₐ)—N-[6-(5-chloro-1,3-benzoxazol-2-yl)spiro[3.3]heptan-2-yl]-2-cyclopropylsulfonyl-pyridine-4-carboxamide;
(Sₐ)—N-[6-(5-chloro-1,3-benzoxazol-2-yl)spiro[3.3]heptan-2-yl]-2-cyclopropylsulfonyl-pyridine-4-carboxamide;
N-[6-(6-chlorooxazolo[5,4-b]pyridin-2-yl)spiro[3.3]heptan-2-yl]-2-cyclopropylsulfonyl-pyridine-4-carboxamide;
N-[6-(5-chloro-1,3-benzoxazol-2-yl)spiro[3.3]heptan-2-yl]-2-(cyclopropylsulfonimidoyl)pyridine-4-carboxamide;
(Rₐ)—N-[6-(5-chloro-1,3-benzoxazol-2-yl)spiro[3.3]heptan-2-yl]-2-methylsulfonyl-pyridine-4-carboxamide;
(Sₐ)—N-[6-(5-chloro-1,3-benzoxazol-2-yl)spiro[3.3]heptan-2-yl]-2-methylsulfonyl-pyridine-4-carboxamide;
N-[6-(6-chlorooxazolo[5,4-b]pyridin-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-2-(methylsulfonimidoyl)pyridine-4-carboxamide;
5-chloro-N-[6-(5-chloro-1,3-benzoxazol-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-2-(1,1-dioxo-1,4-thiazinan-4-yl)pyridine-4-carboxamide;
N-[6-(5-chloro-1,3-benzoxazol-2-yl)spiro[3.3]heptan-2-yl]-N-methyl-2-methylsulfonyl-pyridine-4-carboxamide;
N-[6-(5-chloro-1,3-benzoxazol-2-yl)spiro[3.3]heptan-2-yl]-2-ethylsulfonyl-N-methyl-pyridine-4-carboxamide;
(Rₐ)-(3R)—N-[6-(5-chloro-1,3-benzoxazol-2-yl)spiro[3.3]heptan-2-yl]-1-methyl-5-oxo-pyrrolidine-3-carboxamide;
(Rₐ)-(3S)—N-[6-(5-chloro-1,3-benzoxazol-2-yl)spiro[3.3]heptan-2-yl]-1-methyl-5-oxo-pyrrolidine-3-carboxamide;
(Sₐ)-(3R)—N-[6-(5-chloro-1,3-benzoxazol-2-yl)spiro[3.3]heptan-2-yl]-1-methyl-5-oxo-pyrrolidine-3-carboxamide;
(Sₐ)-(3S)—N-[6-(5-chloro-1,3-benzoxazol-2-yl)spiro[3.3]heptan-2-yl]-1-methyl-5-oxo-pyrrolidine-3-carboxamide;
N-[6-(5-chloro-1,3-benzoxazol-2-yl)spiro[3.3]heptan-2-yl]-2-(N-hydroxycarbamimidoyl)pyridine-4-carboxamide;
2-carbamimidoyl-N-[6-(5-chloro-1,3-benzoxazol-2-yl)spiro[3.3]heptan-2-yl]pyridine-4-carboxamide;
N-[6-(5-chloro-1,3-benzoxazol-2-yl)spiro[3.3]heptan-2-yl]-2-(methylamino)pyridine-4-carboxamide;
2-(azetidin-3-ylsulfonyl)-N-[6-(5-chloro-1,3-benzoxazol-2-yl)spiro[3.3]heptan-2-yl]pyridine-4-carboxamide;
2-(acetylsulfamoyl)-N-[6-(5-chloro-1,3-benzoxazol-2-yl)spiro[3.3]heptan-2-yl]pyridine-4-carboxamide;
N-[6-(5-chloro-1,3-benzoxazol-2-yl)spiro[3.3]heptan-2-yl]-2-(cyclopropylmethylsulfonyl)pyridine-4-carboxamide;
N4-[6-(5-chloro-1,3-benzoxazol-2-yl)spiro[3.3]heptan-2-yl]pyridine-2,4-dicarboxamide;
N4-[6-(5-chloro-1,3-benzoxazol-2-yl)spiro[3.3]heptan-2-yl]-N2-methyl-pyridine-2,4-dicarboxamide; and
N4-[6-(5-chloro-1,3-benzoxazol-2-yl)spiro[3.3]heptan-2-yl]-N2,N2-dimethyl-pyridine-2,4-dicarboxamide;
or a pharmaceutically acceptable salt thereof.

19. A compound according to claim 1, selected from the group consisting of:
N-[6-(5-chloro-1,3-benzoxazol-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-2-ethylsulfonyl-pyridine-4-carboxamide;
N-[6-(5-chloro-1,3-benzoxazol-2-yl)spiro[3.3]heptan-2-yl]-2-cyclopropylsulfonyl-pyridine-4-carboxamide
(Rₐ)—N-[6-(5-chloro-1,3-benzoxazol-2-yl)spiro[3.3]heptan-2-yl]-2-cyclopropylsulfonyl-pyridine-4-carboxamide;
(Sₐ)—N-[6-(5-chloro-1,3-benzoxazol-2-yl)spiro[3.3]heptan-2-yl]-2-cyclopropylsulfonyl-pyridine-4-carboxamide;
N-[6-(6-chlorooxazolo[5,4-b]pyridin-2-yl)spiro[3.3]heptan-2-yl]-2-cyclopropylsulfonyl-pyridine-4-carboxamide;
N-[6-(5-chloro-1,3-benzoxazol-2-yl)spiro[3.3]heptan-2-yl]-2-(cyclopropylsulfonimidoyl)pyridine-4-carboxamide;
(Rₐ)—N-[6-(5-chloro-1,3-benzoxazol-2-yl)spiro[3.3]heptan-2-yl]-2-methylsulfonyl-pyridine-4-carboxamide;
(Sₐ)—N-[6-(5-chloro-1,3-benzoxazol-2-yl)spiro[3.3]heptan-2-yl]-2-methylsulfonyl-pyridine-4-carboxamide;
N-[6-(5-chloro-1,3-benzoxazol-2-yl)spiro[3.3]heptan-2-yl]-2-(cyclopropylmethylsulfonyl)pyridine-4-carboxamide; and,
N4-[6-(5-chloro-1,3-benzoxazol-2-yl)spiro[3.3]heptan-2-yl]pyridine-2,4-dicarboxamide;
or a pharmaceutically acceptable salt thereof.

20. A process for the preparation of a compound according to claim 1 comprising the following step,
(a) Reaction of a compound of formula (IV), 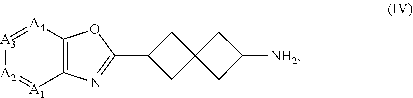
with a compound of formula (V), 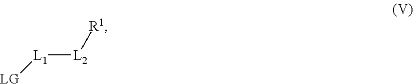
in the presence of a base;
(b) Reaction of a compound of formula (VIII), 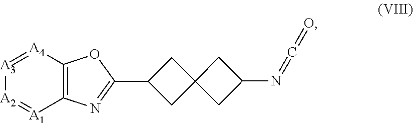
with a compound of formula (IX), 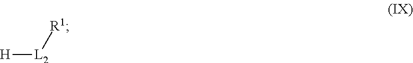
(c) Reaction of a compound of formula (XI), 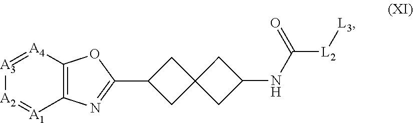
with a compound of formula (XII), Cl—R⁷ (XII); in the presence of a base;
(d) Oxidation of a compound of formula (XIII), 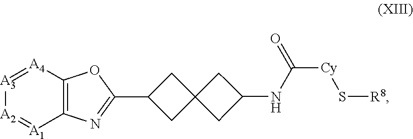
in the presence of an oxidate and (NH₄)₂CO;
(e) Reaction of a compound of formula (XIV), 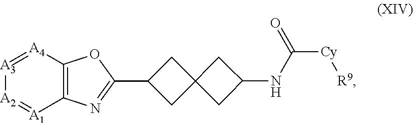
with a compound of formula (XV), 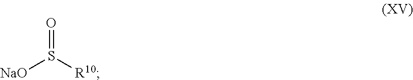
in the presence of an catalysts and a base;
(f) Reaction of a compound of formula (XVI), 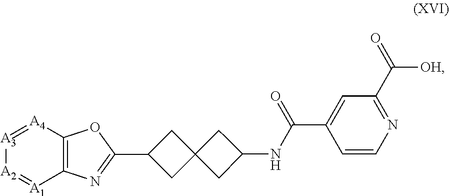
with a compound of formula (IX), H—R¹¹ (IX); in the presence of a coupling reagent and a base;
wherein A₁ to A₄, L₁, L₂ and R¹ are defined as claims 1; L₃ is azetidinyl, pyrrolidinyl, piperidyl or —S(O)₂NH₂; L₄ is S(O)₂ or S(O)(NH); R⁷ is C₃₋₇cycloalkylsulfonyl, C₁₋₆alkylcarbonyl, C₃₋₇cycloalkylcarbonyl or C₁₋₆alkylphenylsulfonyl; L₄ is S(O)₂ or S(O)(NH); R⁸ is C₁₋₆alkyl, C₃₋₇cycloalkyl; R⁹ is halogen; R¹⁰ is C₁₋₆alkyl, C₃₋₇cycloalkyl; R¹¹ is —NH, —NHCH₃ or —N(CH₃)₂; Cy is pyridyl or chloropyridyl.

21. A compound, or a pharmaceutically acceptable salt thereof, when manufactured according to a process of claim 20.

22. A compound according to claim 1, or a pharmaceutically acceptable salt thereof, for use as therapeutically active substance.

23. A pharmaceutical composition comprising a compound according to claim 1, or a pharmaceutically acceptable salt thereof, and a pharmaceutically acceptable excipient.

24. The use of a compound according to claim 1, or a pharmaceutically acceptable salt thereof, for the treatment of HBV infection.

25. The use of a compound according to claim 1, or a pharmaceutically acceptable salt thereof, for the preparation of a medicament for the treatment of HBV infection.

26. The use of a compound according to claim 1, or a pharmaceutically acceptable salt thereof, for the inhibition of HBeAg.

27. The use of a compound according to claim 1, or a pharmaceutically acceptable salt thereof, for the inhibition of HBsAg.

28. The use of a compound according to claim 1, or a pharmaceutically acceptable salt thereof, for the inhibition of HBV DNA.

29. A compound according to claim 1, or a pharmaceutically acceptable salt thereof, for use in the treatment of HBV infection.

30. A method for the treatment of HBV infection, which method comprises administering an effective amount of a compound as defined in claim 1, or a pharmaceutically acceptable salt thereof.
